(19) United States
(12) Patent Application Publication
(10) Pub. No.: US 2024/0425453 A1
(43) Pub. Date: Dec. 26, 2024

(54) ISOINDOLINES AS PMS2 INHIBITORS

(71) Applicant: NeoPhore Limited, Altrincham Cheshire (GB)

(72) Inventors: Julian Blagg, Greater Manchester (GB); Martin Drysdale, Greater Manchester (GB); David Clark, Essex (GB); Paul Winship, Essex (GB)

(21) Appl. No.: 18/712,311

(22) PCT Filed: Nov. 28, 2022
(86) PCT No.: PCT/GB22/53006

§ 371 Date: May 22, 2024

(30) Foreign Application Priority Data
Nov. 29, 2021 (GB) 2117224.2

(51) Int. Cl. C07D 209/44; A61K 31/4035; A61K 31/47; A61K 31/496; A61K 31/5377; A61K 45/06; C07D 217/20; C07D 403/12

(52) U.S. Cl. CPC C07D 209/44; A61K 31/4035; A61K 31/47; A61K 31/496; A61K 31/5377; A61K 45/06; C07D 217/20; C07D 403/12

(57) ABSTRACT

The present invention relates to compounds of Formula (II) that target the PMS2 proteins that are components of the DNA Mismatch Repair (MMR) process:

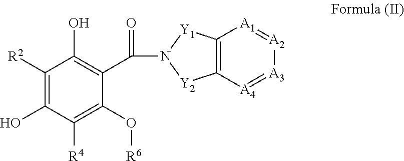

wherein R², R⁴, R⁶, Y₁, Y₂, A₁, A₂, A₃, and A₄ are each as defined herein. The present invention also relates to processes for the preparation of these compounds, to pharmaceutical compositions comprising them, and to their use in the treatment of proliferative disorders, such as cancer, as well as other diseases or conditions in which PMS2 activity is implicated.

### INTRODUCTION

The present invention relates to certain compounds that function as inhibitors of PMS2 protein activity. In particular, the compounds of the present invention may be used as covalent binders to inhibit PMS2. Thus, the compounds of the present invention may be used to treat disease or conditions mediated, at least in part, by inappropriate PMS2 activity, for example, cancer. The invention furthermore relates to the use of the compounds and pharmaceutical compositions comprising them.

### BACKGROUND OF THE INVENTION

Cancer is caused by altered cellular proliferation. Precisely what causes a cell to become malignant and proliferate in an uncontrolled and unregulated manner has been the focus of intense research over recent decades. This research has led to the identification of molecular targets associated with key pathways that enable such malignancies.

Mismatch repair (MMR) is a highly conserved DNA repair pathway that plays a major role during DNA replication, repair and recombination, as well as during meiosis in eukaryotes and immunoglobulin maturation/diversification in mammals. MMR promotes genome stability in all organisms by correcting DNA base mismatches and insertion/deletion (indel) loops that can occasionally arise during normal DNA replication process. Base pair mismatches occur when incorrect nucleotides are inserted into the newly synthesized DNA strand and escape the proofreading function of DNA polymerases. Indel loops commonly arise in the context of microsatellites—highly polymorphic short repetitive DNA sequences distributed throughout both prokaryotic and eukaryotic genomes. Typically, at microsatellites, the template and primer strands are prone to slippage (dissociation and reannealing) during replication, which can generate loop structures and a discordant number of repeat units between the template and newly synthesized strand.

DNA mismatch repair is a bidirectional excision and re-synthesis system that initiates at a defined strand scission 3′- or 5′- to a mismatch; the excision tract extends just past the mismatch. MMR can be divided into four steps: 1) mismatch recognition by MSH proteins; 2) recruitment of MLH/PMS proteins that connect the mismatch recognition signal to where the distant DNA strand scission begins; 3) excision of the errant DNA strand, and 4) re-synthesis of the excision gap using the remaining DNA strand as a template [1]. MMR is a highly conserved biological pathway. In humans, mismatch recognition by hMutSa (MSH2-MSH6) or hMutSs (MSH2-MSH3) initiates the MMR pathway. Binding of hMutSa or hMutSB to the mismatch site results in the recruitment of MutLα (MLH1-PMS2) to form a ternary complex whose protein-protein, protein-DNA interactions and endonuclease activity are modulated by ATP/ADP cofactors. Proliferating cell nuclear antigen (PCNA) may play a role in the recruitment of MMR proteins to the vicinity of the replication fork [1]. PCNA may also activate a latent endonuclease activity in eukaryotic MutLα proteins. After DNA incision, exonuclease 1 (EXO1) is recruited which excises the newly synthesized DNA strand and the DNA excision gap is re-synthesized by DNA polymerase 6 (Pol S). When DNA re-synthesis is complete, the remaining nick is ligated by DNA ligase to restore the integrity of the duplex [2]. Consistent with this function, MMR is an important tumor suppressor pathway that is lost in up to 40% of sporadic cancers. Moreover, individuals with germline mutations in MMR genes develop cancer predisposition conditions.

Lynch Syndrome (LS, formerly designated as hereditary non-polyposis colorectal cancer) is the most common cause of hereditary colorectal cancer (CRC), accounting for 2-5% of all cases. LS is also characterized by an increased risk of malignancies at certain extracolonic sites such as the endometrium, ovary, stomach and small bowel, among others [3]. LS has an autosomal dominant inheritance pattern and is caused by germline mutations in MMR genes MLH1, MSH2, MSH6 or PMS2. Gene expression from the one wild-type allele is sufficient for adequate MMR activity until a second hit inactivates the wild-type allele leading to MMR deficiency.

Constitutional mismatch repair deficiency (CMMRD) syndrome is a distinct childhood cancer predisposition syndrome that results from biallelic germline mutations in one of four MMR genes, MLH1, MSH2, MSH6 or PMS2. Patients may have either homozygous biallelic alterations or heterozygous alterations of MMR genes.

MMR-deficient cancers are commonly and typically characterized by the accumulation of DNA mutations at higher rates than normal cells and other tumours; for example, CMMRD tumours commonly have an ultra-hypermutated phenotype (>250 substitution mutations/Mb) [4]. MMR deficiency also results in gains or losses in the repeat length of microsatellites, referred to as microsatellite instability (MSI). Cancers that possess more than 40% microsatellite variations (positive for two or more of five microsatellite markers routinely tested) are described as high frequency MSI (MSI-H). Tumours that have no MSI are microsatellite stable (MSS) and those that possess less than 40% microsatellite variations (one out of the five markers showing microsatellite instability) are low frequency MSI (MSI-L) [5]. MSI analysis is a widely used diagnostic biomarker of MMR-deficient tumours and MSI status is linked with a high prevalence of frameshift (FS) mutations that can occur because of insertion/deletion within coding microsatellites. In addition to altering downstream functions of the protein, the FS creates a new amino acid sequence that serves as a substrate for antigen processing and presentation [6], stimulating the activation of CD8+ T cells (class I) and the “helper” function of CD4+ T cells (class 11).

Cancers with a greater number of neoantigens are more prone to immune surveillance and have an increased likelihood of responding to immunotherapy [7]; higher neoantigen load is associated with overall lymphocytic infiltration, TILs, memory T cells, and survival in colorectal cancer [8, 9]. This feature supports a rationale for immunotherapy-based treatment strategies [6]. Consistent with this notion, immune checkpoint inhibitors now offer a significant therapeutic advance in the treatment of MMR-deficient cancers. Inhibitors of PD-1; for example, pembrolizumab (Keytruda) and nivolumab (Opdivo), have been approved by the Food and Drug Administration (FDA) for patients with MMR-D or MSI-H metastatic CRC based upon the significant survival benefit they provide. The CTLA-4 inhibitor ipilimumab (Yervoy), has been approved for use in combination with nivolumab for the treatment of MMR-D or MSI-H CRC patients who were previously treated with chemotherapy. Importantly, the FDA has approved the use of pembrolizumab in MMR-D/MSI-H cancers regardless of histological tumour type [10].

It is now accepted that clinical responses to immune checkpoint inhibitors require the existence of tumour neoantigens and infiltration of T cells that recognize such neoantigens. Higher neoantigen load is associated with response to CTLA-4 and PD-1 blockade in patients with melanoma and non-small-cell lung cancer [11, 12, 13]. The number of neoantigens is linked to TMB, and several large studies have confirmed that high TMB correlates with enhanced checkpoint inhibitor responses and improved overall survival in certain tumour types, such as urothelial carcinoma [14], non-small cell lung cancer [15-18] and small cell lung cancer [19].

Germano et al. recently proposed that MMR inactivation through silencing of MLH1 increases TMB and leads to “dynamic mutational profiles”, resulting in persistent renewal of neoantigens both in vitro and in vivo. This triggers immune surveillance and leads to the control of tumour growth, particularly in combination with immune checkpoint inhibition, in mouse models [20]. Similar results are observed upon silencing of MSH2 [21].

Guan et al. and Lu et al. report that MLH1 deficiency leads to cytosolic DNA release, activation of the cGAS-STING pathway and IFN-β production. Guan et al. demonstrate that MLH1 loss leads to DNA hyperexcision, RPA exhaustion, chromosomal instability and accumulation of cytosolic DNA [22]. Lu et al. report that the sensing of cytosolic DNA by the cGAS STING pathway contributes to the clinical benefit of immunotherapy in patients harboring MMR deficient tumours [23]. Together these reports suggest that abrogation of MMR activity may elicit beneficial immune activation through activation of the cGAS-STING pathway.

MLH1 and PMS2 commonly form a heterodimer; loss of MLH1 protein typically leads to concomitant loss of PMS2 protein suggesting that either or both proteins may be essential for MMR function and cGAS/STING pathway modulation.

There is therefore a biological and clinical rationale highlighting the need for inhibitors that target the PMS2 protein, a key component of DNA MMR, to reawaken an anti-tumour immune response.

Thus, the present invention provides methods for the treatment of cancer by binding to and modulating the function of the DNA MMR component PMS2 using small molecules as single agents and in combination with immunotherapy agents, other DNA damage response pathway modulators and/or standard-of-care chemotherapeutic agents.

Outside of the cancer field, triplet repeat disorders comprise over 30 human neurodegenerative and neuromuscular inherited diseases such as Huntington's disease (HD), myotonic dystrophy type 1 (DM1), fragile X syndrome type A (FRAXA), Friedreich's ataxia (FRDA), and spinocerebellar ataxias (SCAs). Such disorders are characterized by the expansion of simple repeats in genomic DNA. These unstable repeats are commonly found at different regions of several genes and their expansion can cause disease by a variety of both loss- and gain-of-function pathways, for instance through interfering with the expression or properties of the gene products, or by affecting splicing or antisense regulation. Several mechanisms including errors during DNA replication, meiotic recombination, transcription, DNA repair, and chromatin remodeling have been proposed to contribute to repeat instability, which can occur at various stages of the cell cycle. There is evidence that a functional MMR pathway is required for maintaining the stability of microsatellite sequences: for example, Msh2−/− transgenic mice bearing a copy of the human HD exon 1 (containing the CAG repeats) showed reduced expansion of the introduced (CAG)n repeats when compared with Msh2+/+HD exon 1 mice counterparts [24].

Thus, there is a further need for compounds that target components of the DNA MMR process, including PMS2, for treating triplet repeat disorders. The present invention was devised with the foregoing in mind.

### REFERENCES

1. Martin-Lopez, J. V. and R. Fishel, The mechanism of mismatch repair and the functional analysis of mismatch repair defects in Lynch syndrome. Fam Cancer, 2013. 12(2): p. 159-68.
2. Liu, D., G. Keijzers, and L. J. Rasmussen, DNA mismatch repair and its many roles in eukaryotic cells. Mutat Res, 2017. 773: p. 174-187.
3. Lynch, H. T., et al., Review of the Lynch syndrome: history, molecular genetics, screening, differential diagnosis, and medicolegal ramifications. Clin Genet, 2009. 76(1): p. 1-18.
4. Shlien, A., et al., Combined hereditary and somatic mutations of replication error repair genes result in rapid onset of ultra-hypermutated cancers. Nat Genet, 2015. 47(3): p. 257-62
5. Sehgal, R., et al., Lynch syndrome: an updated review. Genes (Basel), 2014. 5(3): p. 497-507
6. Willis, J. A., et al., Immune Activation in Mismatch Repair-Deficient Carcinogenesis: More Than Just Mutational Rate. Clin Cancer Res, 2019.
7. Gubin, M. M. and R. D. Schreiber, CANCER. The odds of immunotherapy success. Science, 2015. 350(6257): p. 158-9.
8. Kloor, M. and M. von Knebel Doeberitz, The Immune Biology of Microsatellite-Unstable Cancer. Trends Cancer, 2016. 2(3): p. 121-133.
9. Giannakis, M., et al., Genomic Correlates of Immune-Cell Infiltrates in Colorectal Carcinoma. Cell Rep, 2016. 17(4): p. 1206.
10. Lemery, S., P. Keegan, and R. Pazdur, First FDA Approval Agnostic of Cancer Site—When a Biomarker Defines the Indication. N Engl J Med, 2017. 377(15): p. 1409-1412.
11. Le, D. T., et al., PD-1 Blockade in Tumors with Mismatch-Repair Deficiency. N Engl J Med, 2015. 372(26): p. 2509-20.
12. Rizvi, N. A., et al., Cancer immunology. Mutational landscape determines sensitivity to PD-1 blockade in non-small cell lung cancer. Science, 2015. 348(6230): p. 124-8.
13. Van Allen, E. M., et al., Genomic correlates of response to CTLA-4 blockade in metastatic melanoma. Science, 2015. 350(6257): p. 207-211.
14. Rosenberg, J. E., et al., Atezolizumab in patients with locally advanced and metastatic urothelial carcinoma who have progressed following treatment with platinum-based chemotherapy: a single-arm, multicentre, phase 2 trial. Lancet, 2016. 387(10031): p. 1909-20.
15. Hellmann, M. D., et al., Genomic Features of Response to Combination Immunotherapy in Patients with Advanced Non-Small-Cell Lung Cancer. Cancer Cell, 2018. 33(5): p. 843-852 e4.
16. Rizvi, H., et al., Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol, 2018. 36(7): p. 633-641.
17. Carbone, D. P., et al., First-Line Nivolumab in Stage IV or Recurrent Non-Small-Cell Lung Cancer. N Engl J Med, 2017. 376(25): p. 2415-2426.
18. Hellmann, M. D., et al., Nivolumab plus Ipilimumab in Lung Cancer with a High Tumor Mutational Burden. N Engl J Med, 2018. 378(22): p. 2093-2104.
19. Hellmann, M. D., et al., Tumor Mutational Burden and Efficacy of Nivolumab Monotherapy and in Combination with Ipilimumab in Small-Cell Lung Cancer. Cancer Cell, 2018. 33(5): p. 853-861 e4.
20. Germano, G., et al., Inactivation of DNA repair triggers neoantigen generation and impairs tumour growth. Nature, 2017. 552(7683): p. 116-120.
21. Mandal, R., et al., Genetic diversity of tumors with mismatch repair deficiency influences anti-PD-1 immunotherapy response. Science, 2019. 364(6439): p. 485-491.
22. Guan J., et al., MLH1 deficiency-triggered DNA hyperexcision by exonuclease 1 activates the cGAS-STING pathway. Cancer Cell. 2021, 39 (1), 109-121,
23. Lu, C., et al. DNA sensing in mismatch repair-deficient tumor cells is essential for anti-tumor immunity. Cancer Cell. 2021, 39 (1), 96-108.
24. Manley, K., et al., Msh2 deficiency prevents in vivo somatic instability of the CAG repeat in Huntington disease transgenic mice. Nat Genet, 1999. 23(4): p. 471-3.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting PMS2 activity, in vitro or in vivo, said method comprising contacting a cell with an effective amount of a compound or a pharmaceutically acceptable salt, hydrate or solvate thereof as defined herein, or a pharmaceutical composition as defined herein.

According to a further aspect of the present invention, there is provided a method of treating a disease or disorder in which PMS2 activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a triplet repeat disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound, or a pharmaceutically acceptable salt, hydrate or solvate thereof, or a pharmaceutical composition as defined herein for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, or a pharmaceutical composition as defined herein for use in the treatment of a triplet repeat disorder. In a particular embodiment, the triplet repeat disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a compound, or a pharmaceutically acceptable salt, hydrate or solvate thereof, as defined herein, or a pharmaceutical composition as defined herein for use in the inhibition of PMS2 activity.

According to a further aspect of the present invention, there is provided a compound, or a pharmaceutically acceptable salt, hydrate or solvate thereof, as defined herein, or a pharmaceutical composition as defined herein for use in the treatment of a disease or disorder in which PMS2 activity is implicated.

According to a further aspect of the present invention, there is provided the use of a compound, or a pharmaceutically acceptable salt, hydrate or solvate thereof, as defined herein, or a pharmaceutical composition as defined herein in the manufacture of a medicament for the treatment of a proliferative disorder.

According to a further aspect of the present invention, there is provided the use of a compound, or a pharmaceutically acceptable salt, hydrate or solvate thereof, as defined herein, or a pharmaceutical composition as defined herein in the manufacture of a medicament for the treatment of cancer.

According to a further aspect of the present invention, there is provided the use of a compound, or a pharmaceutically acceptable salt, hydrate or solvate thereof, as defined herein, or a pharmaceutical composition as defined herein in the manufacture of a medicament for the treatment of a triplet repeat disorder. In a particular embodiment, the triplet repeat disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a use of a compound, or a pharmaceutically acceptable salt, hydrate or solvate thereof, as defined herein, or a pharmaceutical composition as defined herein in the manufacture of a medicament for the inhibition of PMS2 activity.

According to a further aspect of the present invention, there is provided a use of a compound, or a pharmaceutically acceptable salt, hydrate or solvate thereof, as defined herein, or a pharmaceutical composition as defined herein in the manufacture of a medicament for the treatment of a disease or disorder in which PMS2 activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

In the above-outlined aspects of the invention, the proliferative disorder is suitably cancer, and the cancer is suitably a human cancer. In particular, the compounds of the present invention will be useful for the treatment of any cancer in which mis-match repair inhibition and/or cGAS/STING pathway activation is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukemia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkylenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (=O) or thioxo (=S) substituents. A suitable value for a heterocyclyl group which bears 1 or 2 oxo (=O) or thioxo (=S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

The phrase “bridged ring systems” means ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

The phrase “spiro bi-cyclic ring systems” means that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to compounds, or pharmaceutically acceptable salts, hydrates or solvates thereof, having the structural Formula (I), shown below:

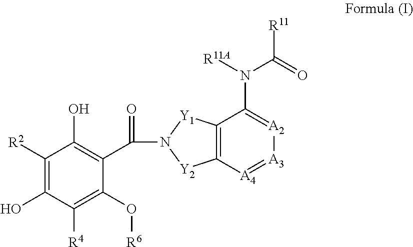

wherein
R² is hydrogen or fluoro;
R⁴ is selected from the group consisting of hydrogen, halogen, (1-6C)alkyl, (1-6C)haloalkyl, (3-6C)cycloalkyl and (3-6C)cycloalkyl(1-2C)alkyl, wherein the said (1-6C)alkyl is optionally substituted by one or more R^{5a} and the said (3-6C)cycloalkyl and (3-6C)cycloalkyl(1-2C)alkyl groups are optionally substituted with one or more R^{5b}; where each R^{5a} is independently selected from halogen or (1-4C)alkoxy and each R^{5b} is independently selected from the group consisting of halogen, (1-4C)alkyl and (1-4C)alkoxy;
R⁶ is (1-6C)alkyl, (3-8C)cycloalkyl, or a 4- to 7-membered heterocyclyl ring comprising one heteroatom selected from N, O or S,
   or a group having a structure according to formula (A) shown below:

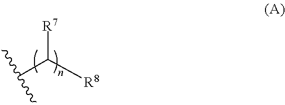

wherein
   R⁷ is hydrogen or (1-3C)alkyl;
   n is 1 or 2;
   R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂—, —C(═O)—, or —CHR_{y2a}—, where R_{y2a} is selected from halo, cyano, methyl, methoxy, CF₃, —OCF₃ or hydroxymethyl;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
R^{11A} is hydrogen or (1-3C)alkyl;
R¹¹ is a group of the formula (A) or (B) shown below:

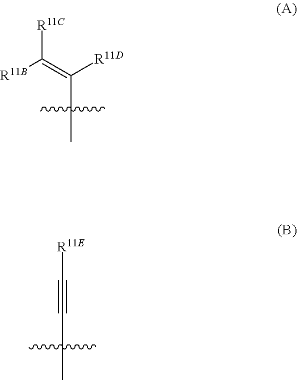

wherein:
   R^{11B} and R^{11C} are each independently selected from the group consisting of hydrogen, halo, (1-4C)alkyl, (3-6C)cycloalkyl, (3-6C)cycloalkyl-(1-2C)alkyl, wherein any (1-4C)alkyl, (3-6C)cycloalkyl or (3-6C)cycloalkyl-(1-2C)alkyl group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
   R^{11D} is hydrogen, halo or (1-3C)alkyl;
   R^{11E} is selected from the group consisting of hydrogen, halo, (1-4C)alkyl, (3-6C)cycloalkyl, (3-6C)cycloalkyl-(1-2C)alkyl, wherein any (1-4C)alkyl, (3-6C)cycloalkyl or (3-6C)cycloalkyl-(1-2C)alkyl group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
A₂ is selected from N, CH or CR¹²;
A₃ is selected from N, CH or CR¹³;
A₄ is selected from N, CH or CR¹⁴;
   with the proviso that only one or two of A₂, A₃ or A₄ can be N;
R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹² is —(CHRₚ)_{f}—Z¹²,
      wherein Rₚ is hydrogen or methyl;
      wherein f is 0 or 1; and
      Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 6-membered heterocyclyl, a 5 or 6 membered heteroaryl, —(CHR_{q})ₑ-(3-7C)cycloalkyl, —(CHR_{q})ₑ-phenyl, —(CHR_{q})ₑ-[4 to 6-membered heterocyclyl] or —(CHR_{q})ₑ-[5 or 6 membered heteroaryl], wherein R_{q} is hydrogen or methyl and e is 0 or 1;
         R²¹ and R²² are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRᵣ)_{d}-(3-7C)cycloalkyl, —(CHRᵣ)_{d}-phenyl, —(CHRᵣ)_{d}-[4 to 6-membered heterocyclyl] or —(CHRᵣ)_{d}-[5 or 6 membered heteroaryl], wherein Rᵣ is hydrogen or methyl and d is 0 or 1;
         or R²¹ and R²² are linked, such that, together with the nitrogen atom to which they are attached, they form a 4-6 membered heterocyclic ring;
         R²³ is hydrogen or (1-2C)alkyl;
         R²⁴ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₛ)_{c}-(3-7C)cycloalkyl, —(CHRₛ)_{c}-phenyl, —(CHRₛ)_{c}-[4 to 6-membered heterocyclyl] or —(CHRₛ)_{c}-[5 or 6 membered heteroaryl], wherein Rₛ is hydrogen or methyl and c is 0 or 1;
   wherein each of R²⁰, R²¹, R²², R²³ or R²⁴ any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹³ is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 6-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 6-membered heterocyclyl] or —(CHRₙ)ⱼ-[5 or 6 membered heteroaryl], wherein Rₙ is hydrogen or methyl and i is 0 or 1;
         R²⁶ and R²⁷ are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRₘ)ᵢ-(3-7C)cycloalkyl, —(CHRₘ)ᵢ-phenyl, —(CHRₘ)ᵢ-[4 to 6-membered heterocyclyl] or —(CHRₘ)ᵢ-[5 or 6 membered heteroaryl], wherein Rₙ is hydrogen or methyl and j is 0 or 1;
         or R²⁶ and R²⁷ are linked, such that, together with the nitrogen atom to which they are attached, they form a 4-6 membered heterocyclic ring;
         R²⁸ is hydrogen or (1-2C)alkyl;
         R²⁹ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 6-membered heterocyclyl] or —(CHRₗ)ₖ-[5 or 6 membered heteroaryl], wherein Rₗ is hydrogen or methyl and k is 0 or 1;
   wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹⁴ is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 6-membered heterocyclyl, a 5 or 6 membered heteroaryl, —(CHRⱼ)ₒ-(3-7C)cycloalkyl, —(CHRⱼ)ₒ-[⁴ to 6-membered heterocyclyl] or —(CHRⱼ)ₒ-[⁵ or 6 membered heteroaryl], wherein R; is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 6-membered heterocyclyl, a 5 or 6 membered heteroaryl, —(CHRᵢ)ₚ-(3-7C)cycloalkyl, —(CHRᵢ)ₚ-[4 to 6-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-6 membered heterocyclic ring;
         R³³ is hydrogen or (1-2C)alkyl;
         R³⁴ is (1-6C)alkyl, (2-6C)alkynyl, (3-7C)cycloalkyl, a carbon-linked 4 to 6-membered heterocyclyl, a 5 or 6 membered heteroaryl, —(CHRₕ)_{q}-(3-7C)cycloalkyl, —(CHRₙ)_{q}-[⁴ to 6-membered heterocyclyl] or —(CHRₙ)_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group

-L¹-X¹-Q¹
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —N(R^{14a})—C(O)—NR-^{4a}—, —SO₂N(R^{14a})—, or —N(R^{14a})SO₂—, where R^{4a} is hydrogen or (1-2C)alkyl; and
   Q¹ is selected from the group consisting of hydrogen, (1-4C)alkyl, (2-4C)alkenyl, (2-4C)alkynyl, or (3-6C)cycloalkyl.

In one aspect, the present invention also relates to compounds, or pharmaceutically acceptable salts, hydrates or solvates thereof, having the structural Formula (II), shown below:

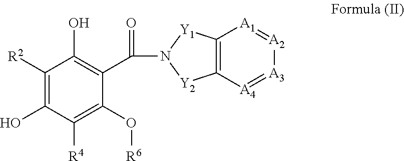

wherein
R² is hydrogen or fluoro;
R⁴ is selected from the group consisting of hydrogen, halogen, (1-6C)alkyl, (1-6C)haloalkyl, (3-6C)cycloalkyl and (3-6C)cycloalkyl(1-2C)alkyl, wherein the said (1-6C)alkyl is optionally substituted by one or more R^{b}a and the said (3-6C)cycloalkyl and (3-6C)cycloalkyl(1-2C)alkyl groups are optionally substituted with one or more R^{5b}; where each R^{5a} is independently selected from halogen or (1-4C)alkoxy and each R^{5b} is independently selected from the group consisting of halogen, (1-4C)alkyl and (1-4C)alkoxy;
R⁶ is (1-6C)alkyl, (1-6C)haloalkyl, (3-8C)cycloalkyl, or a 4- to 7-membered heterocyclyl ring comprising one heteroatom selected from N, O or S,
   or a group having a structure according to formula (A) shown below:

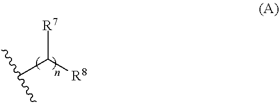

wherein
   R⁷ is hydrogen or (1-3C)alkyl;
   n is 1 or 2;
   R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂—, —C(═O)—, or —CHR_{y2a}—, where R_{y2a} is selected from halo, cyano, methyl, methoxy, CF₃, —OCF₃ or hydroxymethyl;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH, CR¹⁰ or CR¹²;
A₂ is selected from N, CH, CR¹⁰ or CR¹²;
A₃ is selected from N, CH or CR¹³;
A₄ is selected from N, CH or CR¹⁴;
   with the proviso that:
   only one or two of A₁, A₂, A₃ or A₄ can be N; and
   one of A₁ and A₂ is CR¹⁰;
R¹⁰ is a group of the formula (IIA) or (IIB) shown below:

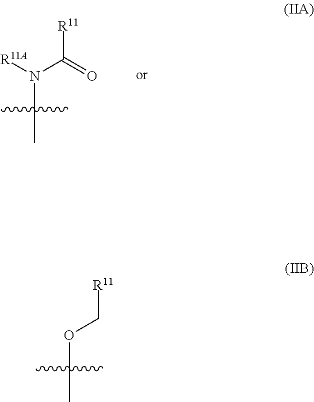

wherein [symbol] denotes the point of attachment to A₁ or A₂,
R^{11A} is hydrogen or (1-3C)alkyl;
R¹¹ is a group of the formula (A) or (B) shown below:

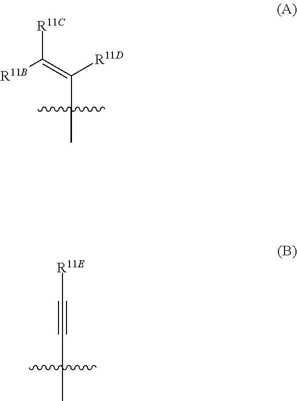

wherein:
   R^{11B} and R^{11C} are each independently selected from the group consisting of hydrogen, halo, (1-4C)alkyl, (3-6C)cycloalkyl, (3-6C)cycloalkyl-(1-2C)alkyl and —C(O)NR^{11F}R^{11G}, wherein R^{11F} and R^{11G} are each independently selected from hydrogen or (1-4C)alkyl;
      and wherein any (1-4C)alkyl, (3-6C)cycloalkyl, (3-6C)cycloalkyl-(1-2C)alkyl present in a R^{11B} and/or R^{11C} group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
   R^{11D} is selected from hydrogen, halo, (1-4C)alkyl, (3-6C)cycloalkyl, (3-6C)cycloalkyl-(1-2C)alkyl and —C(O)H, wherein any (1-4C)alkyl, (3-6C)cycloalkyl, (3-6C)cycloalkyl-(1-2C)alkyl or —C(O)H group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy or (1-3C)alkyl;
   R^{11E} is selected from the group consisting of hydrogen, halo, (1-4C)alkyl, (3-6C)cycloalkyl, (3-6C)cycloalkyl-(1-2C)alkyl, wherein any (1-4C)alkyl, (3-6C)cycloalkyl or (3-6C)cycloalkyl-(1-2C)alkyl group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹² is —(CHRₚ)_{f}—Z¹²,
      wherein Rₚ is hydrogen or methyl;
      wherein f is 0 or 1; and
      Z¹² is —OR²⁰, —NR²¹R²², —C(O)NR²¹R²² or —NR²³C(O)R²⁴
         wherein R²⁰ is (1-4C)alkyl, (3-7C)cycloalkyl, phenyl, a carbon-linked 4 to 6-membered heterocyclyl, a 5 or 6 membered heteroaryl, —(CHR_{q})ₑ-(3-7C)cycloalkyl, —(CHR_{q})ₑ-phenyl, —(CHR_{q})ₑ-[4 to 6-membered heterocyclyl] or —(CHR_{q})ₑ-[⁵ or 6 membered heteroaryl], wherein R_{q} is hydrogen or methyl and e is 0 or 1;
         R²¹ and R²² are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRᵣ)_{d}-(3-7C)cycloalkyl, —(CHRᵣ)_{d}-phenyl, —(CHRᵣ)_{d}-[4 to 6-membered heterocyclyl] or —(CHRᵣ)_{d}-[⁵ or 6 membered heteroaryl], wherein Rᵣ is hydrogen or methyl and d is 0 or 1;
         or R²¹ and R²² are linked, such that, together with the nitrogen atom to which they are attached, they form a 4-6 membered heterocyclic ring;
         R²³ is hydrogen or (1-2C)alkyl;
         R²⁴ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₛ)_{c}-(3-7C)cycloalkyl, —(CHRₛ)_{c}-phenyl, —(CHRₛ)_{c}-[4 to 6-membered heterocyclyl] or —(CHRₛ)_{c}-[5 or 6 membered heteroaryl], wherein Rₛ is hydrogen or methyl and c is 0 or 1;
wherein each of R²⁰, R²¹, R²², R²³ or R²⁴ or any ring formed when R²¹ and R²² are linked, is optionally substituted with one or more R^{a}; or
R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³
      wherein Rₒ is hydrogen or methyl;
      wherein h is 0 or 1; and
      Z¹³ is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 6-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 6-membered heterocyclyl] or —(CHRₙ)ᵢ-[5 or 6 membered heteroaryl], wherein Rₙ is hydrogen or methyl and i is 0 or 1;
         R²⁶ and R²⁷ are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRₘ)ⱼ-(3-7C)cycloalkyl, —(CHRₘ)ᵢ-phenyl, —(CHRₘ)ᵢ-[4 to 6-membered heterocyclyl] or —(CHRₘ)ᵢ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-6 membered heterocyclic ring;
         R²⁸ is hydrogen or (1-2C)alkyl;
         R²⁹ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 6-membered heterocyclyl] or —(CHRₗ)ₖ-[5 or 6 membered heteroaryl], wherein Rₗ is hydrogen or methyl and k is 0 or 1;
   wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹⁴ is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 6-membered heterocyclyl, a 5 or 6 membered heteroaryl, —(CHRⱼ)ₒ-(3-7C)cycloalkyl, —(CHRⱼ)ₒ-[⁴ to 6-membered heterocyclyl] or —(CHRⱼ)ₒ-[⁵ or 6 membered heteroaryl], wherein R; is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 6-membered heterocyclyl, a 5 or 6 membered heteroaryl, —(CHRᵢ)ₚ-(3-7C)cycloalkyl, —(CHRᵢ)ₚ-[4 to 6-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-6 membered heterocyclic ring;
         R³³ is hydrogen or (1-2C)alkyl;
         R³⁴ is (1-6C)alkyl, (2-6C)alkynyl, (3-7C)cycloalkyl, a carbon-linked 4 to 6-membered heterocyclyl, a 5 or 6 membered heteroaryl, —(CHRₕ)_{q}-(3-7C)cycloalkyl, —(CHR_{b})_{q}-[⁴ to 6-membered heterocyclyl] or —(CHR_{b})_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is
   optionally substituted with one or more R^{a};
and wherein each R^{a} is independently selected from the group consisting of oxo, halogen, cyano, hydroxy, (1-4C)alkyl, or a group

-L¹-X¹-Q¹
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —N(R^{14a})—C(O)—NR^{14a}, —SO₂N(R^{14a})—, or —N(R^{14a})SO₂—, where R^{14a} is hydrogen or (1-2C)alkyl; and
   Q¹ is selected from the group consisting of hydrogen, (1-4C)alkyl, (2-4C)alkenyl, (2-4C)alkynyl, or (3-6C)cycloalkyl.

Particular compounds of the invention include, for example, compounds of the Formula (I) or Formula (II), or pharmaceutically acceptable salts, hydrates and/or solvates thereof, wherein, unless otherwise stated, each of R², R⁴ (and R^{5a} and R^{5b}), R⁶ (and R⁷, n, R⁸, m, and R⁹), R¹¹ (and R^{11B}, R^{11C} and R^{11d}), R^{11A}, Y₁, Y₂, A₂ (and R¹²), A₃ (and R¹³), A₄ (and R¹⁴) and R^{a}, and any associated substituent groups, has any of the meanings defined hereinbefore or in any of paragraphs (1) to (102) hereinafter:
(1) R² is fluoro.
(2) R² is hydrogen.
(3) R⁴ is selected from the group consisting of hydrogen, halogen, (1-4C)alkyl, (1-4C)haloalkyl, (3-6C)cycloalkyl and (3-6C)cycloalkyl(1-2C)alkyl, wherein the said (1-4C)alkyl is optionally substituted by one or more R^{5a} and the said (3-6C)cycloalkyl and (3-6C)cycloalkyl(1-2C)alkyl groups are optionally substituted with one or more R^{5b}, wherein R^{5a} and R^{5b} are each as defined hereinbefore or R^{5a} is as defined in paragraph (14), (15) or (16) below and/or R^{5b} is as defined in paragraph (17), (18), (19) or (20) below.
(4) R⁴ is selected from the group consisting of hydrogen, halogen, (1-4C)alkyl, (1-4C)haloalkyl, (3-5C)cycloalkyl and (3-5C)cycloalkyl(1-2C)alkyl, wherein the said (1-4C)alkyl is optionally substituted by one, two or three R^{5a} and the said (3-5C)cycloalkyl and (3-5C)cycloalkyl(1-2C)alkyl groups are optionally substituted with one, two or three R^{5b}, wherein R^{5a} and R^{5b} are each as defined hereinbefore or R^{5a} is as defined in paragraph (14), (15) or (16) below and/or R^{5b} is as defined in paragraph (17), (18), (19) or (20) below.
(5) R⁴ is selected from the group consisting of hydrogen, halogen, (1-4C)alkyl, (1-4C)haloalkyl, (3-5C)cycloalkyl and (3-5C)cycloalkyl(1C)alkyl, wherein the said (1-4C)alkyl is optionally substituted by one, two or three R^{5a} and the said (3-5C)cycloalkyl and (3-5C)cycloalkyl(1C)alkyl groups are optionally substituted with one, two or three R^{5b}, wherein R^{5a} and R^{5b} are each as defined hereinbefore or R^{5a} is as defined in paragraph (14), (15) or (16) below and/or R^{5b} is as defined in paragraph (17), (18), (19) or (20) below.
(6) R⁴ is selected from the group consisting of hydrogen, halogen, (1-4C)alkyl, (1-2C)haloalkyl and (3-5C)cycloalkyl, wherein the said (1-4C)alkyl is optionally substituted by one or two R⁵² and the said (3-5C)cycloalkyl group is optionally substituted with one or two R^{5b}, wherein R^{5a} and R^{5b} are each as defined hereinbefore or R^{5a} is as defined in paragraph (14), (15) or (16) below and/or R^{5b} is as defined in paragraph (17), (18), (19) or (20) below.
(7) R⁴ is selected from the group consisting of hydrogen, fluoro, chloro, (1-4C)alkyl, CH₂F, CHF₂, CF₃, cyclopropyl and cyclobutyl, wherein the said (1-4C)alkyl is optionally substituted by one R^{5a} and the said cyclopropyl and cyclobutyl groups are optionally substituted with one R^{5b}, wherein R^{5a} and R^{5b} are each as defined hereinbefore or R^{5a} is as defined in paragraph (14), (15) or (16) below and/or R^{5b} is as defined in paragraph (17), (18), (19) or (20) below.
(8) R⁴ is selected from the group consisting of hydrogen, fluoro, chloro, methyl, ethyl, isopropyl, CH₂F, CHF₂, CF₃, cyclopropyl and cyclobutyl.
(9) R⁴ is selected from the group consisting of hydrogen, methyl, ethyl, CHF₂, CF₃, isopropyl, cyclopropyl and cyclobutyl.
(10) R⁴ is selected from the group consisting of hydrogen, methyl, ethyl, CHF₂, CF₃ and cyclopropyl.
(11) R⁴ is selected from the group consisting of hydrogen and methyl.
(12) R⁴ is hydrogen.
(13) R⁴ is methyl.
(14) Each R^{5a} is independently selected from halogen or (1-3C)alkoxy.
(15) Each R^{5a} is independently selected from fluoro, chloro or (1-2C)alkoxy.
(16) Each R^{5a} is independently selected from fluoro, chloro or methoxy.
(17) Each R^{5b} is independently selected from the group consisting of halogen, (1-3C)alkyl and (1-3C)alkoxy.
(18) Each R^{5b} is independently selected from the group consisting of halogen, (1-2C)alkyl and (1-2C)alkoxy.
(19) Each R^{5b} is independently selected from the group consisting of fluoro, chloro, (1-2C)alkyl and (1-2C)alkoxy.
(20) Each R^{5b} is independently selected from the group consisting of fluoro, chloro, methyl and methoxy.
(21) R⁶ is (1-6C)alkyl, (3-6C)cycloalkyl, or a 4- to 6-membered heterocyclyl ring comprising one heteroatom selected from N, O or S,
   or a group having a structure according to formula (A) shown below:

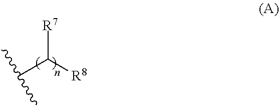

wherein n, R⁷ and R⁸ are each as defined hereinbefore, or n is as defined in paragraph (31) or (32) below, R⁷ is as defined in paragraph (27), (28), (29) or (30) below, and R⁸ is as defined in paragraph (33), (34), (35), (36), (37), (38), (39), (40), (41), (42), (43), (44), or (107) below.
(22) R⁶ is (1-4C)alkyl, (3-6C)cycloalkyl, or a 4- to 6-membered heterocyclyl ring comprising one heteroatom selected from N, O or S,
   or a group having a structure according to formula (A) shown below:

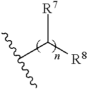

wherein n, R⁷ and R⁸ are each as defined herein, or n is as defined in paragraph (31) or (32) below, R⁷ is as defined in paragraph (27), (28), (29) or (30) below, and R⁸ is as defined in paragraph (33), (34), (35), (36), (37), (38), (39), (40), (41), (42), (43), (44), or (107) below.
(23) R⁶ is (1-4C)alkyl, (3-6C)cycloalkyl, or a 4- to 6-membered heterocyclyl ring comprising one heteroatom selected from O or S,
   or a group having a structure according to formula (A) shown below:

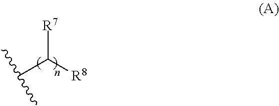

wherein n, R⁷ and R⁸ are each as defined herein, or n is as defined in paragraph (31) or (32) below, R⁷ is as defined in paragraph (27), (28), (29) or (30) below, and R⁸ is as defined in paragraph (33), (34), (35), (36), (37), (38), (39), (40), (41), (42), (43), (44), or (107) below.
(24) R⁶ is (1-4C)alkyl or a 5- or 6-membered heterocyclyl ring comprising one heteroatom selected from O or S; or a group having a structure according to formula (A) shown below:

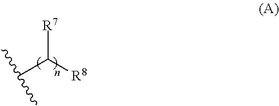

wherein n, R⁷ and R⁸ are each as defined herein, or n is as defined in paragraph (31) or (32) below, R⁷ is as defined in paragraph (27), (28), (29) or (30) below, and R⁸ is as defined in paragraph (33), (34), (35), (36), (37), (38), (39), (40), (41), (42), (43), (44), or (107) below.
(25) R⁶ is (1-3C)alkyl, tetrahydrofuran, tetrahydropyran or a group having a structure according to formula (A) shown below:

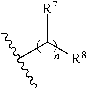

wherein n, R⁷ and R⁸ are each as defined herein, or n is as defined in paragraph (31) or (32) below, R⁷ is as defined in paragraph (27), (28), (29) or (30) below, and R⁸ is as defined in paragraph (33), (34), (35), (36), (37), (38), (39), (40), (41), (42), (43), (44) or (107) below.
(26) R⁶ is a group having a structure according to formula (A) shown below:

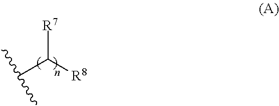

wherein n, R⁷ and R⁸ are each as defined herein, or n is as defined in paragraph (31) or (32) below, R⁷ is as defined in paragraph (27), (28), (29) or (30) below, and R³ is as defined in paragraph (33), (34), (35), (36), (37), (38), (39), (40), (41), (42), (43), (44), or (107) below.
(27) R⁷ is hydrogen or (1-2C)alkyl.
(28) R⁷ is hydrogen or methyl.
(29) R⁷ is hydrogen.
(30) R⁷ is methyl.
(31) n is 1.
(32) n is 2.
(33) R⁸ is (3-8C)cycloalkyl, aryl, 4- to 6-membered heterocyclyl, or 5- or 6-membered heteroaryl, each of which is optionally substituted with one or more R⁹, wherein R⁹ is as defined herein.
(34) R⁸ is (3-8C)cycloalkyl, phenyl, 4- to 6-membered heterocyclyl, or 5- or 6-membered heteroaryl, each of which is optionally substituted with one or more R⁹, wherein R⁹ is as defined herein.
(35) R⁸ is (3-6C)cycloalkyl, phenyl, 4- to 6-membered heterocyclyl, or 5- or 6-membered heteroaryl, each of which is optionally substituted with one or more R⁹, wherein R⁹ is as defined herein.
(36) R⁸ is 5- or 6-membered cycloalkyl, phenyl, 5- or 6-membered heterocyclyl, or 6-membered heteroaryl, each of which is optionally substituted with one or more R⁹, wherein R⁹ is as defined herein.
(37) R⁸ is cyclohexyl, phenyl, 5- or 6-membered heterocyclyl, or 6-membered heteroaryl, each of which is optionally substituted with one or more R⁹, wherein R⁹ is as defined herein.
(38) R⁸ is cyclohexyl, phenyl, tetrahydrofuran, tetrahydropyran, or pyridyl, each of which is optionally substituted with one or more R⁹, wherein R⁹ is as defined herein.
(39) R⁸ is cyclohexyl, phenyl, or pyridyl, each of which is optionally substituted with one or more R⁹, wherein R⁹ is as defined herein.
(40) R⁸ has any one of the following structures:

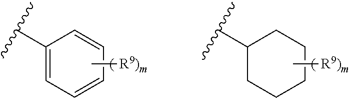

wherein R⁹ is as defined herein before (or as in any one of paragraphs (48), (49), (50) or (51) below) and each m is independently 0, 1, 2 or 3 (or as defined in paragraph (45), (46) or (47) below.
(41) R⁸ has any one of the following structures:

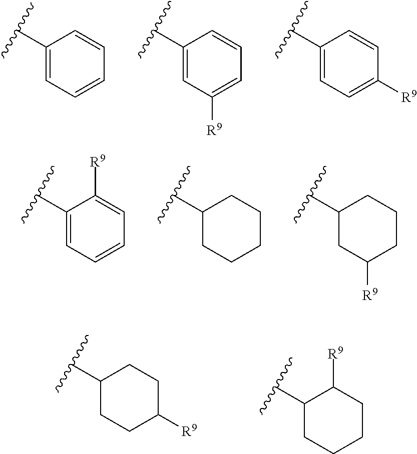

wherein R⁹ is as defined hereinbefore or as in any one of paragraphs (48), (49), (50) or (51) below.
(42) R⁸ has any one of the following structures:

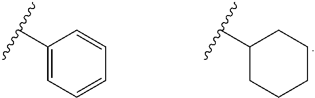 [SMILES: *c1ccccc1 ; *C1CCCCC1]

(43) R⁸ is cyclohexyl.
(44) R⁸ is phenyl.
(45) Each m is independently 0, 1 or 2.
(46) Each m is independently 0 or 1.
(47) Each m is 0.
(48) Each R⁹ is independently selected from the group consisting of hydroxy, cyano, halogen, (1-2C)alkyl, (1-2C)alkoxy, (1-2C)haloalkyl, or (1-2C)haloalkoxy.
(49) Each R⁹ is independently selected from the group consisting of halogen, (1-2C)alkyl, (1-2C)alkoxy, (1-2C)haloalkyl, or (1-2C)haloalkoxy.
(50) Each R⁹ is independently selected from the group consisting of halogen, methyl, methoxy, trifluoromethyl, or trifluoromethoxy.
(51) Each R⁹ is independently selected from the group consisting of fluoro, chloro, methyl, methoxy, trifluoromethyl, or trifluoromethoxy.
(52) Y₁ is —CH₂—, —C(═O)—, or —CHR_{y2a}—, where R_{y2a} is selected from halo, methyl, methoxy, CF₃, —OCF₃ or hydroxymethyl.
(53) Y₁ is —CH₂—.
(54) Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, methyl, methoxy, CF₃, —OCF₃ or hydroxymethyl, and R_{y2b} is selected from halo, cyano, hydroxy, methyl, methoxy, CF₃, —OCF₃ or hydroxymethyl.
(55) Y₂ is —CH₂—, —CHR_{y2a}—, —CH₂—CH₂—, —CH₂-CHR_{y2b}-, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl.
(56) Y₂ is —CH₂— or —CH₂—CH₂—.
(57) Y₂ is —CH₂—.
(58) Y₁ is —CH₂— and Y₂ is —CH₂—.
(59) R^{11A} is hydrogen, methyl or ethyl.
(60) R^{11A} is hydrogen or methyl.
(61) R^{11A} is hydrogen.
(62) R¹¹ is a group of the formula (A) or (B) shown below:

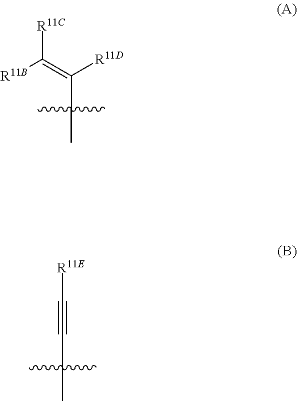

wherein R¹ 1, R^{11C}, R^{11D} or R^{11E} are each as defined herein or:
   R^{11B} and R^{11C} are as defined in any one of paragraphs (65), (66), (67), (68), (69), (116), (117), (118), (119) or (120) below;
   R^{11D} is as defined in any one of paragraphs (70), (71), (72), (73), (121), (122), (123) or (124) below;
   R^{11E} is as defined in any one of paragraphs (74), (75), (76), (77), (78), (79) or (80) below; (63) R¹¹ is a group of the formula (A) shown below:

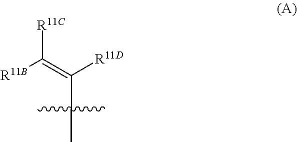

wherein R^{11B} Ru^{11C} or R^{11D} are each as defined herein or R^{11B} and Ru¹¹ are as defined in any one of paragraphs (65), (66), (67), (68), (69), (116), (117), (118), (119) or (120) below; and
R^{11D} is as defined in any one of paragraphs (70), (71), (72), (73), (121), (122), (123) or (124) below;
(64) R¹¹ is a group of the formula (B) shown below:

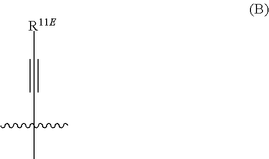

wherein R^{11E} is as defined herein or R^{11E} is as defined in any one of paragraphs (74), (75), (76), (77), (78), (79) or (80) below;
(65) R^{11B} and R^{11C} are each independently selected from the group consisting of hydrogen, halo, (1-4C)alkyl, wherein any (1-4C)alkyl group is optionally substituted with halo, hydroxy, cyano, [(1-4C)alkyl]amino, di-[(1-4C)alkyl]amino, (1-4C)alkoxy or a 4, 5 or 6-membered N-linked heterocycle which is optionally further substituted by one or more substituents selected from halo, hydroxy, cyano, (1-4C)alkyl, or (1-4C)alkoxy.
(66) R^{11B} and R^{11C} are each independently selected from the group consisting of hydrogen, halo, (1-2C)alkyl, wherein any (1-2C)alkyl group is optionally substituted with halo, hydroxy, cyano, [(1-2C)alkyl]amino, di-[(1-2C)alkyl]amino, (1-2C)alkoxy, or a 4, 5 or 6-membered N-linked heterocycle, which is optionally further substituted by one or more substituents selected from halo, hydroxy, cyano, (1-4C)alkyl, or (1-4C)alkoxy.
(67) One of R^{11B} and R^{11C} is hydrogen and the other is selected from the group consisting of hydrogen, halo, (1-2C)alkyl, wherein any (1-2C)alkyl group is optionally substituted with halo, hydroxy, cyano, [(1-2C)alkyl]amino, di-[(1-2C)alkyl]amino, (1-2C)alkoxy or a 4, 5 or 6-membered N-linked heterocycle, which is optionally further substituted by one or more substituents selected from halo, hydroxy, cyano, (1-2C)alkyl, or (1-2C)alkoxy.
(68) One of R^{11B} and R^{11C} is hydrogen and the other is selected from the group consisting of hydrogen, fluoro, (1-2C)alkyl, wherein any (1-2C)alkyl group is optionally substituted with halo, [(1-2C)alkyl]amino, di-[(1-2C)alkyl]amino or a 4, 5 or 6-membered N-linked heterocycle, which is optionally further substituted by one or more substituents selected from halo, hydroxy, cyano, (1-4C)alkyl, or (1-4C)alkoxy.
(69) One of R^{11B} and R^{11C} is hydrogen and the other is selected from the group consisting of hydrogen, fluoro, (1-2C)alkyl, wherein any (1-2C)alkyl group is optionally substituted with chloro, fluoro, di-[(1-2C)alkyl]amino or a 5 or 6-membered N-linked heterocycle, which is optionally further substituted by one or more substituents selected from chloro, fluoro, hydroxy, (1-2C)alkyl, or (1-2C)alkoxy.
(70) R^{11D} is hydrogen, halo or (1-2C)alkyl.
(71) R^{11D} is hydrogen, chloro, fluoro, methyl or ethyl.
(72) R^{11D} is hydrogen, fluoro or methyl.
(73) R^{11D} is hydrogen or fluoro.
(74) R^{11E} is selected from the group consisting of hydrogen, chloro, fluoro, (1-4C)alkyl, (4-6C)cycloalkyl, (4-6C)cycloalkyl-(1-2C)alkyl, wherein any (1-4C)alkyl, (4-6C)cycloalkyl or (4-6C)cycloalkyl-(1-2C)alkyl group is optionally substituted with one or more substituents selected from chloro, fluoro, hydroxy, cyano, [(1-4C)alkyl]amino, di-[(1-4C)alkyl]amino, (1-4C)alkoxy, (4-6C)cycloalkoxy, phenyl or 4- to 6-membered heterocyclyl; and wherein any phenyl or 4- to 6-membered heterocyclic ring is optionally further substituted by one or more substituents selected from chloro, fluoro, hydroxy, cyano, (1-4C)alkyl, (1-4C)alkoxy, (4-6C)cycloalkyl or (4-6C)cycloalkoxy.
(75) R^{11E} is selected from the group consisting of hydrogen, (1-4C)alkyl, (5-6C)cycloalkyl, (5-6C)cycloalkyl-(1-2C)alkyl, wherein any (1-4C)alkyl, (5-6C)cycloalkyl or (5-6C)cycloalkyl-(1-2C)alkyl group is optionally substituted with one or more substituents selected from chloro, fluoro, hydroxy, cyano, [(1-4C)alkyl]amino, di-[(1-4C)alkyl]amino, (1-4C)alkoxy, (4-6C)cycloalkoxy, phenyl or 4- to 6-membered heterocyclyl; and wherein any phenyl or 4- to 6-membered heterocyclic ring is optionally further substituted by one or more substituents selected from chloro, fluoro, hydroxy, cyano, (1-4C)alkyl, (1-4C)alkoxy, (4-6C)cycloalkyl or (4-6C)cycloalkoxy.
(76) R^{11E} is selected from the group consisting of hydrogen or (1-4C)alkyl, wherein any (1-4C)alkyl group is optionally substituted with one or more substituents selected from chloro, fluoro, hydroxy, cyano, [(1-4C)alkyl]amino, di-[(1-4C)alkyl]amino, (1-4C)alkoxy, (4-6C)cycloalkoxy, phenyl or 4- to 6-membered heterocyclyl; and wherein any phenyl or 4- to 6-membered heterocyclic ring is optionally further substituted by one or more substituents selected from chloro, fluoro, hydroxy, cyano, (1-4C)alkyl, (1-4C)alkoxy, (4-6C)cycloalkyl or (4-6C)cycloalkoxy.
(77) R^{11E} is (1-4C)alkyl and is optionally substituted with halo, hydroxy, cyano, [(1-4C)alkyl]amino, di-[(1-4C)alkyl]amino, (1-4C)alkoxy or a 4, 5 or 6-membered N-linked heterocycle which is optionally further substituted by one or more substituents selected from halo, hydroxy, cyano, (1-4C)alkyl or (1-4C)alkoxy.
(78) R^{11E} is (1-4)alkyl.
(79) R^{11E} is methyl or ethyl.
(80) R^{11E} is methyl.
(81) A₂ is selected from N, CH or CR¹²;
   A₃ is selected from N, CH or CR¹³;
   A₄ is selected from N, CH or CR¹⁴;
   with the proviso that only one of A₂, A₃ or A₄ can be N;
   and wherein R¹², R¹³ or R¹⁴ are as defined hereinbefore, or
      R¹² is as defined in any one of paragraphs (89), (90), (91) or (92);
      R¹³ is as defined in any one of paragraphs (93), (94) or (95); and
      R¹⁴ is as defined in any one of paragraphs (96), (97) or (98).
(82) A₂ is selected from N, CH or CR¹²;
   A₃ is selected from N, CH or CR¹³;
   A₄ is selected from N or CH;
   with the proviso that only one of A₂, A₃ or A₄ can be N;
   and wherein R¹² and R¹³ are as defined hereinbefore, or
      R¹² is as defined in any one of paragraphs (89), (90), (91) or (92); and
      R¹³ is as defined in any one of paragraphs (93), (94) or (95).
(83) A₂ is selected from N or CH;
   A₃ is selected from N, CH or CR¹³;
   A₄ is selected from N or CH;
   with the proviso that only one of A₂, A₃ or A₄ can be N;
   and wherein R¹³ is as defined hereinbefore, or R¹³ is as defined in any one of paragraphs (93), (94) or (95).
(84) A₂ is CH;
   A₃ is CH or CR¹³;
   A₄ is CH;
   and wherein R¹³ is as defined hereinbefore, or R¹³ is as defined in any one of paragraphs (93), (94) or (95)
(85) A₂ is CH;
   A₃ is CH;
   A₄ is CH;
(86) A₂ is CH or CR¹²;
   A₃ is CH;
   A₄ is CH;
   and wherein R¹² is as defined hereinbefore, or R¹² is as defined in any one of paragraphs (89), (90), (91) or (92);
(87) A₂ is CH;
   A₃ is CH;
   A₄ is CH or CR¹⁴;
   and wherein R¹⁴ is as defined hereinbefore, or R¹⁴ is as defined in any one of paragraphs (96), (97) or (98).
(88) A₂, A₃ or A₄ are all CH.
(89) R¹² is selected from halo, (1-2C)alkyl, (1-2C)alkoxy, wherein any (1-2C)alkyl or (1-2C)alkoxy is optionally substituted by one or more halo or (1-2C)alkoxy, or
   R¹² is —(CH Rₚ)_{f}-Z¹²,
   wherein Rₚ is hydrogen or methyl;
   wherein f is 0 or 1; and
   Z¹² is —OR²⁰, —NR²¹R²², —C(O)NR²¹R²² or —NR²³C(O)R²⁴;
   wherein R²⁰ is (1-3C)alkyl, (4-6C)cycloalkyl, phenyl, a carbon-linked 4 to 6-membered heterocyclyl, a 5 or 6 membered heteroaryl, —(CHR_{q})ₑ-(4-6C)cycloalkyl, —(CHR_{q})ₑ-phenyl, —(CHR_{q})ₑ-[4 to 6-membered heterocyclyl] or —(CHR_{q})ₑ-[5 or 6 membered heteroaryl], wherein R_{q} is hydrogen or methyl and e is 0 or 1;
   R²¹ and R²² are each independently selected from hydrogen, (1-4C)alkyl, (2-4C)alkanoyl, (4-6C)cycloalkyl, phenyl, a carbon-linked 4 to 6-membered heterocyclyl, a 5 or 6 membered heteroaryl, —(CHRᵣ)_{d}-(3-7C)cycloalkyl, —(CHRᵣ)_{d}-phenyl, —(CHRᵣ)_{d}-[4 to 6-membered heterocyclyl] or —(CHRᵣ)_{d}-[5 or 6 membered heteroaryl], wherein Rᵣ is hydrogen or methyl and d is 0 or 1;
   or R²¹ and R²² are linked, such that, together with the nitrogen atom to which they are attached, they form a 4-6 membered heterocyclic ring;
   R²³ is hydrogen or (1-2C)alkyl;
   R²⁴ is (1-4C)alkyl, (2-4C)alkynyl, (4-6C)cycloalkyl, phenyl, a carbon-linked 4 to 6-membered heterocyclyl, a 5 or 6 membered heteroaryl, —(CHRₛ)_{c}-(4-6C)cycloalkyl, —(CHRₛ)_{c}-phenyl, —(CHRₛ)_{c}-[4 to 6-membered heterocyclyl] or —(CHRₛ)_{c}-[5 or 6 membered heteroaryl], wherein Rₛ is hydrogen or methyl and c is 0 or 1;
   wherein each of R²⁰, R²¹, R²², R²³ or R²⁴ or any ring formed when R²¹ and R²² are linked, is optionally substituted with one or more R^{a}.
(90) R¹² is selected from halo, (1-2C)alkyl, (1-2C)alkoxy, wherein any (1-2C)alkyl or (1-2C)alkoxy is optionally substituted by one or more halo or (1-2C)alkoxy, or
   R¹² is —(CH Rₚ)_{f}-Z¹²,
   wherein Rₚ is hydrogen or methyl;
   wherein f is 0 or 1; and
   Z¹² is —OR²⁰, —NR²¹R²², —C(O)NR²¹R²² or —NR²³C(O)R²⁴;
   wherein R²⁰ is (1-2C)alkyl, (5-6C)cycloalkyl, phenyl, a carbon-linked 5 to 6-membered heterocyclyl, a 5 or 6 membered heteroaryl, —(CHR_{q})ₑ-(5-6C)cycloalkyl, —(CHR_{q})ₑ-phenyl, —(CHR_{q})ₑ-[5 to 6-membered heterocyclyl] or —(CHR_{q})ₑ-[5 or 6 membered heteroaryl], wherein R_{q} is hydrogen or methyl and e is 0 or 1;
R²¹ and R²² are each independently selected from hydrogen, (1-2C)alkyl, (2-3C)alkanoyl, (5-6C)cycloalkyl, phenyl, a carbon-linked 5 to 6-membered heterocyclyl, a 5 or 6 membered heteroaryl, —(CHRᵣ)_{d}-(5-6C)cycloalkyl, —(CHRᵣ)_{d}-phenyl, —(CHRᵣ)_{d}-[5 to 6-membered heterocyclyl] or —(CHRᵣ)_{d}-[5 or 6 membered heteroaryl], wherein Rᵣ is hydrogen or methyl and d is 0 or 1;
or R²¹ and R²² are linked, such that, together with the nitrogen atom to which they are attached, they form a 5-6 membered heterocyclic ring;
R²³ is hydrogen or (1-2C)alkyl;
R²⁴ is (1-2C)alkyl, (2-3C)alkynyl, (5-6C)cycloalkyl, phenyl, a carbon-linked 5 to 6-membered heterocyclyl, a 5 or 6 membered heteroaryl, —(CHRₛ)_{c}-(5-6C)cycloalkyl, —(CHRₛ)_{c}-phenyl, —(CHRₛ)_{c}-[5 to 6-membered heterocyclyl] or —(CHRₛ)_{c}-[5 or 6 membered heteroaryl], wherein Rₛ is hydrogen or methyl and c is 0 or 1;
wherein each of R²⁰, R²¹, R²², R²³ or R²⁴ or any ring formed when R²¹ and R²² are linked, is optionally substituted with one or more R^{a}.
(91) R¹² is selected from halo, (1-2C)alkyl, (1-2C)alkoxy, wherein any (1-2C)alkyl or (1-2C)alkoxy is optionally substituted by one or more fluoro or (1-2C)alkoxy.
(92) R¹² is selected from fluoro, (1-2C)alkyl, (1-2C)alkoxy.
(93) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
   wherein Rₒ is hydrogen or methyl;
   wherein h is 0 or 1; and
   Z¹³ is —OR²⁵, —NR²⁵R²⁷, —C(O)NR²⁶R²⁷ or —NR²⁸C(O)R²⁹;
   wherein R²⁵ is (1-3C)alkyl, (4-6C)cycloalkyl, phenyl, a carbon-linked 4 to 6-membered heterocyclyl, a 5 or 6 membered heteroaryl, —(CHRₙ)ᵢ-(4-6C)cycloalkyl, —(CHRₙ)ᵢ-phenyl, —(CHRₙ)ᵢ-[4 to 6-membered heterocyclyl] or —(CHRₙ)ᵢ-[5 or 6 membered heteroaryl], wherein Rₙ is hydrogen or methyl and i is 0 or 1;
   R²⁶ and R²⁷ are each independently selected from hydrogen, (1-4C)alkyl, (2-4C)alkanoyl, (4-6C)cycloalkyl, phenyl, a carbon-linked 4 to 6-membered heterocyclyl, a 5 or 6 membered heteroaryl, —(CHRₘ)ᵢ-(4-6C)cycloalkyl, —(CHRₘ)ⱼ-phenyl, —(CHRₘ)ⱼ-[⁴ to 6-membered heterocyclyl] or —(CHRₘ)ⱼ-[5 or 6 membered heteroaryl], wherein Rₘ is hydrogen or methyl and j is 0 or 1;
   or R²⁶ and R²⁷ are linked, such that, together with the nitrogen atom to which they are attached, they form a 4-6 membered heterocyclic ring;
   R²⁸ is hydrogen or (1-2C)alkyl;
   R²¹ is (1-4C)alkyl, (2-4C)alkynyl, (4-6C)cycloalkyl, phenyl, a carbon-linked 4 to 6-membered heterocyclyl, a 5 or 6 membered heteroaryl, —(CHRₗ)ₖ-(4-6C)cycloalkyl, —(CHRₗ)ₖ-phenyl, —(CHRₗ)ₖ-[4 to 6-membered heterocyclyl] or —(CHRₗ)ₖ-[5 or 6 membered heteroaryl], wherein Rₗ is hydrogen or methyl and k is 0 or 1;
wherein each of R²⁵, R²⁶, R²⁷, R²⁸ or R²⁹ or any ring formed when R²⁶ and R²⁷ are linked, is optionally substituted with one or more R^{a}.
(94) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
   wherein Rₒ is hydrogen or methyl;
   wherein h is 0 or 1; and
   Z¹³ is —OR²⁵, —NR²⁶R²⁷, —C(O)NR²⁶R²⁷ or —NR²⁸C(O)R²⁹;
   wherein R²⁵ is (1-2C)alkyl, (5-6C)cycloalkyl, phenyl, a carbon-linked 5 to 6-membered heterocyclyl, a 5 or 6 membered heteroaryl, —(CHRₙ)ᵢ-(5-6C)cycloalkyl, —(CHRₙ)ᵢ-phenyl, —(CHRₙ)ᵢ-[5 to 6-membered heterocyclyl] or —(CHRₙ)ᵢ-[5 or 6 membered heteroaryl], wherein Rₐ is hydrogen or methyl and i is 0 or 1;
   R²⁶ and R²⁷ are each independently selected from hydrogen, (1-2C)alkyl, (2-3C)alkanoyl, (5-6C)cycloalkyl, phenyl, a carbon-linked 5 to 6-membered heterocyclyl, a 5 or 6 membered heteroaryl, —(CHRₘ)ⱼ-(5-6C)cycloalkyl, —(CHRₘ)ⱼ-phenyl, —(CHRₘ)ⱼ-[⁵ to 6-membered heterocyclyl] or —(CHRₘ)ⱼ-[5 or 6 membered heteroaryl], wherein Rₘ is hydrogen or methyl and j is 0 or 1;
   or R²⁶ and R²⁷ are linked, such that, together with the nitrogen atom to which they are attached, they form a 5-6 membered heterocyclic ring;
   R²⁸ is hydrogen or (1-2C)alkyl;
   R²⁹ is (1-2C)alkyl, (2-3C)alkynyl, (5-6C)cycloalkyl, phenyl, a carbon-linked 5 to 6-membered heterocyclyl, a 5 or 6 membered heteroaryl, —(CHRₗ)ₖ-(5-6C)cycloalkyl, —(CHRₗ)ₖ-phenyl, —(CHRₗ)ₖ-[5 to 6-membered heterocyclyl] or —(CHRₗ)ₖ-[5 or 6 membered heteroaryl], wherein Rₒ is hydrogen or methyl and k is 0 or 1;
wherein each of R²⁵, R²⁶, R²⁷, R²⁸ or R²⁹ or any ring formed when R²⁶ and R²⁷ are linked, is optionally substituted with one or more R^{a}.
(95) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHRₒ)ₙ-Z¹³,
   wherein Rₒ is hydrogen or methyl;
   wherein h is 0 or 1; and
   Z¹² is —OR²⁵, —NR²⁶R²⁷, or —C(O)NR²⁶R²⁷;
   wherein R²⁵ is (1-4C)alkyl, a carbon-linked 4 to 8-membered heterocyclyl, or —(CHRₙ)ᵢ-[4 to 8-membered heterocyclyl], wherein Rₙ is hydrogen or methyl and i is 0 or 1;
   R²⁶ and R²⁷ are each independently selected from hydrogen, (1-6C)alkyl, a carbon-linked 4 to 8-membered heterocyclyl, or —(CHRₘ)ᵢ-[4 to 8-membered heterocyclyl], wherein Rₘ is hydrogen or methyl and j is 0 or 1;
   or R²⁶ and R²⁷ are linked, such that, together with the nitrogen atom to which they are attached, they form a 4-8 membered heterocyclic ring;
   wherein each of R²⁵, R²⁶, R²⁷ or any ring formed when R²⁶ and R²⁷ are linked, is optionally substituted with one or more R^{a}.
(96) R¹⁴ is selected from halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
   wherein Rₖ is hydrogen or methyl;
   wherein m is 0 or 1; and
   Z¹² is —OR³⁰, —NR³¹R³², —C(O)NR³¹R³² or —NR³³C(O)R³⁴;
   wherein R³⁰ is (1-3C)alkyl, (4-6C)cycloalkyl, a carbon-linked 4 to 6-membered heterocyclyl, a 5 or 6 membered heteroaryl, —(CHRⱼ)ₒ-(4-6C)cycloalkyl, —(CHRⱼ)ₒ-[4 to 6-membered heterocyclyl] or —(CHRⱼ)ₒ-[5 or 6 membered heteroaryl], wherein R; is hydrogen or methyl and o is 0 or 1;
   R³¹ and R³² are each independently selected from hydrogen, (1-4C)alkyl, (2-4C)alkanoyl, (4-6C)cycloalkyl, a carbon-linked 4 to 6-membered heterocyclyl, a 5 or 6 membered heteroaryl, —(CHRᵢ)ₚ-(4-6C)cycloalkyl, —(CHRᵢ)ₚ-[4 to 6-membered heterocyclyl] or —(CHRᵢ)ₚ-[5 or 6 membered heteroaryl], wherein Rᵢ is hydrogen or methyl and p is 0 or 1
   or R³¹ and R³² are linked, such that, together with the nitrogen atom to which they are attached, they form a 4-6 membered heterocyclic ring;
   R³³ is hydrogen or (1-2C)alkyl;
   R³⁴ is (1-4C)alkyl, (2-4C)alkynyl, (4-6C)cycloalkyl, a carbon-linked 4 to 6-membered heterocyclyl, a 5 or 6 membered heteroaryl, —(CHRₕ)_{q}-(4-60)cycloalkyl, —(CHRₕ)_{q}-[4 to 6-membered heterocyclyl] or —(CHRₕ)_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is optionally substituted with one or more R^{a}.
(97) R¹⁴ is selected from halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
   wherein Rₖ is hydrogen or methyl;
   wherein m is 0 or 1; and
   Z¹² is —OR³⁰, —NR³¹R³², —C(O)NR³¹R³² or —NR³³C(O)R³⁴;
   wherein R³⁰ is (1-2C)alkyl, (5-6C)cycloalkyl, a carbon-linked 5 to 6-membered heterocyclyl, a 5 or 6 membered heteroaryl, —(CHRⱼ)ₒ-(5-6C)cycloalkyl, —(CHRⱼ)ₒ-[5 to 6-membered heterocyclyl] or —(CHRⱼ)ₒ-[5 or 6 membered heteroaryl], wherein R; is hydrogen or methyl and o is 0 or 1;
   R³¹ and R³² are each independently selected from hydrogen, (1-2C)alkyl, (2-3C)alkanoyl, (5-6C)cycloalkyl, a carbon-linked 5 to 6-membered heterocyclyl, a 5 or 6 membered heteroaryl, —(CHRᵢ)ₚ-(5-6C)cycloalkyl, —(CHRᵢ)ₚ-[5 to 6-membered heterocyclyl] or —(CHRᵢ)ₚ-[5 or 6 membered heteroaryl], wherein Rᵢ is hydrogen or methyl and p is 0 or 1
   or R³¹ and R³² are linked, such that, together with the nitrogen atom to which they are attached, they form a 5-6 membered heterocyclic ring;
   R³³ is hydrogen or (1-2C)alkyl;
   R³⁴ is (1-2C)alkyl, (2-3C)alkynyl, (5-6C)cycloalkyl, a carbon-linked 5 to 6-membered heterocyclyl, a 5 or 6 membered heteroaryl, —(CHRₕ)_{q}-(5-6C)cycloalkyl, —(CHRₕ)_{q}-[5 to 6-membered heterocyclyl] or —(CHRₕ)_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is optionally substituted with one or more R^{a}.
(98) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
   wherein Rₖ is hydrogen;
   wherein m is 0 or 1; and
   Z¹² is —OR³⁰, —NR³¹R³², —C(O)NR³¹R³² or —NR³³C(O)R³⁴;
   wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[⁴ to 8-membered heterocyclyl], wherein R; is hydrogen or methyl and o is 0 or 1;
   R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1
   or R³¹ and R³² are linked, such that, together with the nitrogen atom to which they are attached, they form a 4-8 membered heterocyclic ring;
   R³³ is hydrogen or methyl;
   R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[⁴ to 8-membered heterocyclyl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a}.
(99) Each R^{a} is independently selected from the group consisting of oxo, halogen, cyano, hydroxy, (1-4C)alkyl, or a group

-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —SO₂N(R^{14a})—, or —N(R^{14a})SO₂—, where R^{14a} is hydrogen or (1-2C)alkyl;
Q¹ is selected from the group consisting of hydrogen, (1-4C)alkyl, or (3-6C)cycloalkyl.
(100) Each R^{a} is independently selected from the group consisting of oxo, halogen, cyano, hydroxy, (1-2C)alkyl, or a group

-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —SO₂N(R^{14a})—, or —N(R^{14a})SO₂—, where R^{14a} is hydrogen or (1-2C)alkyl;
Q¹ is selected from the group consisting of hydrogen, (1-2C)alkyl, or (3-6C)cycloalkyl.
(101) Each R^{a} is independently selected from the group consisting of oxo, halogen, cyano, hydroxy, (1-2C)alkyl, or a group

-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, N(R^{14a})—C(O)—, or —NR^{14a}—, where R^{14a} is hydrogen or (1-2C)alkyl;
Q¹ is selected from the group consisting of hydrogen or (1-2C)alkyl.
(102) Each R^{a} is independently selected from the group consisting of oxo, halogen, cyano, hydroxy, (1-2C)alkyl, or a group

-L¹-X¹-Q¹
wherein:
L¹ is absent;
X¹ is absent or is selected from the group consisting of —O—, —C(O)—, —S(O)₀₋₂—, —C(O)—N(R^{14a})—, N(R^{14a})—C(O)—, or —NR^{14a}—, where R^{14a} is hydrogen or (1-2C)alkyl;
Q¹ is selected from the group consisting of hydrogen or (1-2C)alkyl.

Particular compounds of the invention also include, for example, compounds of the Formula (II), or pharmaceutically acceptable salts, hydrates and/or solvates thereof, wherein, unless otherwise stated, each of R², R⁴ (and R^{5a} and R^{5b}), R⁶ (and R⁷, n, R⁸, m, and R⁹), R¹¹ (and R^{11B}, R^{11C} and R^{11d}), R^{11A} Y₁, Y₂, A₂ (and R¹²), A₃ (and R¹³), A₄ (and R¹⁴) and R^{a}, and any associated substituent groups, has any of the meanings defined hereinbefore, in particular in any of paragraphs (1) to (102) above.

In particular, in compounds of formula (II):
R² is as defined in paragraph (1) or (2) above;
R⁴ is as defined in any one of paragraphs (3) to (13) above;
R^{5a} is as defined in any one of paragraphs (14) to (16) above;
R^{5b} is as defined in any one of paragraphs (17) to (20) above;
R⁶ is as defined in any one of paragraphs (21) to (26) above;
R⁷ is as defined in any one of paragraphs (27) to (30) above;
n is as defined in any one of paragraphs (31) to (33) above;
R⁸ is as defined in any one of paragraphs (34) to (44) above;
m is as defined in any one of paragraphs (45) to (47) above;
R⁹ is as defined in any one of paragraphs (48) to (51) above;
Y₁ is as defined in paragraph (52), (53) or (57) above;
Y₂ is as defined in paragraph (54), (55), (56) or (57) above;
R¹¹ is as defined in any one of paragraphs (62) to (64) above;
R^{11B} and R^{11C} are as defined in any one of paragraphs (65) to (69) above;
R^{11D} is as defined in any one of paragraphs (70) to (73) above;
R^{11E} is as defined in any one of paragraphs (74) to (80) above;
A₂, A₃ and A₄ are as defined in any one of paragraphs (81) to (88) above;
R¹² is as defined in any one of paragraphs (89) to (92) above;
R¹³ is as defined in any one of paragraphs (93) to (95) above;
R¹⁴ is as defined in any one of paragraphs (96) to (98) above; and
R^{a} is as defined in any one of paragraphs (99) to (102) above.

In addition, in compounds of formula (II), the groups R⁶, R⁸, A₁, A₂, A₃ A₄, R¹⁰, and any associated substituent groups, may be as defined hereinbefore or are as defined in any of paragraphs (103) to (124) hereinafter:
(103) R⁶ is (1-4C)alkyl, (1-4C)haloalkyl, (3-6C)cycloalkyl, or a 4- to 7-membered heterocyclyl ring comprising one heteroatom selected from N, O or S, or a group having a structure according to formula (A) shown below:

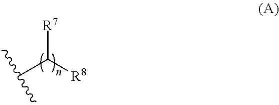

wherein n, R⁷ and R⁸ are each as defined hereinbefore, in particular n is as defined in paragraph (31) or (32) above, R⁷ is as defined in paragraph (27), (28), (29) or (30) above, and R⁸ is as defined in paragraph (33), (34), (35), (36), (37), (38), (39), (40), (41), (42), (43) or (44) above, or (107) below.
(104) R⁶ is (1-4C)alkyl, (1-4C)haloalkyl or a 5- or 6-membered heterocyclyl ring comprising one heteroatom selected from O or S; or a group having a structure according to formula (A) shown below:

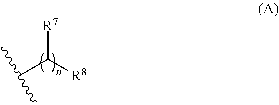

wherein n, R⁷ and R⁸ are each as defined hereinbefore, in particular n is as defined in paragraph (31) or (32) above, R⁷ is as defined in paragraph (27), (28), (29) or (30) above, and R⁸ is as defined in paragraph (33), (34), (35), (36), (37), (38), (39), (40), (41), (42), (43) or (44) above, or (107) below.
(105) R⁶ is (1-3C)alkyl, (1-3C)haloalkyl, tetrahydrofuran, tetrahydropyran or a group having a structure according to formula (A) shown below:

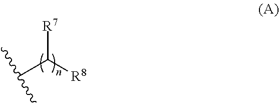

wherein n, R⁷ and R⁸ are each as defined hereinbefore, in particular n is as defined in paragraph (31) or (32) above, R⁷ is as defined in paragraph (27), (28), (29) or (30) above, and R⁸ is as defined in paragraph (33), (34), (35), (36), (37), (38), (39), (40), (41), (42), (43) or (44) above, or (107) below.
(106) R⁶ is methyl, ethyl, trifluoromethyl, trifluoroethyl or a group having a structure according to formula (A) shown below:

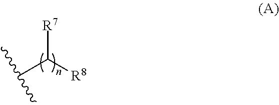

wherein n, R⁷ and R⁸ are each as defined hereinbefore, in particular n is as defined in paragraph (31) or (32) above, R⁷ is as defined in paragraph (27), (28), (29) or (30) above, and R⁸ is as defined in paragraph (33), (34), (35), (36), (37), (38), (39), (40), (41), (42), (43) or (44) above, or (107) below.
(107) R⁸ has any one of the following structures:

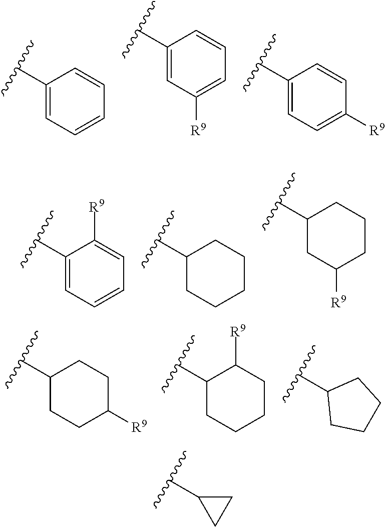

wherein R⁹ is as defined hereinbefore, in particular R⁹ is as defined in paragraph (48), (49), (50) or (51) above.
(108) A₁, is selected from N, CH or CR¹⁰;
   A₂ is selected from N, CH or CR¹⁰;
   A₃ is selected from N or CH;
   A₄ is selected from N, CH or CR¹⁴;
   with the proviso that:
   only one or two of A₁, A₂, A₃ or A₄ can be N; and
   only one of A₁ and A₂ can be CR¹⁰;
   wherein R¹⁰ is as defined hereinbefore, in particular R¹⁰ is as defined in paragraph (114) or (115) below;
   wherein R¹⁴ is as defined hereinbefore, in particular R¹⁴ is as defined in paragraph (96), (97), or (98) above;
(109) A₁ is selected from N, CH or CR¹⁰;
   A₂ is selected from N, CH or CR¹⁰;
   A₃ is selected from N, CH or CR¹³;
   A₄ is selected from N or CH;
   with the proviso that:
   only one or two of A₁, A₂, A₃ or A₄ can be N; and
   only one of A₁ and A₂ can be CR¹⁰;
   wherein R¹⁰ is as defined hereinbefore, in particular R¹⁰ is as defined in paragraph (114) or (115) below;
   wherein R¹³ is as defined hereinbefore, in particular R¹³ is as defined in paragraph (93), (94) or (95) above;
(110) A₁ is selected from N, CH or CR¹⁰;
   A₂ is selected from N, CH or CR¹⁰;
   A₃ is selected from N or CH;
   A₄ is selected from N or CH;
   with the proviso that:
   only one or two of A₁, A₂, A₃ or A₄ can be N; and
   only one of A₁ and A₂ can be CR¹⁰;
   wherein R¹⁰ is as defined hereinbefore, in particular R¹⁰ is as defined in paragraph (114) or (115) below;
(111) A₁, is selected from CH or CR¹⁰;
   A₂ is selected from CH or CR¹⁰;
   A₃ is CH;
   A₄ is CH;
   with the proviso that:
   only one of A₁, and A₂ can be CR¹⁰;
   wherein R¹⁰ is as defined hereinbefore, in particular R¹⁰ is as defined in paragraph (114) or (115) below;
(112) A₁, is CR¹⁰;
   A₂ is CH;
   A₃ is CH;
   A₄ is CH;
   wherein R¹⁰ is as defined hereinbefore, in particular R¹⁰ is as defined in paragraph (114) or (115) below;
(113) A₁, is CH;
   A₂ is CR¹⁰;
   A₃ is CH;
   A₄ is CH;
   wherein R¹⁰ is as defined hereinbefore, in particular R¹⁰ is as defined in paragraph (114) or (115) below;
(114) R¹⁰ is a group of the formula (IIA) or (IIB) shown below:

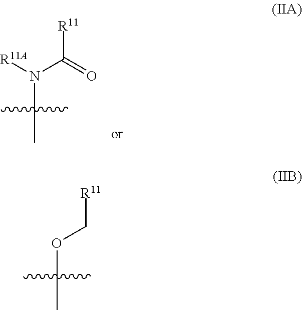

wherein [symbol] denotes the point of attachment to A₁, or A₂, and wherein R¹¹ and R^{11A} are as defined hereinbefore (e.g. in paragraphs (59) to (64) above).
(115) R¹⁰ is one of the groups shown below:

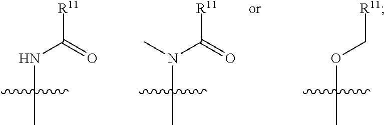

wherein [symbol] denotes the point of attachment to A₁ or A₂, and wherein R¹¹ is as defined hereinbefore (e.g. in paragraphs (62) to (64) above).
(116) R^{11B} and R^{11C} are each independently selected from the group consisting of hydrogen, halo, (1-4C)alkyl, and —C(O)NR^{11F}R^{11G}, wherein R^{11F} and R^{11G} are each independently selected from hydrogen or (1-4C)alkyl;
   and wherein any (1-4C)alkyl group present in a R^{11B} and/or R^{11C} group is optionally substituted with one or more substituents selected from halo, hydroxy, cyano, [(1-4C)alkyl]amino, di-[(1-4C)alkyl]amino, (1-4C)alkoxy, or a 4, 5 or 6-membered N-linked heterocycle which is optionally further substituted by one or more substituents selected from halo, hydroxy, cyano, (1-4C)alkyl, or (1-4C)alkoxy;
(117) R^{11B} and R^{11C} are each independently selected from the group consisting of hydrogen, halo, (1-2C)alkyl, and —C(O)NR^{11F}R^{11G}, wherein R^{11F} and R^{11G} are each independently selected from hydrogen or (1-2C)alkyl;
   and wherein any (1-2C)alkyl group present in a R^{11B} and/or R^{11C} group is optionally substituted with one or more substituents selected from halo, hydroxy, cyano, [(1-2C)alkyl]amino, di-[(1-2C)alkyl]amino, (1-2C)alkoxy, or a 4, 5 or 6-membered N-linked heterocycle which is optionally further substituted by one or more substituents selected from halo, hydroxy, cyano, (1-4C)alkyl, or (1-4C)alkoxy;
(118) One of R^{11B} and R^{11C} is hydrogen and the other is selected from the group consisting of hydrogen, halo, (1-2C)alkyl and —C(O)NR^{11F}R^{11G}, wherein Ru^{11F} and R^{11G} are each independently selected from hydrogen or (1-2C)alkyl;
   and wherein any (1-2C)alkyl group present in a R^{11B} and/or R^{11C} group is optionally substituted with one or more substituents selected from halo, hydroxy, cyano, [(1-2C)alkyl]amino, di-[(1-2C)alkyl]amino, (1-2C)alkoxy, or a 4, 5 or 6-membered N-linked heterocycle which is optionally further substituted by one or more substituents selected from halo, hydroxy, cyano, (1-2C)alkyl, or (1-2C)alkoxy;
(119) One of R^{11B} and R^{11C} is hydrogen and the other is selected from the group consisting of hydrogen, fluoro, (1-2C)alkyl and —C(O)NRF^{11F}R^{11G}, wherein R^{11F} and R^{11G} are each independently selected from hydrogen or (1-2C)alkyl;
   and wherein any (1-2C)alkyl group present in a R^{11B} and/or R^{11C} group is optionally substituted with one or more substituents selected from halo, [(1-2C)alkyl]amino, di-[(1-2C)alkyl]amino, (1-2C)alkoxy or a 4, 5 or 6-membered N-linked heterocycle which is optionally further substituted by one or more substituents selected from halo, hydroxy, cyano, (1-4C)alkyl, or (1-4C)alkoxy;
(120) One of R^{11B} and R^{11C} is hydrogen and the other is selected from the group consisting of hydrogen, fluoro, (1-2C)alkyl and —C(O)NR^{11F}R^{11G}, wherein R^{11F} and R^{11G} are each independently selected from hydrogen or (1-2C)alkyl;
   and wherein any (1-2C)alkyl group present in a R^{11B} and/or Ru G group is optionally substituted with one or more substituents selected from chloro, fluoro, [(1-2C)alkyl]amino, di-[(1-2C)alkyl]amino, (1-2C)alkoxy or a 5 or 6-membered N-linked heterocycle which is optionally further substituted by one or more substituents selected from chloro, fluoro, hydroxy, cyano, (1-2C)alkyl, or (1-2C)alkoxy;
(121) R^{11D} is selected from hydrogen, halo, (1-2C)alkyl and —C(O)H, wherein any (1-2C)alkyl or —C(O)H group is optionally substituted with one or more substituents selected from halo, hydroxy, cyano, [(1-2C)alkyl]amino, di-[(1-2C)alkyl]amino, (1-2C)alkoxy, (4-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-2C)alkyl, (1-2C)alkoxy, (4-6C)cycloalkyl, (4-6C)cycloalkoxy or (1-2C)alkyl;
(122) R^{11D} is selected from hydrogen, halo, (1-2C)alkyl and —C(O)H, wherein any (1-2C)alkyl or —C(O)H group is optionally substituted with one or more substituents selected from halo, hydroxy, [(1-2C)alkyl]amino, di-[(1-2C)alkyl]amino or 4- to 6-membered heterocyclyl;
(123) R^{11D} is selected from hydrogen, chloro, fluoro, methyl and ethyl, wherein any methyl or ethyl is optionally substituted with one or more substituents selected from chloro, fluoro, [(1-2C)alkyl]amino, di-[(1-2C)alkyl]amino or a 5- or 6-membered heterocyclyl;
(124) R^{11D} is selected from hydrogen, fluoro or methyl, wherein any methyl is optionally substituted with one or more substituents selected from —NMe₂ or morpholine.

Suitably, R² is as defined in numbered paragraph (1). Most suitably, R² is as defined in numbered paragraph (2).

Suitably, R⁴ is as defined in any one of numbered paragraphs (5) to (13). More suitably R⁴ is as defined in any one of numbered paragraphs (9) to (13). Most suitably, R⁴ is as defined in any one of numbered paragraphs (11), (12) or (13).

Suitably, R^{5a} is as defined in anyone of numbered paragraphs (15) or (16). Most suitably, R^{5a} is as defined in numbered paragraph (16).

Suitably, R^{5b} is as defined in anyone of numbered paragraphs (19) or (20). Most suitably, R^{5b} is as defined in numbered paragraph (20).

Suitably, R⁶ is as defined in any one of numbered paragraphs to (22) to (26), or (103) to (106). More suitably, R⁵ is as defined in any one of numbered paragraphs to (24) to (26), or (104) to (106). Even more suitably, R⁶ is as defined in any one of numbered paragraphs (25), (26), (105) or (106). Most suitably, R⁶ is as defined in numbered paragraph to (26).

Suitably, R⁷ is as defined in any one of numbered paragraphs (28) to (30). Most suitably, R⁷ is as defined in any one of numbered paragraphs (29) or (30).

Suitably, n is as defined in any one of numbered paragraphs (31) or (32). Most suitably, n is as defined in numbered paragraph (31).

Suitably, R⁸ is as defined in any one of numbered paragraphs (37) to (44), or (107). More suitably, R⁸ is as defined in any one of numbered paragraphs to (40) to (44), or (107). Most suitably, R⁸ is as defined in any one of numbered paragraphs (42), (43), (44) or (107).

Suitably, m is as defined in any one of numbered paragraphs (46) or (47). Most suitably, m is as defined in numbered paragraph (47).

Suitably, R⁹ is as defined in any one of numbered paragraphs (50) or (51). Most suitably, R⁹ is as defined in numbered paragraph (51).

Suitably, Y₁ is as defined in any one of numbered paragraphs (52) or (53). Most suitably, Y₁, is as defined in numbered paragraph (53).

Suitably, Y₂ is as defined in any one of numbered paragraphs (55) to (57). Most suitably, Y₂ is as defined in any one of numbered paragraphs (56) or (57).

Suitably, Y₁ and Y₂ are as defined in numbered paragraph (58).

Suitably, R^{11A} is as defined in any one of numbered paragraphs (60) or (61). Most suitably, R^{11A} is as defined in numbered paragraph (61).

Suitably, R¹¹ is as defined in any one of numbered paragraphs (63) or (64).

Suitably, R^{11B} and R^{11C} are as defined in any one of numbered paragraphs (66) to (69), or (116) to (120). More suitably, R^{11B} and R^{11C} are as defined in any one of numbered paragraphs (67), (68) or (69), or (117) to (120). Even more suitably, R^{11B} and R^{11C} are as defined in any one of numbered paragraphs (68), (69), (118), (119) or (120). Most suitably, R^{11B} and R^{11C} are as defined in numbered paragraph (69).

Suitably, R^{11D} is as defined in any one of numbered paragraphs (71), (72) or (73), or (121) to (124). More suitably, R^{11D} is as defined in any one of numbered paragraphs (72), (73), (122), (123) or (124) Most suitably, R^{11D} is as defined in numbered paragraph (73).

Suitably, R^{11E} is as defined in any one of numbered paragraphs (77) to (80). Most suitably, R^{11E} is as defined in any one of numbered paragraphs (79) or (80).

Suitably, A₂, A₃ and A₄ are as defined in any one of numbered paragraphs (84) to (88). Most suitably, A₂, A₃ and A₄ are as defined in numbered paragraph (88).

Suitably, A₁, A₂, A₃ and A₄ are as defined in any one of numbered paragraphs (108) to (113). More suitably, A₁, A₂, A₃ and A₄ are as defined in any one of numbered paragraphs (109) to (113). Even more suitably, A₁, A₂, A₃ and A₄ are as defined in any one of numbered paragraphs (109) to (113). Yet even more suitably, A₁, A₂, A₃ and A₄ are as defined in any one of numbered paragraphs (110) to (113). Most suitably, A₂, A₃ and A₄ are as defined in any one of numbered paragraphs (111), (112) or (113).

Suitably, R¹² is as defined in any one of numbered paragraphs (90), (91), (92). Most suitably, R¹² is as defined in numbered paragraph (92).

Suitably, R¹⁰ is as defined in any one of numbered paragraphs (114) or (115). More suitably, R¹⁰ is as defined in numbered paragraphs (115).

Suitably, R¹³ is as defined in any one of numbered paragraphs (94) or (95). Most suitably, R¹³ is as defined in numbered paragraph (95).

Suitably, R¹⁴ is as defined in any one of numbered paragraphs (97) or (98). Most suitably, R¹⁴ is as defined in numbered paragraph (98).

Suitably, R^{a} is as defined in any one of numbered paragraphs (100), (101) or (102). Most suitably, R^{a} is as defined in numbered paragraph (102).

In a particular group of compounds of the invention, compounds have a structure according to formula I-I, I-II, I-III, I-IV, I-V or I-VI (which are sub-definitions of formula I), or a pharmaceutically acceptable salt, hydrate and/or solvate thereof:

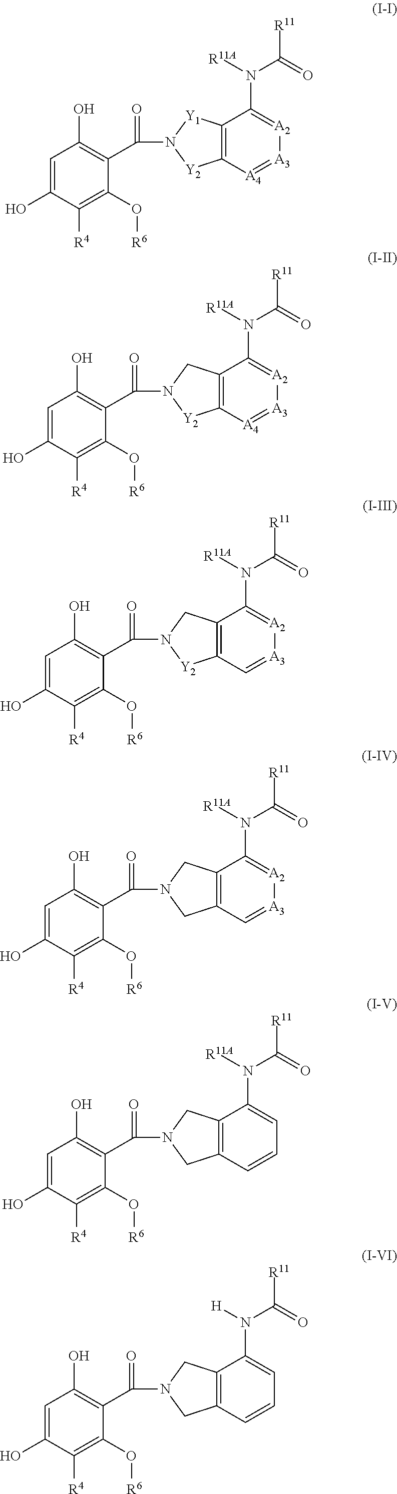

wherein
R⁴, R⁶, Y₁, Y₂, A₂, A₃, A₄, R¹¹, R^{11A} and any associated subgroups, are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
Y¹ is as defined in any one of numbered paragraphs (52) or (53);
Y² is as defined in any one of numbered paragraphs (55) to (57);
R^{11A} is as defined in any one of numbered paragraphs (59) to (61);
R¹¹ is as defined in any one of numbered paragraphs (63) or (64);
A₂, A₃ and A₄ are as defined in any one of numbered paragraphs (81) to (88); and all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ is as defined in any one of numbered paragraphs (52) or (53);
Y² is as defined in any one of numbered paragraphs (55) to (57);
R^{11A} is as defined in any one of numbered paragraphs (59) to (61);
R¹¹ is as defined in any one of numbered paragraphs (63) or (64);
R^{11B} and R^{11D} are as defined in any one of numbered paragraphs (66) to (69), or (116) to (120);
R^{11D} is as defined in any one of numbered paragraphs (71) to (73), or (121) to (124);
R^{11E} is as defined in any one of numbered paragraphs (77) to (80);
A₂, A₃ and A₄ are as defined in any one of numbered paragraphs (84) to (88); and all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in any one of numbered paragraphs (59) to (61);
R¹¹ is as defined in any one of numbered paragraphs (63) or (64);
R^{11B} and R^{11C} are as defined in any one of numbered paragraphs (66) to (69), or (116) to (120);
R^{11D} is as defined in any one of numbered paragraphs (71) to (73), or (121) to (124);
R^{11E} is as defined in any one of numbered paragraphs (77) to (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (106);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
R^{11A} is as defined in any one of numbered paragraphs (59) to (61);
R¹¹ is as defined in any one of numbered paragraphs (63) or (64);
R^{11B} and R^{11C} are as defined in any one of numbered paragraphs (66) to (69), or (116) to (120);
R^{11D} is as defined in any one of numbered paragraphs (71) to (73), or (121) to (124);
R^{11E} is as defined in any one of numbered paragraphs (77) to (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
R^{11A} is as defined in any one of numbered paragraphs (60) or (61);
R¹¹ is as defined in any one of numbered paragraphs (63) or (64);
R^{11B} and R^{11C} are as defined in any one of numbered paragraphs (67) to (69), or (117) to (120);
R^{11D} is as defined in any one of numbered paragraphs (72), (73), (122), (123) or (124);
R^{11E} is as defined in any one of numbered paragraphs (78) to (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
R^{11A} is as defined in numbered paragraph (61);
R¹¹ is as defined in any one of numbered paragraphs (63) or (64);
R^{11B} and R^{11C} are as defined in any one of numbered paragraphs (69), (119) or (120);
R¹¹ is as defined in any one of numbered paragraphs (73) or (124);
R^{11E} is as defined in any one of numbered paragraphs (79) or (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in numbered paragraph (61);
R¹¹ is as defined in any one of numbered paragraphs (63) or (64);
R^{11B} and R^{11C} are as defined in any one of numbered paragraphs (67) to (69), or (116) to (120);
R^{11D} is as defined in any one of numbered paragraphs (73) or (124);
R^{11E} is as defined in any one of numbered paragraphs (78) to (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in numbered paragraph (61);
R¹¹ is as defined in any one of numbered paragraphs (63) or (64);
R^{11B} and R^{11C} are as defined in any one of numbered paragraphs (67) to (69), or (116) to (120);
R^{11D} is as defined in any one of numbered paragraphs (73) or (124);
R^{11E} is as defined in any one of numbered paragraphs (78) to (80);
A₂, A₃ and A₄ are as defined in any one of numbered paragraphs (84) to (88);
R¹² is as defined in any one of numbered paragraphs (90) to (92);
R¹³ is as defined in any one of numbered paragraphs (94) or (95);
R¹⁴ is as defined in any one of numbered paragraphs (97) or (98);
R^{a} is as defined in any one of numbered paragraphs (100) to (102); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (106);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
R^{11A} is as defined in numbered paragraph (61);
R¹¹ is as defined in any one of numbered paragraphs (63) or (64);
R^{11B} and R^{11C} are as defined in any one of numbered paragraphs (69) or (120);
R^{11D} is as defined in any one of numbered paragraphs (73) or (124);
R^{11E} is as defined in any one of numbered paragraphs (79) or (80);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in numbered paragraph (12);
R⁶ is as defined in numbered paragraph (26) or (106);
R⁷ is as defined numbered paragraph (29);
n is as defined in numbered paragraph (31);
R⁸ is as defined in numbered paragraphs (44), or (107);
Y¹ and Y² are as defined in numbered paragraph (58);
R^{11A} is as defined in numbered paragraph (61);
R¹¹ is as defined in numbered paragraph (63);
R^{11B} and R^{11D} are as defined in paragraph (69) or (120);
R^{11D} is as defined in paragraph (73) or (124);
R^{11E} is as defined in numbered paragraphs (79);
A₂, A₃ and A₄ are as defined in numbered paragraph (88); and
all other groups are as defined in any of the numbered paragraphs appearing hereinbefore.

In an embodiment of the compounds of formula I-I, I-II, I-III, I-IV, I-V or I-VI, or a pharmaceutically acceptable salt, hydrate and/or solvate thereof, where appropriate:
R⁴ is as defined in numbered paragraph (13);
R⁶ is as defined in numbered paragraph (106);
R⁷ is as defined numbered paragraph (30);
n is as defined in numbered paragraph (31);
R⁸ is as defined in numbered paragraphs (107);
Y¹ and Y² are as defined in numbered paragraph (58);
R^{11A} is as defined in numbered paragraph (61);
R¹¹ is as defined in numbered paragraph (64);
R^{11B} and R^{11D} are as defined in paragraph (120);
R^{11D} is as defined in paragraph (124);
R^{11E} is as defined in numbered paragraphs (80);
A₂, A₃ and A₄ are as defined in numbered paragraph (88); and
all other groups are as defined in any of the numbered paragraphs appearing hereinbefore.

In a particular group of compounds of the invention, compounds have a structure according to formula I-VII, I-VIII, I-IX, I-X, I-XI or I-XII (which are sub-definitions of formula I), or a pharmaceutically acceptable salt, hydrate and/or solvate thereof:

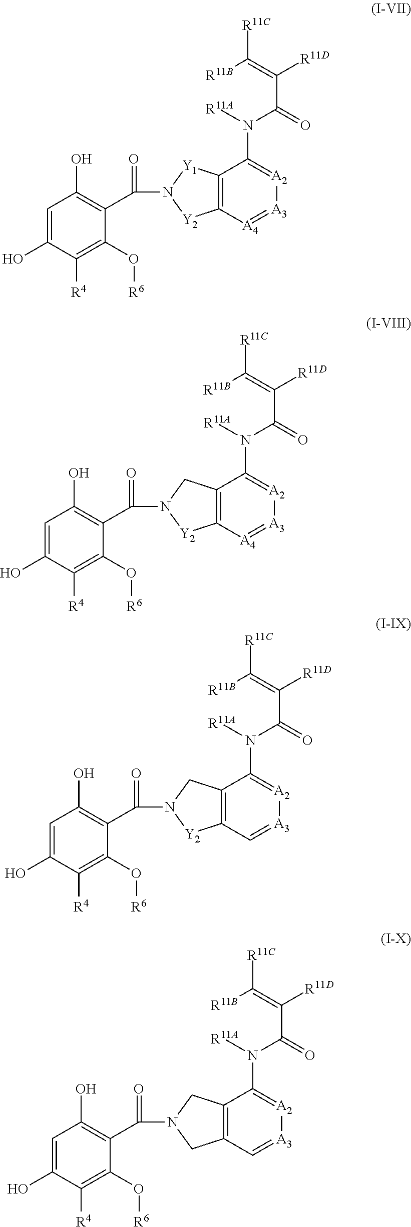

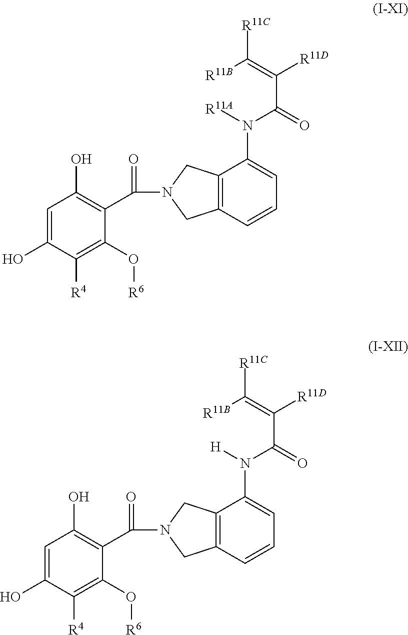

wherein
R⁴, R⁶, Y₁, Y₂, A₂, A₃, A₄, R^{11A}, R^{11B}, R^{11C}, R^{11D} and any associated subgroups, are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
Y¹ is as defined in any one of numbered paragraphs (52) or (53);
Y² is as defined in any one of numbered paragraphs (55) to (57);
R^{11A} is as defined in any one of numbered paragraphs (59) to (61);
R^{11B} and R^{11D} are as defined in any one of numbered paragraphs (66) to (69), or (116) to (120);
R^{11D} is as defined in any one of numbered paragraphs (71) to (73), or (121) to (124);
A₂, A₃ and A₄ are as defined in any one of numbered paragraphs (81) to (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ is as defined in any one of numbered paragraphs (52) or (53);
Y² is as defined in any one of numbered paragraphs (55) to (57);
R^{11A} is as defined in any one of numbered paragraphs (59) to (61);
R^{11B} and R^{11D} are as defined in any one of numbered paragraphs (66) to (69), or (116) to (120);
R^{11D} is as defined in any one of numbered paragraphs (71) to (73), or (121) to (124);
A₂, A₃ and A₄ are as defined in any one of numbered paragraphs (84) to (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in any one of numbered paragraphs (59) to (61);
R^{11B} and R^{11C} are as defined in any one of numbered paragraphs (66) to (69), or (116) to (120);
R^{11D} is as defined in any one of numbered paragraphs (71) to (73), or (121) to (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (106);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
R^{11A} is as defined in any one of numbered paragraphs (59) to (61);
R^{11B} and R^{11C} are as defined in any one of numbered paragraphs (66) to (69), or (116) to (120);
R^{11D} is as defined in any one of numbered paragraphs (71) to (73), or (121) to (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
R^{11A} is as defined in any one of numbered paragraphs (60) or (61);
R^{11B} and R^{11C} are as defined in any one of numbered paragraphs (67) to (69), or (116) to (120);
R^{11D} is as defined in any one of numbered paragraphs (72), (73), (122), (123) or (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
R^{11A} is as defined in numbered paragraph (61);
R^{11B} and R^{11D} are as defined in any one of numbered paragraphs (69) or (120);
R^{11D} is as defined in any one of numbered paragraphs (73) or (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in numbered paragraph (61);
R^{11B} and R^{11D} are as defined in any one of numbered paragraphs (67) to (69), or (116) to (120);
R^{11D} is as defined in any one of numbered paragraphs (73) or (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
R^{11A} is as defined in numbered paragraph (61);
R^{11B} and R^{11D} are as defined in any one of numbered paragraphs (67) to (69), or (116) to (120);
R^{11D} is as defined in any one of numbered paragraphs (73) or (124);
A₂, A₃ and A₄ are as defined in any one of numbered paragraphs (84) to (88);
R¹² is as defined in any one of numbered paragraphs (90) to (92), (114) or (115);
R¹³ is as defined in any one of numbered paragraphs (94) or (95);
R¹⁴ is as defined in any one of numbered paragraphs (97) or (98);
R^{a} is as defined in any one of numbered paragraphs (100) to (102); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (106);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
R^{11A} is as defined in numbered paragraph (61);
R^{11B} and R^{11C} are as defined in any one of numbered paragraphs (69) or (120);
R^{11D} is as defined in any one of numbered paragraphs (73) or (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in numbered paragraph (12);
R⁶ is as defined in numbered paragraph (26) or (106);
R⁷ is as defined in numbered paragraph (29);
n is as defined in numbered paragraph (31);
R⁸ is as defined in numbered paragraph (44), or (107);
Y¹ and Y² are as defined in numbered paragraph (58);
R^{11A} is as defined in numbered paragraph (61);
R^{11B} and R^{11C} are as defined in numbered paragraph (69) or (120);
R^{11D} is as defined in numbered paragraph (73) or (124);
A₂, A₃ and A₄ are as defined in numbered paragraph (88); and
all other groups are as defined in any of the numbered paragraphs appearing hereinbefore.

In an embodiment of the compounds of formula I-VII, I-VIII, I-IX, I-X, I-XI or I-XII, or a pharmaceutically acceptable salt, hydrate and/or solvate thereof, where appropriate:
R⁴ is as defined in numbered paragraph (13);
R⁶ is as defined in numbered paragraph (106);
R⁷ is as defined in numbered paragraph (29);
n is as defined in numbered paragraph (31);
R⁸ is as defined in numbered paragraph (107);
Y¹ and Y² are as defined in numbered paragraph (58);
R^{11A} is as defined in numbered paragraph (61);
R^{11B} and R^{11D} are as defined in numbered paragraph (120);
R^{11D} is as defined in numbered paragraph (124);
A₂, A₃ and A₄ are as defined in numbered paragraph (88); and
all other groups are as defined in any of the numbered paragraphs appearing hereinbefore.

In a particular group of compounds of the invention, compounds have a structure according to formula —XIII, I-XIV, I-XV, I-XVI, I-XVII or I-XVIII (which are sub-definitions of formula I), or a pharmaceutically acceptable salt, hydrate and/or solvate thereof:

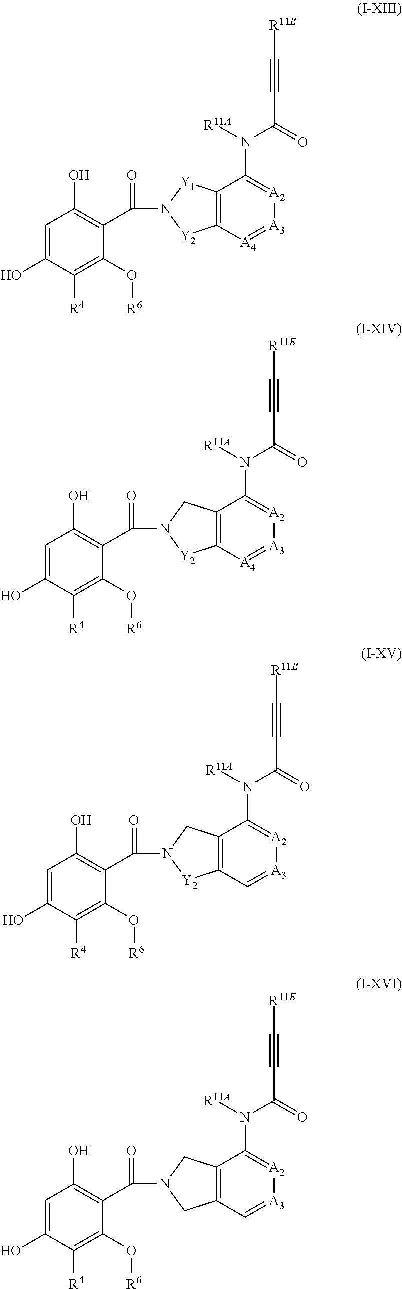

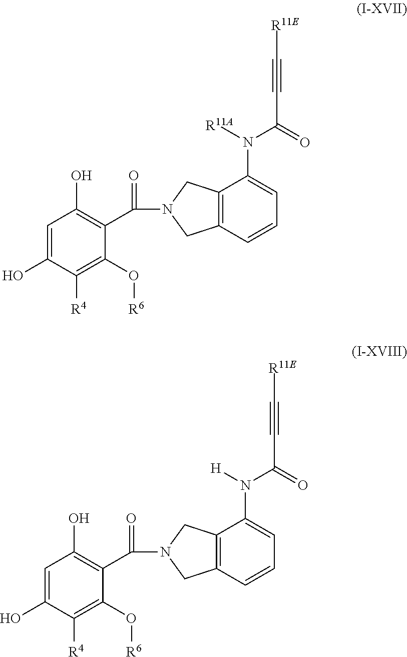

wherein
R⁴, R⁶, Y₁, Y₂, A₂, A₃, A₄, R^{11A}, R^{11E} and any associated subgroups, are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26);
Y¹ is as defined in any one of numbered paragraphs (52) or (53);
Y² is as defined in any one of numbered paragraphs (55) to (57);
R^{11A} is as defined in any one of numbered paragraphs (59) to (61);
R^{11E} is as defined in any one of numbered paragraphs (77) to (80);
A₂, A₃ and A₄ are as defined in any one of numbered paragraphs (81) to (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26);
R⁷ is as defined in any one of numbered paragraphs (28) to (30);
n is as defined in any one of numbered paragraphs (31) or (32);
R⁸ is as defined in any one of numbered paragraphs (37) to (44);
Y¹ is as defined in any one of numbered paragraphs (52) or (53);
Y² is as defined in any one of numbered paragraphs (55) to (57);
R^{11A} is as defined in any one of numbered paragraphs (59) to (61);
R^{11E} is as defined in any one of numbered paragraphs (77) to (80);
A₂, A₃ and A₄ are as defined in any one of numbered paragraphs (84) to (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26);
R⁷ is as defined in any one of numbered paragraphs (29) or (30);
n is as defined in numbered paragraph (31);
R⁸ is as defined in any one of numbered paragraphs (40) to (44);
Y¹ and Y² are as defined in numbered paragraph (58);
R^{11A} is as defined in any one of numbered paragraphs (59) to (61);
R^{11E} is as defined in any one of numbered paragraphs (77) to (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (11) to (13);
R⁶ is as defined in numbered paragraph (26);
R⁷ is as defined in any one of numbered paragraphs (29) or (30);
n is as defined in numbered paragraph (31);
R⁸ is as defined in any one of numbered paragraphs (42) to (44);
Y¹ and Y² are as defined in numbered paragraph (58);
R^{11A} is as defined in any one of numbered paragraphs (59) to (61);
R^{11E} is as defined in any one of numbered paragraphs (77) to (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26);
R⁷ is as defined in any one of numbered paragraphs (28) to (30);
n is as defined in any one of numbered paragraphs (31) or (32);
R⁸ is as defined in any one of numbered paragraphs (37) to (44);
Y¹ and Y² are as defined in numbered paragraph (58);
R^{11A} is as defined in any one of numbered paragraphs (60) or (61);
R^{11E} is as defined in any one of numbered paragraphs (78) to (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26);
R⁷ is as defined in any one of numbered paragraphs (28) to (30);
n is as defined in any one of numbered paragraphs (31) or (32);
R⁸ is as defined in any one of numbered paragraphs (37) to (44);
Y¹ and Y² are as defined in numbered paragraph (58);
R^{11A} is as defined in numbered paragraph (61);
R^{11E} is as defined in any one of numbered paragraphs (79) or (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26);
R⁷ is as defined in any one of numbered paragraphs (29) or (30);
n is as defined in numbered paragraph (31);
R⁸ is as defined in any one of numbered paragraphs (40) to (44);
Y¹ and Y² are as defined in numbered paragraph (58);
R^{11A} is as defined in numbered paragraph (61);
R^{11E} is as defined in any one of numbered paragraphs (78) to (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26);
R⁷ is as defined in any one of numbered paragraphs (29) or (30);
n is as defined in numbered paragraph (31);
R⁸ is as defined in any one of numbered paragraphs (40) to (44);
Y¹ and Y² are as defined in numbered paragraph (58);
R^{11A} is as defined in numbered paragraph (61);
R^{11E} is as defined in any one of numbered paragraphs (78) to (80);
A₂, A₃ and A₄ are as defined in any one of numbered paragraphs (84) to (88);
R¹² is as defined in any one of numbered paragraphs (90) to (92);
R¹³ is as defined in any one of numbered paragraphs (94) or (95);
R¹⁴ is as defined in any one of numbered paragraphs (97) or (98);
R^{a} is as defined in any one of numbered paragraphs (100) to (102); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in any one of numbered paragraphs (11) to (13);
R⁶ is as defined in numbered paragraph (26);
R⁷ is as defined in any one of numbered paragraphs (29) or (30);
n is as defined in numbered paragraph (31);
R⁸ is as defined in any one of numbered paragraphs (42) to (44);
Y¹ and Y² are as defined in numbered paragraph (58);
R^{11A} is as defined in numbered paragraph (61);
R^{11E} is as defined in any one of numbered paragraphs (79) or (80);
A₂, A₃ and A₄ are as defined in numbered paragraph (88); and
all other groups are as defined in any of the numbered paragraphs appearing hereinbefore.

In an embodiment of the compounds of formula I-XIII, I-XIV, I-XV, I-XVI, I-XVII or I-XVIII, or a pharmaceutically acceptable salt, hydrate and/or solvate thereof, where appropriate:
R⁴ is as defined in numbered paragraph (13);
R⁶ is as defined in numbered paragraph (26);
R⁷ is as defined in numbered paragraph (30);
n is as defined in numbered paragraph (31);
R⁸ is as defined in numbered paragraph (44);
Y¹ and Y² are as defined in numbered paragraph (58);
R^{11A} is as defined in numbered paragraph (61);
R^{11E} is as defined in numbered paragraph (80);
A₂, A₃ and A₄ are as defined in numbered paragraph (88); and
all other groups are as defined in any of the numbered paragraphs appearing hereinbefore.

In a particular group of compounds of the invention, compounds have a structure according to formula II-I, II-I, II-III, II-IV, II-V or II-VI (which are sub-definitions of formula II), or a pharmaceutically acceptable salt, hydrate and/or solvate thereof:

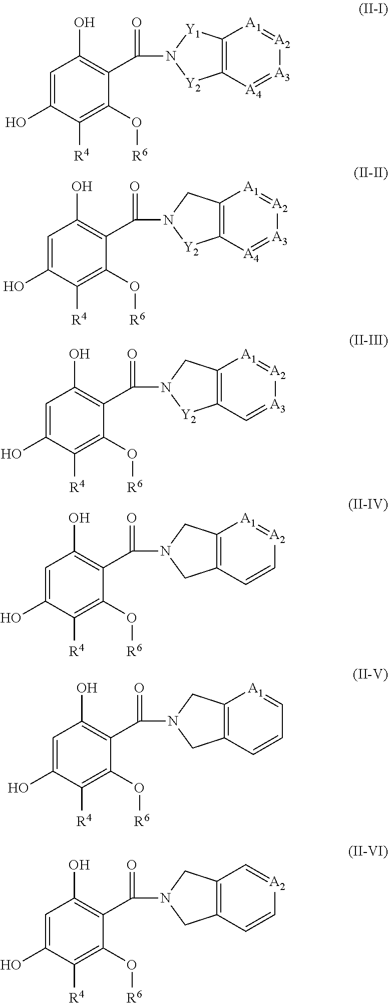

wherein
R⁴, R⁶, Y₁, Y₂, A₁, A₂, A₃ and A₄ and any associated subgroups, are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
Y¹ is as defined in any one of numbered paragraphs (52) or (53);
Y² is as defined in any one of numbered paragraphs (55) to (57);
A₁, A₂, A₃ and A₄ are as defined in any one of numbered paragraphs (108) to (113); and
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ is as defined in any one of numbered paragraphs (52) or (53);
Y² is as defined in any one of numbered paragraphs (55) to (57);
A₁, A₂, A₃ and A₄ are as defined in any one of numbered paragraphs (109) to (113); and
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
A₁, A₂, A₃ and A₄ are as defined in any one of numbered paragraphs (110) to (113);
R¹⁰ is as defined in any one of numbered paragraphs (114) or (115);
R^{11A} is as defined in any one of numbered paragraphs (59) to (61);
R¹¹ is as defined in any one of numbered paragraphs (63) or (64);
R^{11B} and R^{11C} are as defined in any one of numbered paragraphs (66) to (69), or (116) to (120);
R^{11D} is as defined in any one of numbered paragraphs (71) to (73), or (121) to (124);
R^{11E} is as defined in any one of numbered paragraphs (77) to (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (126);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
A₁, A₂, A₃ and A₄ are as defined in any one of numbered paragraphs (111) to (113);
R¹⁰ is as defined in any one of numbered paragraphs (114) or (115);
R^{11A} is as defined in any one of numbered paragraphs (60) or (61);
R¹¹ is as defined in any one of numbered paragraphs (63) or (64);
R^{11B} and R^{11C} are as defined in any one of numbered paragraphs (66) to (69), or (116) to (120);
R^{11D} is as defined in any one of numbered paragraphs (71) to (73), or (121) to (124);
R^{11E} is as defined in any one of numbered paragraphs (77) to (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
A₁, A₂, A₃ and A₄ are as defined in any one of numbered paragraphs (112) or (113);
R¹⁰ is as defined in any one of numbered paragraphs (114) or (115);
R^{11A} is as defined in any one of numbered paragraphs (60) or (61);
R¹¹ is as defined in any one of numbered paragraphs (63) or (64);
R^{11B} and R^{11C} are as defined in any one of numbered paragraphs (67) to (69), or (117) to (120);
R^{11D} is as defined in any one of numbered paragraphs (72), (73), (122), (123) or (124);
R^{11E} is as defined in any one of numbered paragraphs (78) to (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (5) to (13);
R⁶ is as defined in any one of numbered paragraphs (22) to (26), or (103) to (106);
R⁷ is as defined in any one of numbered paragraphs (28) to (30);
n is as defined in any one of numbered paragraphs (31) or (32);
R⁸ is as defined in any one of numbered paragraphs (37) to (44), or (107);
Y¹ and Y² are as defined in numbered paragraph (58);
A₁, A₂, A₃ and A₄ are as defined in any one of numbered paragraphs (112) or (113);
R¹⁰ is as defined in any one of numbered paragraphs (114) or (115);
R^{11A} is as defined in numbered paragraph (61);
R¹¹ is as defined in any one of numbered paragraphs (63) or (64);
R^{11B} and R^{11D} are as defined in any one of numbered paragraphs (69), (119) or (120);
R^{11D} is as defined in any one of numbered paragraphs (73) or (124);
R^{11E} is as defined in any one of numbered paragraphs (79) or (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (9) to (13);
R⁶ is as defined in any one of numbered paragraphs (24) to (26), or (104) to (106);
R⁷ is as defined in any one of numbered paragraphs (29) or (30);
n is as defined in numbered paragraph (31);
R⁸ is as defined in any one of numbered paragraphs (40) to (44), or (107);
Y¹ and Y² are as defined in numbered paragraph (58);
A₁, A₂, A₃ and A₄ are as defined in any one of numbered paragraphs (112) or (113);
R¹⁰ is as defined in any one of numbered paragraphs (114) or (115);
R^{11A} is as defined in numbered paragraph (61);
R¹¹ is as defined in any one of numbered paragraphs (63) or (64);
R^{11B} and R^{11C} are as defined in any one of numbered paragraphs (67) to (69), or (116) to (120);
R^{11D} is as defined in any one of numbered paragraphs (73) or (124);
R^{11E} is as defined in any one of numbered paragraphs (78) to (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in any one of numbered paragraphs (11) to (13);
R⁶ is as defined in any one of numbered paragraphs (26) or (126);
R⁷ is as defined in any one of numbered paragraphs (29) or (30);
n is as defined in numbered paragraph (31);
R⁸ is as defined in any one of numbered paragraphs (42) to (44), or (107);
Y¹ and Y² are as defined in numbered paragraph (58);
A₁, A₂, A₃ and A₄ are as defined in any one of numbered paragraphs (112) or (113);
R¹⁰ is as defined in any one of numbered paragraphs (114) or (115);
R^{11A} is as defined in numbered paragraph (61);
R¹¹ is as defined in any one of numbered paragraphs (63) or (64);
R^{11B} and R^{11C} are as defined in any one of numbered paragraphs (69) or (120);
R^{11D} is as defined in any one of numbered paragraphs (73) or (124);
R^{11E} is as defined in any one of numbered paragraphs (79) or (80);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in numbered paragraph (12);
R⁶ is as defined in numbered paragraph (26);
R⁷ is as defined in numbered paragraph (29);
n is as defined in numbered paragraph (31);
R⁸ is as defined in numbered paragraphs (44);
Y¹ and Y² are as defined in numbered paragraph (58);
A₁, A₂, A₃ and A₄ are as defined in numbered paragraph (112);
R¹⁰ is as defined in numbered paragraph (114);
R^{11A} is as defined in numbered paragraph (61);
R¹¹ is as defined in numbered paragraph (63);
R^{11B} and R^{11C} are as defined in numbered paragraph (69);
R^{11D} is as defined in numbered paragraph (73);
R^{11E} is as defined in numbered paragraph (79);
all other groups are as defined in any of the numbered paragraphs appearing hereinbefore.

In an embodiment of the compounds of formula II-I, II-II, II-III, II-IV, II-V or II-VI, or a pharmaceutically acceptable salt, hydrate and/or solvate thereof, where appropriate:
R⁴ is as defined in numbered paragraph (13);
R⁶ is as defined in numbered paragraph (106);
R⁷ is as defined in numbered paragraph (30);
n is as defined in numbered paragraph (31);
R⁸ is as defined in numbered paragraph (107);
Y¹ and Y² are as defined in numbered paragraph (58);
A₁, A₂, A₃ and A₄ are as defined in numbered paragraph (113);
R¹⁰ is as defined in numbered paragraph (115);
R^{11A} is as defined in numbered paragraph (61);
R¹¹ is as defined in numbered paragraph (64);
R^{11B} and R^{11C} are as defined in numbered paragraph (120);
R^{11D} is as defined in numbered paragraph (124);
R^{11E} is as defined in numbered paragraph (80);
all other groups are as defined in any of the numbered paragraphs appearing hereinbefore.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R² is as defined in paragraph (2) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁴ is as defined in paragraph (10) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁴ is as defined in paragraph (11) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁴ is as defined in paragraph (12) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁴ is as defined in paragraph (13) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (39) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (28) above, n is as defined in paragraph (31) above and R⁸ is as defined in paragraph (39) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (29) above, n is as defined in paragraph (31) above and R⁸ is as defined in paragraph (39) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (39) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (40) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (41) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (42) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (43) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R⁶ is as defined in paragraph (26) or (106) above, R⁷ is as defined in paragraph (27) above, n is as defined in paragraph (31) above and R⁸ is as defined in paragraph (44) or (107) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₁ is as defined in paragraph (52) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₁ is as defined in paragraph (53) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₂ is as defined in paragraph (55) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₂ is as defined in paragraph (56) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₂ is as defined in paragraph (57) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, Y₁ and Y₂ is as defined in paragraph (58) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R^{11A} is as defined in paragraph (59) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R^{11A} is as defined in paragraph (60) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R^{11A} is as defined in paragraph (61) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11B} and R^{11C} are as defined in paragraph (66) or (117) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11B} and R^{11C} are as defined in paragraph (67) or (118) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11B} and R^{11C} are as defined in paragraph (68) or (119) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11B} and R^{11C} are as defined in paragraph (69) or (120) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11D} is as defined in paragraph (70) or (121) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11D} is as defined in paragraph (71) or (122) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11D} is as defined in paragraph (72) or (123) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (63) above and R^{11D} is as defined in paragraph (73) or (124) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (74) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (75) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (76) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (77) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (78) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (79) above.

In an embodiment of the compounds of formula I or formula II or any appropriate sub-definition of formula I or formula II (as defined by formulae I-I to I-XVIII or formulae II-I to II-VI), or a pharmaceutically acceptable salt, hydrate and/or solvate thereof, R¹¹ is as defined in paragraph (64) above and R^{11E} is as defined in paragraph (80) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (81) above, R¹² is as defined in paragraph (89) above, R¹³ is as defined in paragraph (93) above and R¹⁴ is as defined in paragraph (96) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (82) above, R¹² is as defined in paragraph (89) above and R¹³ is as defined in paragraph (93) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (83) above and R¹³ is as defined in paragraph (93) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (84) above and R¹³ is as defined in paragraph (93) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (85) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (86) above and R¹² is as defined in paragraph (89) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (87) above and R¹⁴ is as defined in paragraph (96) above.

In an embodiment of the compounds of formula I or any appropriate sub-definition of formula I (as defined by formulae I-I to I-XVIII), or a pharmaceutically acceptable salt, hydrate and/or solvate thereof, A₂, A₃ and A₄ are as defined in paragraph (88) above.

In an embodiment of the compounds of formula I or any appropriate sub-definition of formula I (as defined by formulae I-I to I-XVIII), or a pharmaceutically acceptable salt, hydrate and/or solvate thereof, R^{a} is as defined in paragraph (99) above.

In an embodiment of the compounds of formula I or any appropriate sub-definition of formula I (as defined by formulae I-I to I-XVIII), or a pharmaceutically acceptable salt, hydrate and/or solvate thereof, R^{a} is as defined in paragraph (100) above.

In an embodiment of the compounds of formula I or any appropriate sub-definition of formula I (as defined by formulae I-I to I-XVIII), or a pharmaceutically acceptable salt, hydrate and/or solvate thereof, R^{a} is as defined in paragraph (101) above.

In an embodiment of the compounds of formula I or any appropriate sub-definition of formula I (as defined by formulae I-I to I-XVIII), or a pharmaceutically acceptable salt, hydrate and/or solvate thereof, R^{a} is as defined in paragraph (102) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (108) above, R¹⁰ is as defined in any one of paragraphs (114) or (115) above, R¹³ is as defined in paragraph (93) above and R¹⁴ is as defined in paragraph (96) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (109) above, R¹⁰ is as defined in any one of paragraphs (114) or (115) above and R¹³ is as defined in paragraph (93) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (110) above and R¹⁰ is as defined in any one of paragraphs (114) or (115) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (111) above, R¹⁰ is as defined in any one of paragraphs (114) or (115) above and R¹¹ is as defined in any one of paragraphs (63) or (64) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (112) or (113) above, R¹⁰ is as defined in any one of paragraphs (114) or (115) above, R¹¹ is as defined in any one of paragraphs (63) or (64) above and R^{11B} and R^{11C} are as defined in any one of paragraphs (67) to (69) or (118) to (120) above.

In an embodiment of the compounds of formula II or any appropriate sub-definition of formula II (as defined by formulae II-I to II-VI), or a pharmaceutically acceptable salt, hydrate and/or solvate thereof, A₁, A₂, A₃ and A₄ are as defined in paragraph (112) or (113) above, R¹⁰ is as defined in any one of paragraphs (114) or (115) above, R¹¹ is as defined in any one of paragraphs (63) or (64) above, R^{11B} and R^{11C} are as defined in any one of paragraphs (67) to (69) or (118) to (120) above, and RID is as defined in any one of paragraphs (71) to (73) or (122) to (124) above.

Particular compounds of the present invention include any of the compounds exemplified in the present application, or a pharmaceutically acceptable salt or solvate thereof, and, in particular, any of the following:
N-(2-(2-(Benzyloxy)-4,6-dihydroxybenzoyl)isoindolin-4-yl)acrylamide;
(E)-N-(2-(2-(Benzyloxy)-4,6-dihydroxybenzoyl)isoindolin-4-yl)-4-(dimethylamino)but-2-enamide;
N-(2-(2-(Benzyloxy)-4,6-dihydroxy-benzoyl)isoindolin-4-yl)-2-fluoroacrylamide;
N-(2-(2-(Benzyloxy)-4,6-dihydroxy-3-methylbenzoyl)isoindolin-4-yl)acrylamide;
N-(2-(2-(Benzyloxy)-4,6-dihydroxy-3-methylbenzoyl)isoindolin-4-yl)-2-fluoroacrylamide;
(E)-N-(2-(2-(Benzyloxy)-4,6-dihydroxy-3-methylbenzoyl)isoindolin-4-yl)-4-(dimethylamino)but-2-enamide;
N-(2-(2-(cyclohexylmethoxy)-4, 6-dihydroxy-3-methylbenzoyl) isoindolin-4-yl) acrylamide;
(E)-N-(2-(2-(cyclohexylmethoxy)-4,6-dihydroxy-3-methylbenzoyl)isoindolin-4-yl)-4-(dimethylamino)but-2-enamide;
(E)-N-(2-(2-(Cyclohexylmethoxy)-4,6-dihydroxy-3-methylbenzoyl)isoindolin-4-yl)-4-(pyrrolidin-1-yl)but-2-enamide;
(E)-N-(2-(2-(cyclohexylmethoxy)-4,6-dihydroxy-3-methylbenzoyl)isoindolin-4-yl)-4-morpholinobut-2-enamide hydrochloride;
(E)-N-(2-(2-(Cyclohexylmethoxy)-4,6-dihydroxy-3-methylbenzoyl)isoindolin-4-yl)-4-(4-methylpiperazin-1-yl)but-2-enamide;
N-(2-(2-(Cyclohexylmethoxy)-4,6-dihydroxy-3-methylbenzoyl)isoindolin-4-yl)but-2-ynamide;
(E)-N-(2-(2-(Cyclohexylmethoxy)-4,6-dihydroxy-3-methylbenzoyl)isoindolin-4-yl)but-2-enamide;
(E)-N-[2-[2-(Cyclopentylmethoxy)-4,6-dihydroxy-3-methyl-benzoyl]isoindolin-4-yl]-4-(dimethylamino)but-2-enamide;
(E)-N-[2-(4,6-Dihydroxy-2-methoxy-3-methyl-benzoyl)isoindolin-4-yl]-4-(dimethylamino)but-2-enamide;
N-[2-[2-(Cyclohexylmethoxy)-4,6-dihydroxy-3-methyl-benzoyl]isoindolin-5-yl]prop-2-enamide;
N-[2-[2-(Cyclohexylmethoxy)-4,6-dihydroxy-3-methyl-benzoyl]-3,4-dihydro-1H-isoquinolin-8-yl]prop-2-enamide;
N-[2-[2-(Cyclohexylmethoxy)-4,6-dihydroxy-3-methyl-benzoyl]-3,4-dihydro-1H-isoquinolin-7-yl]prop-2-enamide;
(E)-N-[2-[2-(Cyclohexylmethoxy)-4,6-dihydroxy-3-methyl-benzoyl]isoindolin-5-yl]-4-(dimethylamino)but-2-enamide;
(E)-N-[2-[2-(Cyclohexylmethoxy)-4,6-dihydroxy-3-methyl-benzoyl]-3,4-dihydro-1H-isoquinolin-8-yl]-4-(dimethylamino)but-2-enamide;
(E)-N-[2-[2-(Cyclohexylmethoxy)-4,6-dihydroxy-3-methyl-benzoyl]-3,4-dihydro-1H-isoquinolin-7-yl]-4-(dimethylamino)but-2-enamide;
(E)-4-(Dimethylamino)-N-[2-(2-ethoxy-4,6-dihydroxy-3-methyl-benzoyl)isoindolin-4-yl]but-2-enamide;
(E)-N-[2-[4,6-Dihydroxy-3-methyl-2-(2,2,2-trifluoroethoxy)benzoyl]isoindolin-4-yl]-4-(dimethylamino)but-2-enamide;
(E)-N-[2-[2-(cyclopropylmethoxy)-4,6-dihydroxy-3-methyl-benzoyl]isoindolin-4-yl]-4-(dimethylamino)but-2-enamide;
(E)-N-[2-(4,6-Dihydroxy-2-methoxy-3-methyl-benzoyl)isoindolin-4-yl]-4-(dimethylamino)-N-methyl-but-2-enamide;
(E)-N-[2-(4,6-Dihydroxy-2-methoxy-3-methyl-benzoyl)isoindolin-5-yl]-4-(dimethylamino)but-2-enamide;
(E)-N-[2-(4,6-dihydroxy-2-methoxy-3-methyl-benzoyl)isoindolin-5-yl]-4-(dimethylamino)-N-methyl-but-2-enamide;
(E)-N-[2-(4,6-dihydroxy-2-methoxy-3-methyl-benzoyl)-3,4-dihydro-1H-isoquinolin-7-yl]-4-(dimethylamino)but-2-enamide;
(E)-N-[2-(4,6-dihydroxy-2-methoxy-3-methyl-benzoyl)-3,4-dihydro-1H-isoquinolin-7-yl]-4-(dimethylamino)-N-methyl-but-2-enamide;
(E)-N-[2-[2-(Cyclohexylmethoxy)-4,6-dihydroxy-3-methyl-benzoyl]isoindolin-4-yl]-4-(dimethylamino)-N-methyl-but-2-enamide;
(E)-N-[2-[2-(Cyclopropylmethoxy)-4,6-dihydroxy-3-methyl-benzoyl]isoindolin-4-yl]-4-(dimethylamino)-N-methyl-but-2-enamide;
N-[2-(4,6-dihydroxy-2-methoxy-3-methyl-benzoyl)isoindolin-4-yl]-N-methyl-prop-2-enamide;
(E)-4-(Dimethylamino)-N-(2-(2-ethoxy-4,6-dihydroxy-3-methylbenzoyl)isoindolin-4-yl)-N-methylbut-2-enamide;
(E)-4-[2-(4,6-dihydroxy-2-methoxy-3-methyl-benzoyl)isoindolin-5-yl]oxy-N,N-dimethyl-but-2-enamide;
(E)-4-((2-(4,6-Dihydroxy-2-methoxy-3-methylbenzoyl)isoindolin-4-yl)oxy)-N, N-dimethylbut-2-enamide;
N-(2-(4,6-Dihydroxy-2-methoxy-3-methylbenzoyl) isoindolin-4-yl)-2-((dimethyl amino)methyl) acrylamide;
N-(2-(4,6-Dihydroxy-2-methoxy-3-methylbenzoyl)isoindolin-4-yl)-2-(morpholinomethyl)acrylamide.

Particular compounds of the present invention include any of the compounds exemplified in the present application, or a pharmaceutically acceptable salt or solvate thereof, and, in particular, any of the following:

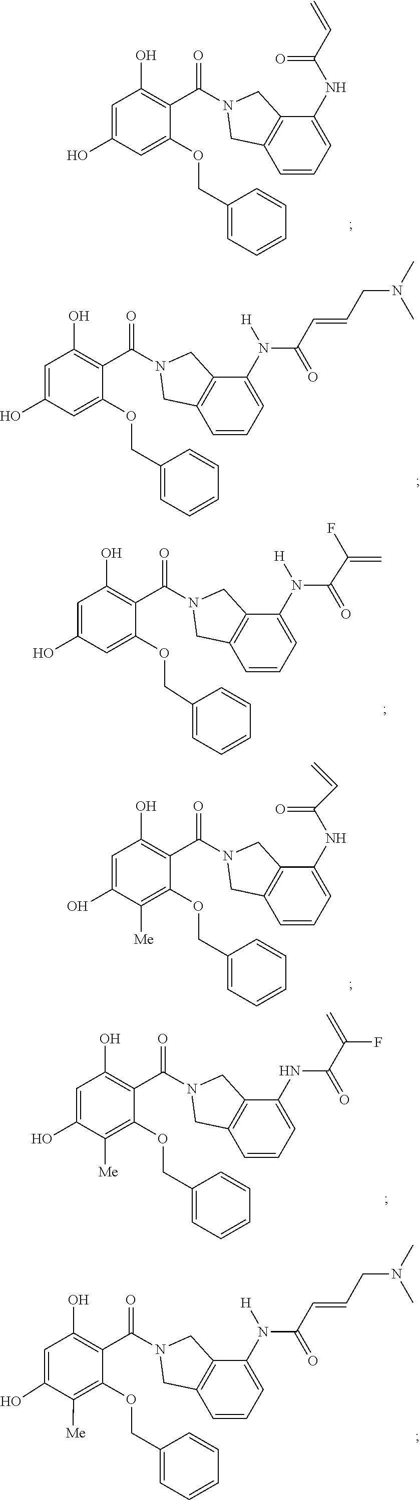

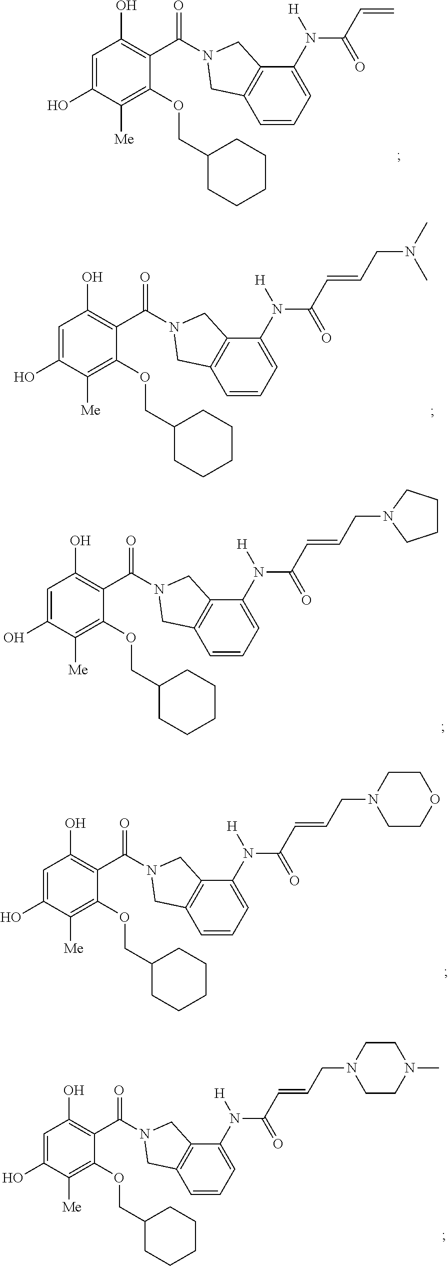 [SMILES: C=CC(=O)Nc1cccc2c1CN(C(=O)c1c(O)cc(O)c(C)c1OCC1CCCCC1)C2 ; Cc1c(O)cc(O)c(C(=O)N2Cc3cccc(NC(=O)/C=C/CN(C)C)c3C2)c1OCC1CCCCC1 ; Cc1c(O)cc(O)c(C(=O)N2Cc3cccc(NC(=O)/C=C/CN4CCCC4)c3C2)c1OCC1CCCCC1 ; Cc1c(O)cc(O)c(C(=O)N2Cc3cccc(NC(=O)/C=C/CN4CCOCC4)c3C2)c1OCC1CCCCC1 ; Cc1c(O)cc(O)c(C(=O)N2Cc3cccc(NC(=O)/C=C/CN4CCN(C)CC4)c3C2)c1OCC1CCCCC1]

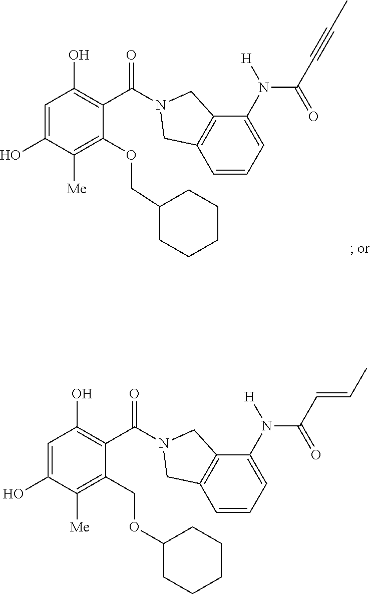 [SMILES: CC#CC(=O)Nc1cccc2c1CN(C(=O)c1c(O)cc(O)c(C)c1OCC1CCCCC1)C2 ; C/C=C/C(=O)Nc1cccc2c1CN(C(=O)c1c(O)cc(O)c(C)c1COC1CCCCC1)C2]

The various functional groups and substituents making up the compounds of the Formula (I) or Formula (II), or sub-formulae (I-I) to (I-XVIII) or II-I to II-VI, are typically chosen such that the molecular weight of the compound of the formula (I) or formula (II) does not exceed 700. More usually, the molecular weight of the compound will be less than 650. More preferably, the molecular weight is less than 600.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z- isomers).

It is to be understood that the present invention encompasses all optical, diastereoisomers and geometric isomers and mixtures thereof that possess activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 160 and 180; and the like.

It is also to be understood that certain compounds of the Formula (I) or Formula (II), or sub-formulae (I-I) to (I-XVIII) or II-I to II-VI, may exist in solvated as well as unsolvated forms such as, for example, hydrated forms. It is to be understood that the invention encompasses all such solvated forms that possess activity.

It is also to be understood that certain compounds of the Formula (I) or Formula (II), or sub-formulae (I-I) to (I-XVIII) or II-I to II-VI, may exhibit polymorphism, and that the invention encompasses all such forms that possess activity.

Compounds of the Formula (I) or Formula (II), or sub-formulae (I-I) to (I-XVIII) or II-I to II-VI, may exist in a number of different tautomeric forms and references to compounds of the Formula (I) or Formula (II), or sub-formulae (I-I) to (I-XVIII) or II-I to II-VI, include all such forms. For the avoidance of doubt, where a compound can exist in one of several tautomeric forms, and only one is specifically described or shown, all others are nevertheless embraced by Formula (I) or Formula (II), or sub-formulae (I-I) to (I-XVIII) or II-I to II-VI. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

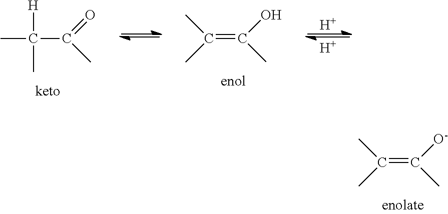

Compounds of the Formula (I) or Formula (II), or sub-formulae (I-I) to (I-XVIII) or II-I to II-VI, containing an amine function may also form N-oxides. A reference herein to a compound of the Formula (I) or Formula (II), or sub-formulae (I-I) to (I-XVIII) or II-I to II-VI, that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid), see for example Advanced Organic Chemistry, by Jerry March, 4th Edition, Wiley Interscience. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I) or Formula (II), or sub-formulae (I-I) to (I-XVIII) or II-I to II-VI, may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I) or Formula (II), or sub-formulae (I-I) to (I-XVIII) or II-I to II-VI, and in-vivo cleavable amide derivatives that may be formed at a carboxy group or an amino group in a compound of the Formula (I) or Formula (II), or sub-formulae (I-I) to (I-XVIII) or II-I to II-VI.

Accordingly, the present invention includes those compounds of the Formula (I) or Formula (II), or sub-formulae (I-I) to (I-XVIII) or II-I to II-VI, as defined hereinbefore, when made available by organic synthesis and when made available within the human or animal body by way of cleavage of a pro-drug thereof. Accordingly, the present invention includes those compounds of the Formula (I) or Formula (II), or sub-formulae (I-I) to (I-XVIII) or II-I to II-VI, that are produced by organic synthetic means and also such compounds that are produced in the human or animal body by way of metabolism of a precursor compound, that is a compound of the Formula (I) or Formula (II), or sub-formulae (I-I) to (I-XVIII) or II-I to II-VI, may be a synthetically-produced compound or a metabolically-produced compound.

A suitable pharmaceutically acceptable pro-drug of a compound of the Formula (I) or Formula (II), or sub-formulae (I-I) to (I-XVIII) or II-I to II-VI, is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I) or Formula (II), or sub-formulae (I-I) to (I-XVIII) or II-I to II-VI, that possesses a carboxy group is, for example, an in vivo cleavable ester thereof. An in vivo cleavable ester of a compound of the Formula I or Formula (II), or sub-formulae (I-I) to (I-XVIII) or II-I to II-VI, containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I) or Formula (II), or sub-formulae (I-I) to (I-XVIII) or II-I to II-VI, that possesses a hydroxy group is, for example, an in vivo cleavable ester or ether thereof. An in vivo cleavable ester or ether of a compound of the Formula (I) or Formula (II), or sub-formulae (I-I) to (I-XVIII) or II-I to II-VI, containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I) or Formula (II), or sub-formulae (I-I) to (I-XVIII) or II-I to II-VI, that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I) or Formula (II), or sub-formulae (I-I) to (I-XVIII) or II-I to II-VI, that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I) or Formula (II), or sub-formulae (I-I) to (I-XVIII) or II-I to II-VI, may be exerted in part by one or more metabolites that are formed within the human or animal body after administration of a compound of the Formula (I) or Formula (II), or sub-formulae (I-I) to (I-XVIII) or II-I to II-VI. As stated hereinbefore, the in vivo effects of a compound of the Formula (I) or Formula (II), or sub-formulae (I-I) to (I-XVIII) or II-I to II-VI, may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae (I-I) to (I-XVIII), will vary depending on the nature of R², R⁴, R⁶, R¹¹, R^{11A}, Y₁, Y₂, A₂, A₃ and A₄ and any substituent groups or subgroups associated therewith. Suitable processes for their preparation are described further in the accompanying Examples.

Once a compound of Formula (I), or sub-formulae (I-I) to (I-XVIII), has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups R², R⁴, R⁶, R¹¹, R^{11A}, Y₁, Y₂, A₂, A₃ and A₄ may be further reacted to change the nature of the group and provide an alternative compound of Formula (I).

The resultant compounds of Formula (I), or sub-formulae (I-I) to (I-XVIII), can be isolated and purified using techniques well known in the art.

The compounds of Formula (I) may be synthesised by the synthetic routes shown in the Examples section below.

### Biological Activity

The biological assays described in the Examples section herein may be used to measure the pharmacological effects of the compounds of the present invention.

Although the pharmacological properties of the compounds of Formula (I) or Formula (II) vary with structural change, as expected, the compounds of the invention were found to be active in a PMS2 in vitro assay as described in the Examples section.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general, lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of PMS2 activity.

The compounds of Formula (I) or Formula (II), or a pharmaceutically acceptable salt thereof, therefore, have potential therapeutic uses in a variety of disease states in which the inhibition of PMS2 activity is beneficial.

The present invention therefore provides a method of treating a disease or disorder in which the inhibition PMS2 activity is beneficial in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of inhibiting PMS2 activity, in vitro or in vivo, said method comprising contacting a cell with an effective amount of a compound or a pharmaceutically acceptable salt, hydrate or solvate thereof as defined herein, or a pharmaceutical composition as defined herein.

The present invention provides a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use as a medicament.

The present invention provides a compound or a pharmaceutically acceptable salt, hydrate or solvate thereof as defined herein, or a pharmaceutical composition as defined herein, for use in the treatment of a proliferative disorder.

The present invention provides a compound, or a pharmaceutically acceptable salt, hydrate or solvate thereof, or a pharmaceutical composition as defined herein for use in the treatment of cancer. In a particular embodiment, the cancer is human cancer. In a particular embodiment, the cancer is human cancer, in particular oestrogen positive cancers, such as breast cancer, or androgen receptor positive cancers, such as prostate cancer.

The present invention provides a compound, or a pharmaceutically acceptable salt, hydrate or solvate thereof, as defined herein, or a pharmaceutical composition as defined herein for use in the inhibition of PMS2 activity.

The present invention provides a compound, or a pharmaceutically acceptable salt, hydrate or solvate thereof, as defined herein, or a pharmaceutical composition as defined herein for use in the treatment of a disease or disorder in which the inhibition of PMS2 activity is beneficial.

The present invention provides a use of a compound, or a pharmaceutically acceptable salt, hydrate or solvate thereof, as defined herein, or a pharmaceutical composition as defined herein in the manufacture of a medicament for the treatment of a proliferative disorder.

The present invention provides a use of a compound, or a pharmaceutically acceptable salt, hydrate or solvate thereof, as defined herein, or a pharmaceutical composition as defined herein in the manufacture of a medicament for the treatment of cancer.

The present invention provides a use of a compound, or a pharmaceutically acceptable salt, hydrate or solvate thereof, as defined herein, or a pharmaceutical composition as defined herein in the manufacture of a medicament for the inhibition of PMS2 activity.

The present invention provides a use of a compound, or a pharmaceutically acceptable salt, hydrate or solvate thereof, as defined herein, or a pharmaceutical composition as defined herein in the manufacture of a medicament for the treatment of a disease or disorder in which the inhibition of PMS2 activity is beneficial.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In particular, the compounds of the present invention will be useful for the treatment of any cancer in which mis-match repair inhibition is beneficial. Any suitable cancer may be targeted (e.g., adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

The compounds of the present invention may also be used to treat triplet repeat disorders.

Thus, a further aspect of the present invention provides a method of treating a triplet repeat disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in the treatment of a triplet repeat disorder. In a particular embodiment, the triplet repeat disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided the use of a compound, or a pharmaceutically acceptable salt, hydrate or solvate thereof, as defined herein, or a pharmaceutical composition as defined herein in the manufacture of a medicament for the treatment of a triplet repeat disorder. In a particular embodiment, the triplet repeat disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery or radiotherapy or chemotherapy or a targeted agent. Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecins including irinotecan);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034);
(vi) vascular damaging agents such as, but not limited to, Combretastatin A₄ and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, ENPP1 inhibitors, CD38 inhibitors, TBK1 inhibitors, A2a receptor antagonists, PI3 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

### Combination Therapy with Immune Modulating Treatments

### Immune Checkpoint Inhibitors

Immune checkpoint proteins present on immune cells and/or cancer cells [e.g. CTLA4 (also known as cytotoxic T-lymphocyte-associated protein 4 and CD152), LAG3 (also known as lymphocyte-activation gene 3 and CD223), PD1 (also known as programmed cell death protein 1 and CD279), PD-L1 (also known as programmed death-ligand 1 and CD274), TIM-3 (also known as T-cell immunoglobulin mucin-3) and TIGIT (also known as T-cell Immunoreceptor with Ig and ITIM domains) are molecular targets that have been found to play an important role in regulating anti-tumour immune responses. Inhibitors of these immune checkpoint proteins (e.g. CTLA4, LAG3, PD1, PD-L1, TIM-3 and/or TIGIT inhibitors) promote an anti-tumour immune response that can be utilised to effectively treat certain forms of cancer.

### Immune Stimulators

Monoclonal antibodies, bispecific antibodies, recombinant ligands and small molecule therapeutics that bind to stimulatory receptors on immune cells can facilitate an effective anti-tumour response. Such receptors may be involved in cell-to-cell contact for example contact between tumour cell and immune cell or between two types of immune cells, other receptors may bind to soluble factors that stimulate an immune response. In one such embodiment antibodies, bispecifics, recombinant proteins or small molecule therapeutics can activate stimulatory receptors, including, but not limited to, 4-1BB, OX40, cGAS-STING, CD27, CD40, and DR3 that enhance anti-tumour immunity.

Modulators of antigen processing may facilitate the presentation of neoantigenic peptides on the cell surface to enhance an effective anti-tumour response. In one such embodiment inhibitors of the endoplasmic reticulum aminopeptidases ERAP1 and ERAP2 may stimulate anti-tumour immunity.

In one aspect, the present invention relates to a combination comprising a compound as defined herein, or a pharmaceutically acceptable salt thereof, and an immune checkpoint inhibitor or immune stimulator as defined herein, or a pharmaceutically acceptable salt thereof, for use in the treatment of a proliferative disorder.

In another aspect, the present invention relates to a use of a combination comprising a compound as defined herein, or a pharmaceutically acceptable salt thereof, and an immune checkpoint inhibitor or immune stimulator as defined herein, or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and an immune checkpoint inhibitor or immune stimulator as defined herein, or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administration with an immune checkpoint inhibitor, or immune stimulator, or a pharmaceutically acceptable salt thereof.

In another aspect, the present invention relates to an immune checkpoint inhibitor or immune stimulator, or a pharmaceutically acceptable salt thereof, for use in the treatment of a proliferative disorder, wherein the immune checkpoint inhibitor is for simultaneous, separate or sequential administration with a compound as defined herein, or a pharmaceutically acceptable salt thereof, as defined herein.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administration with an immune checkpoint inhibitor or immune stimulator, or a pharmaceutically acceptable salt thereof.

In another aspect, the present invention relates to a use of an immune checkpoint inhibitor or immune stimulator, or a pharmaceutically acceptable salt thereof, in the manufacture of a medicament for treating a proliferative disorder, wherein the medicament is for simultaneous, separate or sequential administration with a compound as defined herein, or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and an immune checkpoint inhibitor or immune stimulator as defined herein, or a pharmaceutically acceptable salt thereof, either sequentially, separately or simultaneously.

Any immune checkpoint inhibitor or immune stimulator may be used in the combination therapy defined herein.

In one embodiment, the immune stimulator is selected from a 4-1 BB stimulator, a OX40 stimulator, a CD27 stimulator, a CD40 stimulator, and a DR3 stimulator. In another embodiment the immune checkpoint inhibitor is selected from a PD1-inhibitor, a PD-L1 inhibitor, a LAG3 inhibitor, CTLA-4 inhibitor, a TIM-3 inhibitor and/or a TIGIT inhibitor. In a particular embodiment, the immune checkpoint inhibitor is a PD1 or PD-L1 inhibitor.

PD-1 is a cell surface receptor protein present on immune cells such as T cells. PD-1 plays an important role in down-regulating the immune system and promoting self-tolerance by suppressing T cell activation. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD1 binds two ligands, PD-L1 and PD-L2. PD-L1 is of particular interest as it is highly expressed in several cancers and hence the role of PD1 in cancer immune evasion is well established. Monoclonal antibodies targeting PD-1 that boost the immune system are approved or are being developed for the treatment of cancer. Many tumour cells express PD-L1, an immunosuppressive PD-1 ligand; inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda) and nivolumab (Opdivo). These drugs have been shown to be effective in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (lmfinzi). These drugs have also been shown to be helpful in treating different types of cancer, including bladder cancer, non-small cell lung cancer, and Merkel cell skin cancer (Merkel cell carcinoma). They are also being studied for use against other types of cancer.

Examples of LAG3 inhibitors include BMS-986016/Relatlimab, TSR-033, REGN3767, MGDO13 (bispecific DART binding PD-1 and LAG-3), GSK2831781 and LAG525.

Examples of CTLA-4 inhibitors include MDX-010/ipilimumab, AGEN1884, and CP-675,206/Tremelimumab.

Examples of TIM-3 inhibitors include MBG453 (Novartis), TSR-022 (Tesaro), and LY3321367 (Lilly).

Examples of TIGIT inhibitors include Tiragolumab (MTIG7192A; RG6058; Genentech/Roche), AB154 (Arcus Bioscience), MK-7684 (Merck), BMS-986207 (Bristol-Myers Squibb), ASP8374 (Astellas Pharma; Potenza Therapeutics).

In one embodiment, the immune checkpoint inhibitor is selected from BMS-986016/Relatlimab, TSR-033, REGN3767, MGD013 (bispecific DART binding PD-1 and LAG-3), GSK2831781, LAG525, MDX-010/ipilimumab, AGEN1884, and CP-675,206/Tremelimumab, pembrolizumab, nivolumab, atezolizumab, avelumab, durvalumab, MBG453, TSR-022, LY3321367, Tiragolumab (MTIG7192A; RG6058), AB154, MK-7684, BMS-986207, and/or ASP8374 or a pharmaceutically acceptable salt or solvate thereof.

### Combination Therapy with DNA Damage Response Modulators

The compounds of the present invention are particularly suited to use in combination with agents that act as DNA damage response modulators, e.g. PARP inhibitors, ATM inhibitors and ATR inhibitors.

In one aspect, the present invention relates to a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, for use in the treatment of a proliferative disorder.

In another aspect, the present invention relates to a use of a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administration with a DNA damage response modulator (e.g. a PARP inhibitor, an ATM inhibitor and/or an ATR inhibitor), or a pharmaceutically acceptable salt thereof.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administration with a DNA damage response modulator (e.g., a PARP inhibitor, an ATM inhibitor and/or an ATR inhibitor), or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and a DNA damage response modulator (e.g., a PARP inhibitor, an ATM inhibitor and/or an ATR inhibitor), or a pharmaceutically acceptable salt thereof, either sequentially, separately or simultaneously.

Any DNA damage response modulator (e.g., a PARP inhibitor, an ATM inhibitor and/or an ATR inhibitor) may be used in the combination therapy defined herein.

### EXAMPLES

While specific embodiments of the invention have been described herein for the purpose of reference and illustration, various modifications will be apparent to a person skilled in the art without departing from the scope of the invention as defined by the appended claims.

### Abbreviations

Boc for tert-butyloxycarbonyl
DAST for diethylaminosulfur trifluoride
DBU for 1,8-diazabicyclo(5.4.0)undec-7-ene
DCC for dicyclohexylcarbodiimide
DCE for 1,1-dichloroethane
DCM for dichloromethane
DEA for diethanolamine
DEAD for diethyl azodicarboxylate
DIAD for diisopropyl azodicarboxylate
DIBAL for Diisobutylaluminium hydride
DIPEA for N,N-diisopropylethylamine, Hünig's base
DMA for N,N-dimethylacetamide
DMAP for 4-(dimethylamino) pyridine
DMF for N,N-dimethylformamide
DMSO for dimethylsulfoxide.
EDC for 1-Ethyl-3-(3-dimethylaminopropyl)carbodiimide
EtOAc for ethyl acetate
h for hours
HATU for N-[(dimethylamino)-1H-1,2,3-triazolo-[4,5-b]pyridin-1-ylmethylene]-N-methylmethanaminium hexafluorophosphate N-oxide
HBTU for (1H-benzotriazol-1-yloxy)(dimethylamino)-N,N-dimethylmethaniminium hexafluorophosphate
HOBT for N-hydroxybenzotriazole
HPLC for High Pressure Liquid Chromatography.
LAH for lithium aluminium hydride
IPA for isopropyl alcohol
LCMS for Liquid Chromatography-Mass Spectrometry
LDA for Lithium diisopropylamide
LiHMDS for Lithium bis(trimethylsilyl)amide
mCPBA for meta-chloroperoxybenzoic acid
MI for Molecular Ion
Min for minutes
MgSO₄ anhydrous magnesium sulfate
MW for microwave
NBS for N-bromosuccinamide
NCS for N-chlorosuccinamide
NFOBS for N-fluoro-o-benzenedisulfonimide
NFSI for N-fluorobenzenesulfonimide
NHS for N-hydroxysuccinimide
NIS for N-iodosuccinamide
NMM for N-methylmorpholine
NMP for 1-methyl-2-pyrrolidinone
NMR for Nuclear Magnetic Resonance.
PdCl₂(PPh₃)₂ for Bis(triphenylphosphine)palladium chloride
Pd(dppf)₂Cl₂ for [1,1′-bis(diphenylphosphino)ferrocene]dichloropalladium(II)
Pd(dppf)₂Cl₂.DCM for [1,1′-bis(diphenylphosphino)ferrocene]dichloropalladium(II) complex with DCM
(Pd(dba)₂) for bis(dibenzylideneacetone)palladium
Rbf for round bottomed flask
RT for Retention Time.
SCX-2 for a silica-based sorbent with a chemically bonded propylsulfonic acid functional group
SFC for supercritical fluid chromatography
TBAF for tetra-n-butylammonium fluoride
TBDMS for tert-butyldimethylsilyl
TFAA for trifluoroacetic anhydride
TFA for trifluoroacetic acid
THF for tetrahydrofuran
T3P for Propylphosphonic anhydride
Ts for p-toluenesulfonyl
XPhos-Pd-G1 for 2-Dicyclohexylphosphino-2′,4′,6′-triisopropyl-1,1′-biphenyl)[2-(2-aminoethyl)phenyl)]palladium(II) chloride
XPhos-Pd-G2 for Chloro(2-dicyclohexylphosphino-2′,4′,6′-triisopropyl-1,1′-biphenyl)[2-(2′-amino-1,1′-biphenyl)]palladium(II)

### Analytical Methods

Commercially available starting materials, reagents and dry solvents were used as supplied. Flash column chromatography or glass column chromatography was performed using Merck silica gel 230-400 mesh size. Flash chromatography was also performed on combi-flash RF Teledyne Isco machine. Preparative TLC was performed on Merck plates.

### Liquid Chromatography-Mass Spectrometry Methods

### Method-A

Waters Acquity UPLC with binary solvent manager, PDA detector and Acquity QDA performance mass detector, column: X-Bridge BEH C18, 50×2.1 mm, 2.5 micron, column temperature: 35° C., auto sampler temperature: 5° C., mobile phase A: 0.1% (v/v) formic acid in water (pH=2.70), Mobile Phase B: 0.1% formic acid (v/v) in water: acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method-D

Waters Acquity UPLC with quaternary solvent manager, with PDA detector and SQ detector, column: X-Bridge BEH C18, 50*2.1 mm, 2.5 micron, column temperature: 35° C., auto sampler temperature: 5° C., mobile phase A: 0.1% (v/v) Formic acid in water (pH=2.70), mobile phase B: 0.1% (v/v) formic acid in water: acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ESI capillary probe, ionization mode cycled through positive and negative modes with cone voltage 10 V and 30V and 0.8 kV capillary voltage, temperature of source and probe were 120° C. and 400° C. respectively.

### Method-E

Waters Acquity UPLC with binary solvent manager, PDA detector and Acquity QDA performance mass detector, column: Welch Xtimate C18, 50*2.1 mm, 1.8 micron, column temperature: 35° C., auto sampler temperature: 5° C., mobile phase A: 0.1% (v/v) formic acid in water (pH=2.70), Mobile Phase B: 0.1% formic acid (v/v) in water: acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method-H

Waters 996 Photodiode Array Detector equipped with Waters Micromass ZQ detector, column: XTIMATE C18 5 μm 4.6*150 mm, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: −1.0 mL/min, Run Time: −17 min, UV Detection Method: −PDA. Mass parameter: Probe: ESI, Mode of Ionisation: Positive and Negative, Cone voltage: −30 and 10 V, capillary voltage: −3.0 KV, Extractor Voltage: −2 V, Rf Lens: −0.1 V, Temperature of source: −120° C., Temperature of Probe: −400° C., Cone Gas Flow: −100 L/Hr, Desolvation Gas flow: −800 L/Hr.

### Preparative HPLC Methods

### Method A

Shimadzu LC20AP purification system with UV detector and YMC-Actus Triart prep 250×20 mm, 5 μm column. Compounds were eluted with, Mobile Phase A: 0.1% Formic Acid in Milli Q water, Mobile Phase B: Acetonitrile; gradient T=0 min (63% A, 37% B); gradient to T=20 min (45% A, 55% B); T=20.01 min (2% A, 98% B); gradient to T=24 min (2% A, 98% B); T=24.01 min (63% A, 37% B) to T=28 min (63% A, 37% B);); Flow rate=16 ml/min; analysis time 28 min.

### Method B

Buchi Prep Chrom C700 with UV Detector system using a YMC120 gm C18, 50 μm column. Compounds were eluted with: Mobile Phase A: 0.1% Formic Acid in Milli Q water, Mobile Phase B: Acetonitrile; gradient T=0 min (100% A, 0% B); gradient to T=30 min (50% A, 50% B); T=30.01 min (2% A, 98% B); gradient to T=35 min (2% A, 98% B); T=35.01 min (100% A, 0% B) to T=40 min (100% A, 0% B); Flow rate=80 ml/min. analysis time 40 min.

### Method C

Waters 2545 System with UV detector using X-Bridge Prep, C18, OBD (250×19) mm, 5 μm column. Compounds eluted with: Mobile Phase A: 5 mM ammonium bicarbonate in 0.05% ammonia/water., Mobile Phase B: Acetonitrile; gradient of T=0 min (58% A, 42% B); gradient to T=22 min (2% A, 98% B); T=22.01 min (2% A, 98% B); T=25 min (58% A, 42% B). Flow rate=16 ml/min.

### Method D

Waters 2545 System with UV detector using X-Bridge Prep, C18, OBD (250×19) mm, 5 μm column. Compounds eluted with: Mobile Phase A: 0.05% ammonia in water, Mobile Phase B: Acetonitrile; gradient T=0 min (85% A, 15% B); gradient to T=17 min (68% A, 32% B); T=17.01 min (2% A, 98% B); T=19 min (85% A, 15% B). Flow rate=21 ml/min.

### Method E

Waters 2545 System with UV detector using YMC-ODS AQ C18 (250×20) mm, 5 μm column. Compounds eluted with: Mobile Phase A: 0.1% formic acid in water, Mobile Phase B: 20% A in Acetonitrile; gradient T=0 min (75% A, 25% B); gradient to T=19 min (60% A, 40% B); T=19.01 min (2% A, 98% B); T=25 min (75% A, 25% B). Flow rate=17 ml/min.

### Preparation of Compounds

### N-(2-(2-(Benzyloxy)-4,6-dihydroxybenzoyl)isoindolin-4-yl)acrylamide: Example 1

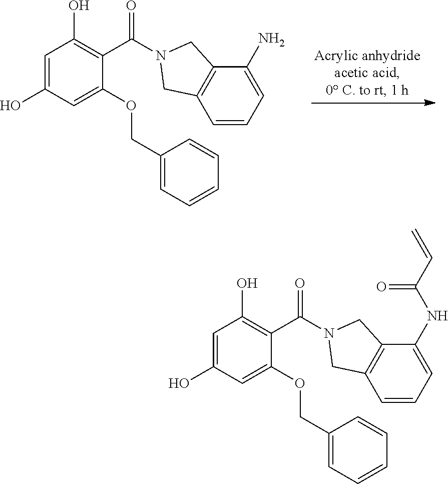 [SMILES: Nc1cccc2c1CN(C(=O)c1c(O)cc(O)cc1OCc1ccccc1)C2 ; C=CC(=O)Nc1cccc2c1CN(C(=O)c1c(O)cc(O)cc1OCc1ccccc1)C2]

A stirred solution of (4-aminoisoindolin-2-yl)(2-(benzyloxy)-4,6-dihydroxyphenyl)methanone (Intermediate 1) (0.2 g, 0.53 mmol, 1 eq.) in AcOH (5 mL) at 0° C. was treated with acrylic anhydride (CAS 2051-76-5, 0.067 g, 0.53 mmol, 1.0 eq.). The reaction mixture was stirred at room temperature for 1 h, poured into ice cold water (5 mL) and neutralized using sat. NaHCO₃ solution and extracted in ethyl acetate (3×50 mL). The combined organic layer was washed with brine solution (200 mL), dried over Na₂SO₄, filtered, and concentrated under vacuum. Crude material was purified by column chromatography (product eluted with 1.7% MeOH in DCM) yielding the title compound as an off white solid (0.085 g, Yield: 37%).

¹H NMR (DMSO-d6, 400 MHz): δ 4.50 (bs, 2H), 4.71-4.75 (m, 2H), 5.03 (s, 2H), 5.70-5.80 (m, 1H), 5.99-6.01 (m, 2H), 6.19-6.31 (m, 1H), 6.42-6.60 (m, 1H), 7.04 (d, J=7.6 Hz, 1H), 7.15-7.31 (m, 6H), 7.62-7.65 (m, 1H), 9.51 (bs, 1H), 9.65 (bs, 1H), 9.78 (bs, 1H). LCMS (Method A) 1.538 min, MS ES+431.17 (M+1).

### (E)-N-(2-(2-(Benzyloxy)-4,6-dihydroxybenzoyl)isoindolin-4-yl)-4-(dimethylamino)but-2-enamide: Example 2

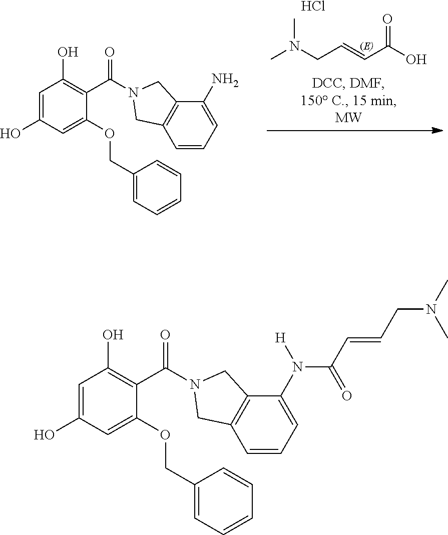

A stirred solution of (4-aminoisoindolin-2-yl)(2-(benzyloxy)-4,6-dihydroxyphenyl)methanone (Intermediate 1) (0.2 g, 0.52 mmol, 0.3 eq.) and (E)-4-(dimethylamino)but-2-enoic acid hydrochloride (CAS 848133-35-7; 0.292 g, 1.75 mmol, 1.0 eq.) in DMF (0.3 mL) in a 10 mL microwave tube at room temperature was treated with DCC (0.364 g, 1.75 mmol, 1 eq.). The reaction mixture was heated to 150° C. for 15 min under microwave conditions. The reaction mixture was poured into ice cold water (70 mL) and washed with ethyl acetate (3×50 mL; product is water soluble). The aqueous layer was concentrated under vacuum to obtain crude material (0.60 g). The crude material was purified by Prep HPLC (Method B) yielding the title compound as an off-white solid (0.097 g, Yield: 26%).

¹H NMR (DMSO-d6, 400 MHz): δ 2.15 (s, 3H), 2.20 (s, 3H), 3.03-3.09 (dd, J=4.8, 20 Hz, 2H), 4.51 (s, 2H), 4.74 (d, J=12.8 Hz, 2H), 5.04 (s, 2H), 6.00-6.01 (m, 2H), 6.29-6.33 (m, 1H), 6.41-6.45 (m, 1H), 6.67-6.78 (m, 1H), 7.02-7.31 (m, 7H), 7.68 (d, J=8 Hz, 1H), 9.53 (s, 1H), 9.60 (s, 1H), 9.68 (s, 1H). LCMS (Method A): 1.221 min, MS: ES+488.1 (M+1).

### N-(2-(2-(Benzyloxy)-4,6-dihydroxy-benzoyl)isoindolin-4-yl)-2-fluoroacrylamide: Example 3

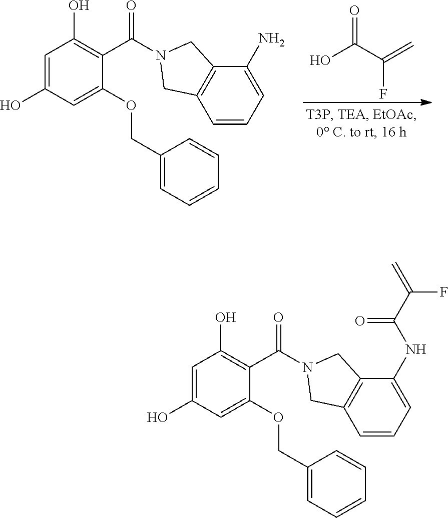

A stirred solution of (4-aminoisoindolin-2-yl)(2-(benzyloxy)-4,6-dihydroxyphenyl)methanone (Intermediate 1) (0.1 g, 0.26 mmol, 1 eq.) in ethyl acetate (2 mL) was cooled to 0° C.; molecular sieves (0.05 g), TEA (0.214 g, 2.14 mmol, 8 eq.), 2-fluoroacrylic acid (CAS: 430-9-90; 047 g, 0.53 mmol, 2 eq.) and T3P (50% in ethyl acetate) (0.25 mL, 0.79 mmol, 3.0 eq.) were added. The resulting reaction mixture was stirred at room temperature for 16 h. The resulting reaction mixture was poured into water (25 mL) and extracted with ethyl acetate (3×30 mL). The combined organic layer was dried over Na₂SO₄, filtered, and concentrated under reduced pressure. Crude material was purified by flash chromatography (product eluted at 5% methanol in DCM) yielding the title compound (0.022 g, Yield: 9.3%).

¹H NMR (DMSO-d6, 400 MHz): δ 4.47-4.52 (m, 2H), 4.67-4.77 (m, 2H), 5.03 (s, 2H), 5.35-5.39 (m, 1H), 5.58-5.79 (m, 1H), 5.98-6.01 (m, 2H), 7.22-7.36 (m, 8H), 9.50-9.59 (m, 2H), 10.15-10.26 (m, 1H). LCMS (Method A): 1.579 min, MS ES+449.20 (M+1).

### N-(2-(2-(Benzyloxy)-4,6-dihydroxy-3-methylbenzoyl)isoindolin-4-yl)acrylamide: Example 4

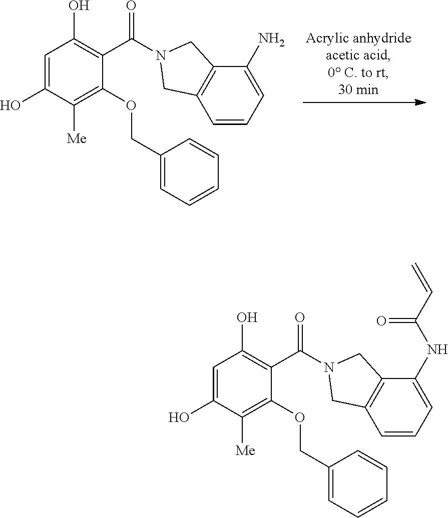 [SMILES: Cc1c(O)cc(O)c(C(=O)N2Cc3cccc(N)c3C2)c1OCc1ccccc1 ; C=CC(=O)Nc1cccc2c1CN(C(=O)c1c(O)cc(O)c(C)c1OCc1ccccc1)C2]

A stirred solution of (4-aminoisoindolin-2-yl)(2-(benzyloxy)-4,6-dihydroxy-3-methylphenyl) methanone (Intermediate 2) (0.2 g, 0.51 mmol, 1 eq.) in AcOH (0.7 mL) at 0° C. was treated with acrylic anhydride (0.071 g, 0.56 mmol, 1.1 eq.). The reaction mixture was stirred at room temperature for 30 min, then neutralized using sat. NaHCO₃ (pH=6-7) and extracted into ethyl acetate (3×50 mL). The combined organic layer was dried over Na₂SO₄, filtered, and concentrated under vacuum. The crude material was purified by column chromatography (product eluted with 2.4% MeOH in DCM) to give the title compound as an off white solid (0.070 g, 0.16 mmol. Yield: 30.7%).

¹H NMR (DMSO-d6, 400 MHz): δ 1.97 (s, 3H), 4.44-4.50 (m, 1H), 4.57-4.61 (m, 1H), 4.72-4.78 (m, 3H), 4.90-4.93 (m, 1H), 5.69-5.82 (m, 1H), 6.18-6.32 (m, 2H), 6.41-6.59 (m, 1H), 7.02-7.18 (m, 1H), 7.23-7.34 (m, 6H), 7.66 (d, J=8.0 Hz, 1H), 9.52 (d, J=6.0 Hz, 1H), 9.56 (s, 1H), 9.78 (s, 1H). LCMS (Method A): 1.756 min, MS: ES+445.2. Analytical HPLC Method A: 6.425 min, 210 nm, 95.1%.

### N-(2-(2-(Benzyloxy)-4,6-dihydroxy-3-methylbenzoyl)isoindolin-4-yl)-2-fluoroacrylamide: Example 5

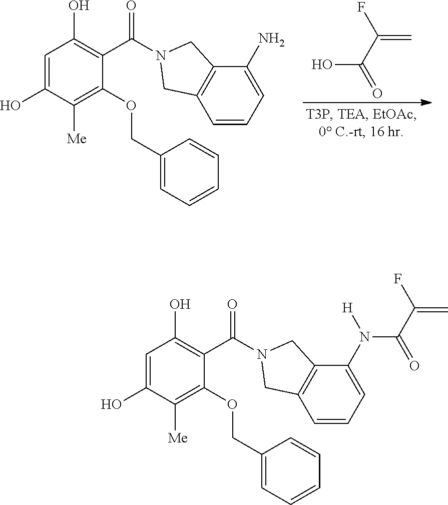

A stirred solution of (4-aminoisoindolin-2-yl)(2-(benzyloxy)-4,6-dihydroxy-3-methylphenyl)methanone (Intermediate 2) (0.080 g, 0. 0.20 mmol, 1.0 eq.) in ethyl acetate (2 mL) was cooled to 0° C. and treated with molecular sieves (0.5 g), TEA (0.166 g, 1.64 mmol, 8 eq.), 2-fluoroacrylic acid (0.036 g, 0.41 mmol, 2 eq.) (CAS: 430-9-9) and T3P (50% in EtOAc) (0.39 g, 0.61 mmol, 3.0 eq.). The resulting reaction mixture was stirred at room temperature for 16 h, then poured into water (25 mL) and extracted with ethyl acetate (3×30 mL). The combined organic layer was dried over Na₂SO₄, filtered, and concentrated under reduced pressure. The obtained crude material was purified by prep. TLC (5% methanol in DCM) yielding the title compound (0.010 g, Yield: 10.6%).

¹H NMR (DMSO-d6, 400 MHz): Compound is a mixture of rotamers: 1.97 (s, 3H), 4.45-4.54 (m, 2H), 4.59-4.79 (m, 3H), 4.90-4.93 (m, 1H), 5.36-5.40 (m, 2H), 5.49-5.70 (m, 1H), 6.31 (2 singlets, 1H), 7.15-7.37 (m, 8H), 9.56-9.59 (m, 2H, D₂O exchangeable), 10.12-10.29 (bs, 1H, D₂O exchangeable). LCMS (Method A): 1.798 min, MS: ES+463.17 (M+1).

### (E)-N-(2-(2-(Benzyloxy)-4,6-dihydroxy-3-methylbenzoyl)isoindolin-4-yl)-4-(dimethylamino)but-2-enamide: Example 6

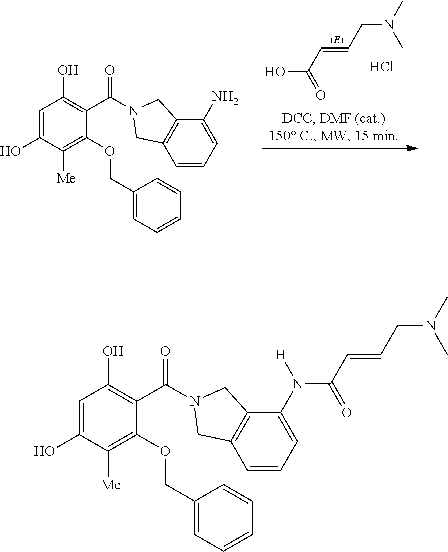

A stirred solution (E)-4-(dimethylamino)but-2-enoic acid hydrochloride (CAS 848133-35-7; 0.141 g, 0.85 mmol, 1 eq.) and 4-(4-aminoisoindoline-2-carbonyl)-5-(benzyloxy)-6-methyl-1,3-phenylene bis(4-methylbenzenesulfonate) (Intermediate 2) (0.1 g, 0.25 mmol, 0.3 eq.) in a microwave vial at room temperature was treated with DCC (0.212 g, 1.03 mmol, 2 eq.) and DMF (0.1 mL). The resulting reaction mixture was heated to 150° C. and stirred for 15 min under microwave irradiation. The reaction mixture was poured into ice cold water (100 mL) and extracted using ethyl acetate (3×50 mL). The desired product was water soluble, and the aqueous layer was concentrated under vacuum. The crude was purified by Prep HPLC (Method A). The pure fractions were lyophilized yielding the title compound as an off white solid (0.030 g, Yield: 16.7%).

¹H NMR (DMSO-d6, 400 MHz, D₂O exchange): δ 1.97 (s, 3H), 2.23 (s, 3H), 2.32 (s, 3H), 3.04-3.05 (m, 1H), 3.11-3.13 (m, 1H), 4.40-4.57 (m, 2H), 4.70-4.80 (m, 3H), 4.86-4.89 (m, 1H), 6.27 (s, 1H), 6.38-6.42 (m, 1H), 6.62-6.80 (m, 1H), 7.01-7.24 (m, 1H), 7.24-7.33 (m, 6H), 7.55 (d, J=20 Hz, 1H); LCMS (Method A): 1.331 min, 95.14%, 254 nm MS: ES+502.4

### N-(2-(2-(Cyclohexylmethoxy)-4, 6-dihydroxy-3-methylbenzoyl) isoindolin-4-yl) acrylamide: Example 7

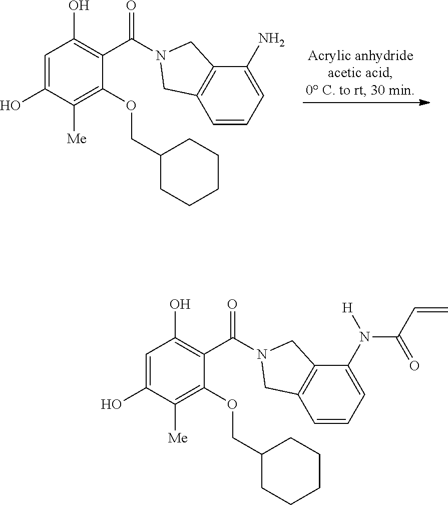 [SMILES: Cc1c(O)cc(O)c(C(=O)N2Cc3cccc(N)c3C2)c1OCC1CCCCC1 ; C=CC(=O)Nc1cccc2c1CN(C(=O)c1c(O)cc(O)c(C)c1OCC1CCCCC1)C2]

A stirred solution of (4-aminoisoindolin-2-yl)(2-(cyclohexylmethoxy)-4,6-dihydroxy-3-methylphenyl)methanone (Intermediate 3) (0.08 g, 0.2 mmol, 1 eq.) in AcOH (0.8 mL) at 0° C. was treated with acrylic anhydride (0.02 mL, 0.16 mmol, 1 eq.). The reaction mixture was stirred at room temperature for 30 min., neutralized with sat. NaHCO₃ solution and extracted in ethyl acetate (3×50 mL). The combined organic layer was dried over Na₂SO₄, filtered, and concentrated under vacuum. The crude material was purified by preparative TLC (5% MeOH in DCM as a solvent system) yielding the title compound as an off white solid (0.038 g, Yield: 33.4%).

¹H NMR (DMSO-d6, 400 MHz): δ 0.95-0.97 (m, 2H), 1.07-1.26 (m, 3H), 1.55-1.68 (m, 6H), 1.95 (s, 3H), 3.53-3.67 (m, 2H), 4.56 (s, 2H), 4.67-4.82 (m, 2H), 5.67-5.77 (m, 1H), 6.18-6.31 (m, 2H), 6.55-6.59 (m, 1H), 7.04, 7.20 (dd, J=7.6 Hz, 1H), 7.23-7.28 (m, 1H), 7.63 (d, J=8 Hz, 1H), 9.06-9.15 (m, br, 2H), 9.45-9.57 (m, br, 1H). LCMS (Method A): 1.957 min, MS: ES+451.4 (M+1). Analytical HPLC Method C [6.751 min, 254 nm, 95.4%.

### (E)-N-(2-(2-(cyclohexylmethoxy)-4,6-dihydroxy-3-methylbenzoyl)isoindolin-4-yl)-4-(dimethylamino)but-2-enamide: Example 8

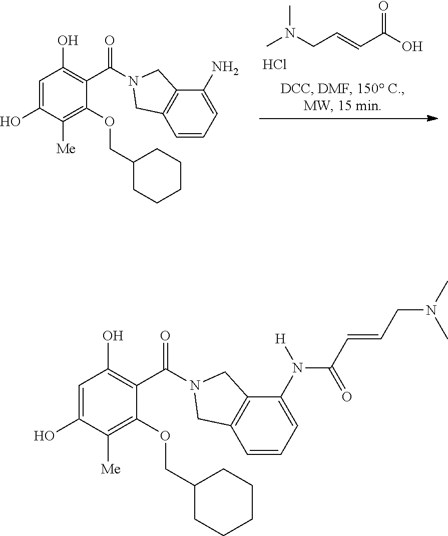

Carried out in two parallel batches at 0.15 g scale: A stirred solution of (E)-4-(dimethylamino) but-2-enoic acid hydrochloride (CAS 848133-35-7; 0.15 g, 0.90 mmol, 1 eq.) and (4-aminoisoindolin-2-yl) (2-(cyclohexylmethoxy)-4, 6-dihydroxy-3-methylphenyl) methanone (Intermediate 3) (0.10 g, 0.27 mmol, 0.3 eq.) in DMF (0.05 mL) in a 10 mL microwave tube at room temperature was treated with DCC (0.37 g, 1.8 mmol, 2 eq.); the reaction mixture was heated to 150° C. and stirred for 15 min under microwave irradiation. The reaction mixture was diluted with ethyl acetate (50 mL) and washed with water (60 mL). The aqueous layer was further washed with ethyl acetate (3×30 mL) and the aqueous layer concentrated under reduced pressure. The crude material was purified by Prep HPLC (Method B) yielding the title compound as an off white solid (0.045 g, Yield: 13.0%).

¹H NMR (DMSO-d6, 400 MHz,): δ 1.04-1.16 (m, 3H), 1.58-1.68 (m, 8H), 1.90-1.92 (s, 3H), 2.15 (s, 3H), 2.20 (s, 3H), 3.01-3.08 (m, 2H), 3.15-3.19 (m, 2H), 3.50-3.51 (m, 1H), 3.65 (t, J=7.2 Hz, 1H), 4.53 (s, 2H), 4.68-4.75 (m, 2H), 6.25 (s, 1H) 6.25-6.47 (m, 1H), 6.66-6.78 (m, 1H), 7.03-7.17 (m, 1H), 7.23-7.30 (m, 1H), 7.71 (d, J=7.6 Hz, 1H), 9.43 (s, 1H), 9.51-9.53 (m, 1H). LCMS (Method A): 1.511 min, MS: ES+508.13 (M+1). Analytical HPLC Method A: 4.933 min 97.5%.

### (E)-N-(2-(2-(Cyclohexylmethoxy)-4,6-dihydroxy-3-methylbenzoyl)isoindolin-4-yl)-4-(pyrrolidin-1-yl)but-2-enamide: Example 9

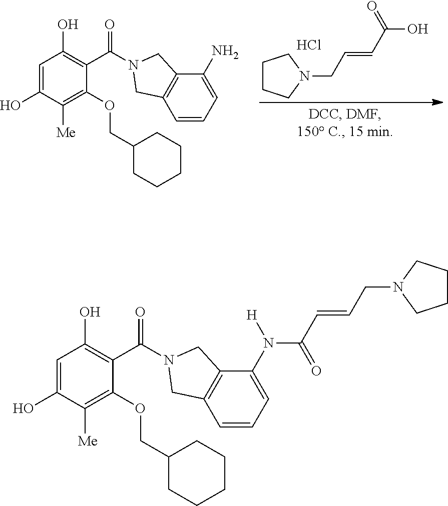 [SMILES: Cc1c(O)cc(O)c(C(=O)N2Cc3cccc(N)c3C2)c1OCC1CCCCC1 ; O=C(O)/C=C/CN1CCCC1 ; Cl ; Cc1c(O)cc(O)c(C(=O)N2Cc3cccc(NC(=O)/C=C/CN4CCCC4)c3C2)c1OCC1CCCCC1]

A stirred solution of (E)-4-(pyrrolidin-1-yl)but-2-enoic acid hydrochloride (CAS 848185-03-05; 0.263 g, 1.26 mmol, 1 eq.), (4-aminoisoindolin-2-yl)(2-(cyclohexylmethoxy)-4,6-dihydroxy-3-methylphenyl)methanone (Intermediate 3) (0.150 g, 0.38 mmol, 0.3 eq.) in DMF (0.26 mL) at room temperature was treated with DCC (0.260 g, 1.26 mmol, 1 eq.). The reaction mixture was heated to 150° C. and stirred for 15 min., cooled, poured into ice cold water (50 mL) and extracted using ethyl acetate (3×50 mL). The desired product was water soluble. The aqueous layer was evaporated to give crude material (0.480 g) which was purified by Prep HPLC (Method C) followed by lyophilization yielding the title compound as an off white solid (0.045 g, Yield: 14.6%).

¹H NMR (DMSO-d6, 400 MHz): δ ppm 0.70-0.99 (m, 2H), 1.05-1.16 (m, 3H), 1.58-1.65 (m, 5H), 1.69-1.73 (m, 5H), 1.92 and 1.93 (2 singlets, 3H), 2.44-2.46 (m, 2H), 3.18-3.26 (m, 2H), 3.50-3.52 (m, 1H), 3.63-3.67 (m, 1H), 4.53 (bs, 2H), 4.72-4.75 (m, 2H), 6.25 (s, 1H), 6.29-6.47 (m, 1H), 6.71-6.83 (m, 1H), 7.03-7.17 (m, 1H), 7.23-7.28 (m, 1H), 7.69 (t, J=7.6 Hz, 1H), 9.45-9.66 (m, br, 2H). LCMS (Method A): 1.536 min, MS: ES+534.2 (M+1). Analytical HPLC (Method B): 5.540 min. 98.9%, 254 nm.

### (E)-N-(2-(2-(cyclohexylmethoxy)-4,6-dihydroxy-3-methylbenzoyl)isoindolin-4-yl)-4-morpholinobut-2-enamide hydrochloride: Example 10

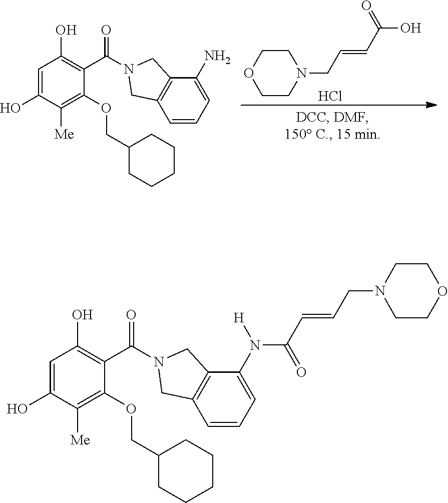 [SMILES: Cc1c(O)cc(O)c(C(=O)N2Cc3cccc(N)c3C2)c1OCC1CCCCC1 ; O=C(O)/C=C/CN1CCOCC1 ; Cl ; Cc1c(O)cc(O)c(C(=O)N2Cc3cccc(NC(=O)/C=C/CN4CCOCC4)c3C2)c1OCC1CCCCC1]

A stirred solution of (E)-4-morpholinobut-2-enoic acid hydrochloride (CAS 1807940-64-2; 0.263 g, 1.26 mmol, 1 eq.), (4-aminoisoindolin-2-yl)(2-(cyclohexylmethoxy)-4,6-dihydroxy-3-methylphenyl)methanone (Intermediate 3) (0.150 g, 0.38 mmol, 0.3 eq.) in DMF (0.26 mL) at room temperature was treated with DCC (0.260 g, 1.26 mmol, 1 eq.). The reaction mixture was heated to 150° C. and stirred for 15 min, cooled, poured into ice cold water (50 mL) and extracted using ethyl acetate (3×50 mL). The combined organic layer was dried over Na₂SO₄ and concentrated under vacuum. The crude was purified by Prep HPLC (Method D) followed by lyophilization yielding the title compound as an off white solid (0.037 g, Yield: 10.2%).

¹H NMR (DMSO-d6, 400 MHz): δ ppm 0.80-0.99 (m, 2H), 1.07-1.16 (m, 3H), 1.58-1.68 (m, 6H), 1.92 and 1.923 (2 singlets, 3H), 2.33-2.36 (m, 2H), 2.47 (m, br, 2H), 3.07-3.15 (m, 2H), 3.50-3.51 (m, 1H), 3.57-3.64 (m, 5H), 4.52-4.56 (m, 2H), 4.68-4.79 (m, 2H), 6.32 (s, 1H), 6.42-6.47 (m, 1H), 6.64-6.79 (m, 1H), 7.03-7.17 (m, 1H), 7.23-7.30 (m, 1H), 7.67 (d, J=8 Hz, 1H), 9.40-9.47 (m, 2H), 9.55-9.68 (m, 1H). LCMS (Method H): 8.646 min, MS: ES+550.7 (M+1). Analytical HPLC (Method B): 5.407 min. 95.04%, 254 nm.

### (E)-N-(2-(2-(Cyclohexylmethoxy)-4,6-dihydroxy-3-methylbenzoyl)isoindolin-4-yl)-4-(4-methylpiperazin-1-yl)but-2-enamide: Example 11

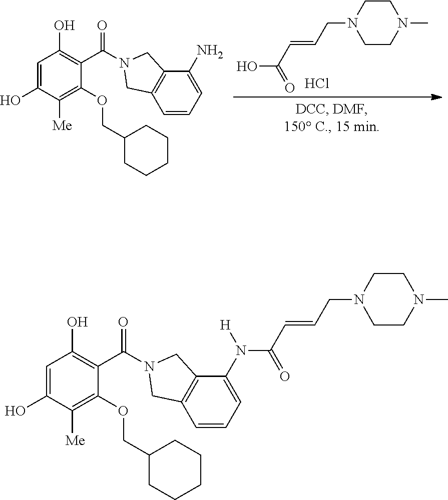 [SMILES: Cc1c(O)cc(O)c(C(=O)N2Cc3cccc(N)c3C2)c1OCC1CCCCC1 ; CN1CCN(C/C=C/C(=O)O)CC1 ; Cl ; Cc1c(O)cc(O)c(C(=O)N2Cc3cccc(NC(=O)/C=C/CN4CCN(C)CC4)c3C2)c1OCC1CCCCC1]

A stirred solution of (E)-4-(4-methylpiperazin-1-yl)but-2-enoic acid (CAS 1472802-56-4: 0.250 g, 1.13 mmol, 1 eq.), (4-aminoisoindolin-2-yl)(2-(cyclohexylmethoxy)-4,6-dihydroxy-3-methylphenyl)methanone (Intermediate 3) (0.130 g, 0.33 mmol, 0.3 eq.) in DMF (0.28 mL) at room temperature was treated with DCC (0.261 g, 1.27 mmol, 1 eq.). The reaction mixture was heated to 150° C. and stirred for 15 min, cooled, poured into ice cold water (50 mL) and extracted using ethyl acetate (3×50 mL). Product was observed in both the organic and aqueous layers. The combined organic and aqueous layers were concentrated under vacuum and the crude material was purified by Prep HPLC (Method E) followed by lyophilization to give the title compound as an off white solid (0.020 g, Yield: 7.1%).

¹H NMR (DMSO-d6, 400 MHz): δ ppm 0.7-1.0 (m, 2H), 1.15-1.23 (m, 4H), 1.58-1.61 (m, 7H), 1.92 (s, 3H), 2.19-2.20 (m, 3H), 2.33-2.41 (m, 3H), 3.07-3.08 (m, 1H), 3.12-3.13 (m, 1H), 3.63-3.64 (m, 2H), 4.52 (bs, 2H), 4.71-4.75 (m, 2H), 6.24 (s, 1H), 6.30-6.45 (m, 1H), 6.63-6.76 (m, 1H), 7.03-7.17 (m, 1H), 7.23-7.30 (m, 1H), 7.67 (d, J=8 Hz, 1H), 9.46 (s, 1H), 9.68 (m, 1H). LCMS (Method A): 1.509 min, MS: ES+563.2 (M+1). Analytical HPLC (Method B): 5.180 min. 98%, 230 nm.

### N-(2-(2-(Cyclohexylmethoxy)-4,6-dihydroxy-3-methylbenzoyl)isoindolin-4-yl)but-2-ynamide: Example 12

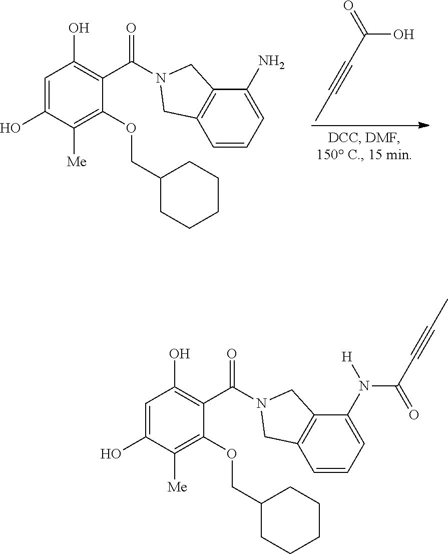

A stirred solution of but-2-ynoic acid (CAS 590-93-2; 0.270 g, 3.21 mmol, 1 eq.), (4-aminoisoindolin-2-yl)(2-(cyclohexylmethoxy)-4,6-dihydroxy-3-methylphenyl)methanone (Intermediate 3) (0.130 g, 0.33 mmol, 0.1 eq.) and DCC (0.662 g, 3.21 mmol, 1 eq.) in DMF (0.3 mL) was heated to 150° C. and stirred for 15 min. The reaction mixture was poured into ice cold water (50 mL) and extracted using ethyl acetate (3×50 mL). The combined organic layer was dried over Na₂SO₄ and concentrated under vacuum. The crude material was purified by flash column chromatography yielding the title compound as an off white solid (0.015 g, Yield: 4.8%).

¹H NMR (DMSO-d6, 400 MHz) mixture of rotamers: δ 0.93-0.99 (m, 3H), 1.09-1.15 (m, 2H), 1.56-1.67 (m, 6H), 1.92 (2 singlets, 3H), 2.00 and 2.07 (2 singlets, 3H), 3.47-3.51 (m, 1H), 3.63-3.65 (m, 1H), 4.50-4.52 (m, 2H), 4.68-4.73 (m, 2H), 6.24-6.26 (2 singlets, 1H), 7.21-7.29 (m, 2H), 7.40-7.44 (m, 1H), 9.40 (s, 1H), 9.47 (m, 1H), 10.25-10.37 (2 singlets, 1H). LCMS (Method A): 2.002 min, 100%, 254 nm MS: ES+463.1. Analytical HPLC Method B: 5.187 min, 98.04%, 254 nm

### (E)-N-(2-(2-(Cyclohexylmethoxy)-4,6-dihydroxy-3-methylbenzoyl)isoindolin-4-yl)but-2-enamide: Example 13

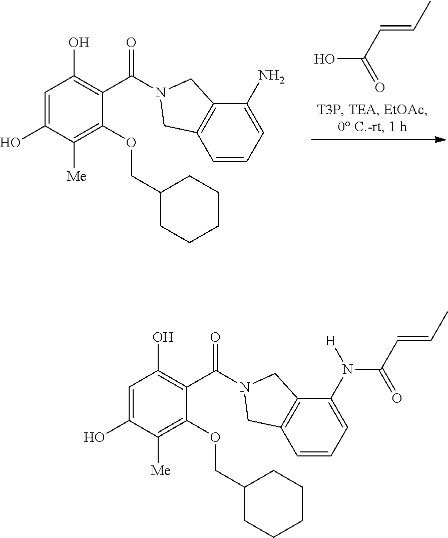

A stirred solution of (E)-but-2-enoic acid (CAS: 107-93-7, 0.110 g, 1.26 mmol, 1 eq.) in EtOAc (1.1 mL) at 0° C. was treated with molecular sieves (catalytic), T3P (50% in EtOAc) (1.21 g, 3.8 mmol, 3 eq.) and TEA (0.51 g, 5.04 mmol, 4 eq.) and stirred for 5 mins. (4-Aminoisoindolin-2-yl)(2-(cyclohexylmethoxy)-4,6-dihydroxy-3-methylphenyl)methanone (Intermediate 3) (0.050 g, 0.13 mmol, 0.1 eq.) was added at 0° C. and the reaction mixture stirred at room temperature for 1 h. The reaction mixture was poured into ice cold water (50 mL), extracted using ethyl acetate (3×50 mL) and the combined organic layer dried over Na₂SO₄ and concentrated under vacuum. The obtained crude material was purified by flash column chromatography followed by prep HPLC purification (Method E) yielding the title compound as a white solid (0.020 g, Yield: 3.3%).

¹H NMR (DMSO-d6, 400 MHz): δ ppm 0.91-1.24 (m, 5H), 1.5-1.68 (m, 6H), 1.82-1.89 (m, 3H), 1.92 (s, 3H), 3.44-3.49 (m, 1H), 3.63-3.66 (m, 1H), 4.51 (s, 2H), 4.70-4.79 (m, 2H), 6.12-6.31 (m, 2H), 6.71-6.85 (m, 1H), 7.02-7.16 (m, 1H), 7.22-7.30 (m, 1H), 7.62-7.67 (m, 1H), 9.39-9.56 (m, 3H). LCMS (Method A): 1.971 min, MS: ES+465.0 (M+1). Analytical HPLC (Method B): 8.167 min, 95%.

| Example | | | |
|---|---|---|---|
| No. | Structure | Name | Data |
| 14 | 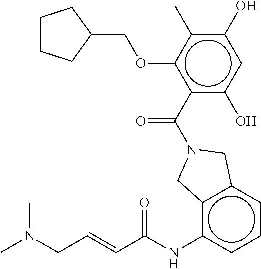 | (E)-N-[2-[2- (Cyclopentylmetho- xy)-4,6-dihydroxy- 3-methyl- benzoyl]isoindolin- 4-yl]-4- (dimethylamino)but- 2-enamide | Prepared in 35% yield according to the Method of Example 2 (DCC/DMF) using (E)-4-(dimethylamino)but-2-enoic acid. HCl and (4-aminoisoindolin-2-yl)(2- (cyclopentylmethoxy)-4,6-dihydroxy-3- methylphenyl)methanone (Intermediate 9). ¹H NMR (DMSO-d6, 400 MHz): δ ppm 1.26-1.29 (m, 2H), 1.43-1.49 (m, 4H), 1.56- 1.65 (m, 2H), 1.92 (s, 3H), 2.14 (s, 3H), 2.19 (s, 3H), 3.00-3.07 (m, 2H), 3.60- 3.70 (m, 2H), 4.47-4.56 (m, 2H), 4.66- 4.79 (m, 2H), 6.29-6.50 (m, 2H), 6.64- 6.79 (m, 1H), 7.02-7.29 (m, 2H), 7.69 (d, J = 7.6 Hz, 1H), 9.60-9.78 (m, 2H). LCMS (Method A): 1.423 min, MS: ES+ 493.98 (M + 1) 100%. |
| 15 | 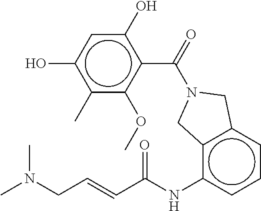 | (E)-N-[2-(4,6- Dihydroxy-2- methoxy-3-methyl- benzoyl)isoindolin- 4-yl]-4- (dimethylamino)but- 2-enamide | Prepared according to the Method of Example 3 (T3P/DEA) using (E)-4- (dimethylamino)but-2-enoic acid. HCl and (4-aminoisoindolin-2-yl)(4,6-dihydroxy-2- methoxy-3-methylphenyl)methanone (Intermediate 10). ¹H NMR (DMSO-d6, 400 MHz): δ ppm 1.92 (s, 3H), 2.14 (s, 3H), 2.19 (s, 3H), 3.00-3.08 (m, 3H), 3.61 (s, 2H), 4.51-4.57 (m, 2H), 4.73-4.76 (m, 2H), 6.33-6.45 (m, 2H), 6.64-6.79 (m, 1H), 7.22-7.30 (m, 1H), 7.64-7.71 (m, 1H), 9.55 (s, 1H), 9.74 (s, 1H). LCMS (Method A): 1.063 min, MS: ES+ 425.97 (M + 1) |
| 16 | 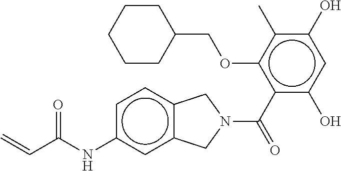 | N-[2-[2- (Cyclohexylmethoxy)- 4,6-dihydroxy-3- methyl- benozyl]isoindolin- 5-yl]prop-2- enamide | Prepared in 17% yield according to the Method of Example 1 using acrylic anhydride and (5-aminoisoindolin-2-yl)(2- (cyclohexylmethoxy)-4,6-dihydroxy-3- methylphenyl)methanone (Intermediate 11). ¹H NMR (DMSO-d6, 400 MHz): δ ppm 0.99-1.23 (m, 5H), 1.57-1.76 (m, 6H), 1.92 (s, 3H), 3.47-3.51 (m, 1H), 3.62-3.65 (m, 1H), 4.45-4.50 (m, 2H), 4.66-4.72 (m, 2H), 5.75-5.77 (m, 1H), 6.22-6.28 (m, 2H), 6.39-6.48 (m, 1H), 7.20-7.34 (m, |
| | | | 1H), 7.46-7.80 (m, 2H), 9.77 (s, 1H), 9.37- |
| | | | 9.47 (m, 1H), 10.19-10.20 (m, 1H). LCMS |
| | | | (Method A): 1.883 min, MS: ES+ 451 |
| | | | (M + 1), 100%. |
| 17 | 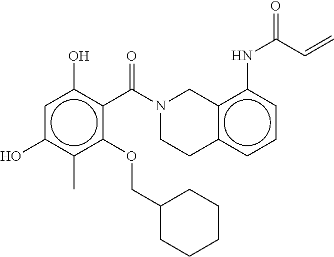 | N-[2-[2- (Cyclohexylmethoxy)- 4,6-dihydroxy-3- methyl-benzoyl]- 3,4-dihydro-1H- isoqinolin-8- yl]prop-2-enamide | Prepared in 28% yield according to the method of Example 1 using acrylic anhydride and (8-amino-3,4- dihydroisoquinolin-2(1H)-yl)(2- (cyclohexylmethoxy)-4,6-dihydroxy-3- methylphenyl)methanone (Intermediate 12). ¹H NMR (DMSO-d6, 400 MHz, 343K): δ ppm 0.91-1.09 (m, 2H), 1.11-1.26 (m, 3H), 1.26-1.70 (m, 6H), 1.92 (s, 3H), 2.85- 2.90 (m, 2H), 3.48-3.57 (m, 4H), 4.20- 4.65 (m, 4H), 5.72-5.75 (m, 1H), 6.20- 6.27 (m, 2H), 6.53-6.55 (m, 1H), 7.02- 7.04 (m, 1H), 7.17 (t, J = 7.6 Hz, 1H), 7.22- 7.26 (m, 1H), 9.03-9.13 (m, 2H), 9.50 (bs, 1H). LCMS (Method A): 1.912 min, MS ES+: 465.02 (M + 1), 99.6%. |
| 18 | 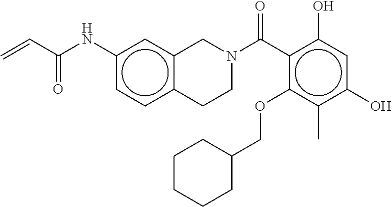 | N-[2-[2- (Cyclohexylmethoxy)- 4,6-dihydroxy-3- methyl-benzyl]- 3,4-dihydro-1H- isoqinolin-7- yl]prop-2-enamide | Prepared in 31% yield according the method of Example 1 using acrylic anhydride and 7-amino-3,4-dihydro- isoquinolin-2(1H)-yl)(2- (cyclohexylmethoxy)-4,6-dihydroxy-3- methylphenyl)methanone (Intermediate 13). ¹H NMR (DMSO-d6, 400 MHz, 344K): δ pm 0.89-0.90 (m, 2H), 1.08-1.11 (m, 3H), 1.56-1.58 (m, 6H), 1.93 (s, 3H), 2.75- 2.77 (m, 2H), 3.43-3.58 (m, 4H), 4.28- 4.36 (m, 1H), 4.68-4.82 (m, 2H), 5.71 (d, J = 12 Hz, 1H), 6.22-6.26 (m, 2H), 6.39- 6.41 (m, 1H), 7.09 (d, J = 7.6 Hz, 1H), 7.42- |
| | | | 7.50 (m, 2H), 9.02 (bs, 1H), 9.16 (bs, 1H), |
| | | | 9.87 (bs, 1H). LCMS (Method A): 1.895 |
| | | | min, MS: ES+ 465.02 (M + 1), 99.4%. |
| 19 | 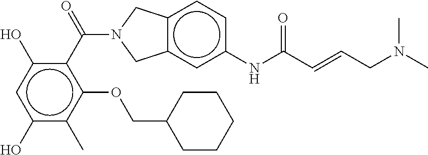 | (E)-N-[2-[2- (Cyclohexylmethoxy)- 4,6-dihydroxy-3- methyl- benzoyl]isoindolin- 5-yl]-4- (dimethylamino)but- 2-enamide | Prepared in 7% yield according to the method of Example 1 using (E)-4- (dimethylamino)but-2-enoic acid. HCl and (5-aminoisoindolin-2-yl)(2- (cyclohexylmethoxy)-4,6-dihydroxy-3- methylphenyl)methanone (Intermediate 11). ¹H NMR (DMSO-d6, 400 MHz): δ ppm 0.86-0.95 (m, 2H), 1.05-1.08 (m, 3H), 1.54-1.58 (m, 6H), 1.90 (d, J = 1.2 Hz, 3H), 2.15 (s, 6H), 3.02-3.04 (m, 2H), 3.48-3.49 |
| | | | (m, 1H), 3.58-3.59 (m, 1H), 4.44-4.47 (m, |
| | | | 2H), 4.65-4.70 (m, 2H), 6.21-6.27 (m, |
| | | | 2H), 6.67-6.76 (m, 1H), 7.19-7.32 (m, |
| | | | 1H), 7.45 (d, J = 8 Hz, 0.5H), 7.53-7.60 (m, |
| | | | 1H), 7.73 (s, 0.5H). LCMS (Method A): |
| | | | 1.486 min, MS: ES+ 508 (M + 1). |
| 20 | 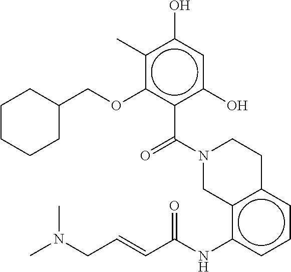 | (E)-N-[2-[2- (Cyclohexylmethoxy)- 4,6-dihydroxy-3- methyl-benzoyl]- 3,4-dihydro-1H- isoquinolin-8-yl]-4- (dimethylamino)but- 2-enamide | Prepared in 20% yield according to the method of Example 2 using (E)-4- (Dimethylamino)but-2-enoic acid hydrochloride and (8-amino-3,4- dihydroisoquinolin-2(1H)-yl)(2- (cyclohexylmethoxy)-4,6-dihydroxy-3- methylphenyl)methanone (Intermediate 12). ¹H NMR (DMSO-d6, 400 MHz, 346K): δ ppm 0.88-0.91 (m, 2H), 1.09-1.15 (m, 3H), 1.57-1.59 (m, 6H), 1.92 (s, 3H), 2.22 (s, 6H), 2.82-2.84 (s, 2H), 3.08-3.15 (m, 2H), 4.30-4.63 (m, 2H), 6.22 (s, 1H), 6.38- 6.40 (m, 1H), 6.73-6.74 (m, 1H), 7.00- 7.02 (m, 1H), 7.16 (t, J = 7.6 Hz, 1H), 7.27 (bs, 1H), 8.40 (s, 2H), 9.39 (bs, 1H), 4 aliphatic protons hidden under the water peak. LCMS (Method A): 1.534 min, MS |
| | | | ES+: 622.03 (M + 1). 100%. |
| 21 | 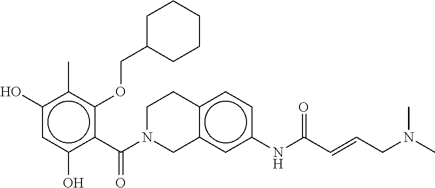 | (E)-N-[2-[2- (Cyclohexylmethoxy)- 4,6-dihydroxy-3- methyl-benzyl[- 3,4-dihydro-1H- isoquinolin-7-yl]-4- (dimethylamino)but- 2-enamide | Prepared in 21% yield according to the method of Example 2 using (E)-4- (dimethylamino)but-2-enoic acid hydrochloride and 7-amino-3,4-dihydro- isoquinolin-2(1H)-yl)(2- (cyclohexylmethoxy)-4,6-dihydroxy-3- methylphenyl)methanone (Intermediate 13). ¹H NMR (DMSO-d6, 400 MHz, 347K): δ ppm 0.88-0.91 (m, 2H), 1.09-1.14 (m, 3H), 1.56-1.58 (m, 6H), 1.93 (s, 3H), 2.20 (s, 6H), 2.75-2.77 (m, 2H), 3.06 (d, J = 5.6 Hz, 2H), 3.39-3.51 (m, 3H), 4.32-4.36 (m, |
| | | | 1H), 4.65-4.68 (m, 2H), 6.22-6.27 (m, |
| | | | 2H), 6.71-6.75 (m, 1H), 7.08 (d, J = 8 Hz, |
| | | | 1H), 7.30-7.41 (m, 2H), 9.78 (bs, 2H). |
| | | | Formate salt. LCMS (Method A): 1.472 min, |
| | | | MS: ES+ 522.1 (M + 1), 96.7%. |
| 22 | 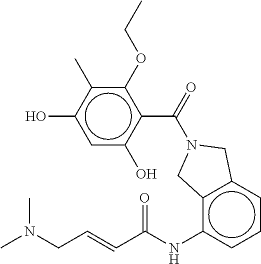 | (E)-4- (Dimethylamino)-N- [2-(2-ethoxy-4,6- dihydroxy-3- methyl- benzoyl)isoindolin- 4-yl]but-2-enamide | Prepared in 10% yield according to the method of Example 2 using (E)-4- (dimethylamino)but-2-enoic acid hydrochloride and (4-aminoisoindolin-2- yl)(2-ethoxy-4,6-dihydroxy-3- methylphenyl)methanone (Intermediate 14). ¹H NMR (DMSO-d6, 400 MHz): δ ppm 1.16-1.19 (t, 3H), 1.92 (s, 3H), 2.15 (s, 3H), 2.20 (s, 3H), 3.02-3.12 (m, 2H), 3.77-3.87 (m, 2H), 4.45-4.58 (m, 2H), 4.73-4.76 (m, 2H), 6.25 (s, 1H), 6.26-6.45 (m, 1H), 6.64- 6.79 (m, 1H), 7.03-7.16 (m, 1H), 7.23- 7.30 (m, 1H), 7.66-7.71 (m, 1H), 8.20 (s, 1H, formate salt), 9.52 (bs, 2H), 9.69 (s, 1H). LCMS (Method A): 1.135 min, MS: ES+ 440 (M + 1), 97.7%. |
| 23 | 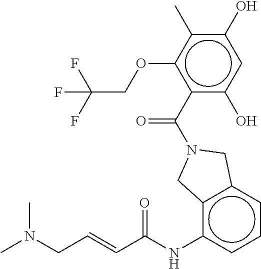 | (E)-N-[2-[4,6- Dihydroxy-3- methyl-2-(2,2,2- trifluoroethoxy)ben- zoyl]isoindolin-4- yl]-4- (dimethylamino)but- 2-enamide | Prepared in 24% yield using the method of Example 2 using (E)-4-(dimethylamino)but- 2-enoic acid hydrochloride and (4- aminoisoindolin-2-yl)(4,6-dihydroxy-3- methyl-2-(2,2,2- trifluoroethoxy)phenyl)methanone (Intermediate 15). ¹H NMR (DMSO-d6, 400 MHz): δ ppm 1.95 (s, 3H), 2.16 (s, 3H), 2.21 (s, 3H), 3.03-3.10 (dd, J = 5.2 Hz, 2H), 4.35- 4.42 (m, 1H), 4.47-4.57 (m, 2H), 4.59- 4.63 (m, 1H), 4.75 (d, J = 12 Hz, 2H), 6.28- 6.45 (m, 1H), 6.35 (s, 1H), 6.65-6.78 (m, 1H), 7.03-7.16 (m, 1H), 7.24-7.31 (m, 1H), 7.69 (t, J = 8.4 Hz, 2H), 8.15 (s, 1H, formate salt), 9.50 (s, 1H), 9.66-9.71 (m, 2H). LCMS (Method A): 1.301 min, MS ES+: 493.9 (M + 1), 100%. |
| 24 | 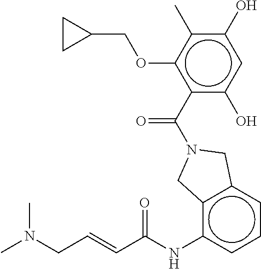 | (E)-N-[2-[2- (cyclopropylmethoxy)- 4,6-dihydroxy-3- methyl- benozyl]isoindolin- 4-yl]-4- (dimethylamino)but- 2-enamide | Prepared in 11% yield according to the method of Example 2 using (E)-4- (dimethylamino)but-2-enoic acid hydrochloride and (4-aminoisoindolin-2- yl)(2-(cyclopropylmethoxy)-4,6-dihydroxy- 3-methylphenyl)methanone (Intermediate 16). ¹H NMR (DMSO-d6, 400 MHz): δ ppm 0.20-0.22 (m, 2H), 0.43-0.46 (m, 2H), 1.07- 1.11 (m, 1H), 1.94 (d, J = 0.8 Hz, 3H), 2.16- 2.21 (m, 6H), 3.02-3.17 (m, 3H), 3.51- 3.76 (m, 2H), 4.51-4.57 (m, 2H), 4.73- 4.76 (m, 2H), 6.40 (s, 1H), 6.42-6.46 (m, 2H), 6.65-6.68 (m, 1H), 7.03-7.17 (m, 1H), 7.24-7.31 (m, 1H), 9.52 (s, 1H), 9.68 (s, 1H). Formic salt. LCMS (Method A): 1.221 min, MS ES+: 466 (M + 1). |
| 25 | 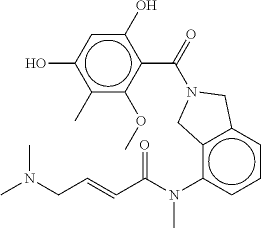 | (E)-N-[2-(4,6- Dihydroxy-2- methoxy-3-methyl- benozyl)isoindolin- 4-yl]-4- (dimethylamino)-N- methyl-but-2- enamide | Prepared in 10% yield according to the method of Example 2 using (E)-4- (dimethylamino)but-2-enoic acid hydrochloride and (4,6-dihydroxy-2- methoxy-3-methylphenyl)(4- (methylamino)isoindolin-2-yl)methanone (Intermediate 17). ¹H NMR (D₂O exchange) (DMSO-d6, 400 MHz): δ ppm 1.89 (s, 3H), 2.13 (s, 6H), 2.96-3.06 (m, 3H), 3.19 (s, 2H), 3.58 (s, 3H), 4.50-4.83 (m, 4H), 5.79- 5.83 (m, 1H), 6.24 (s, 1H), 6.59-6.69 (m, 1H), 7.22 (d, J = 7.6 Hz, 1H), 7.31 (d, J = 7.6 Hz, 1H), 7.38-7.44 (m, 1H), 8.22 (1H, formate salt). LCMS (Method A): 1.087 |
| | | | min, MS ES+: 439.97 (M + 1), 100%. |
| 26 | 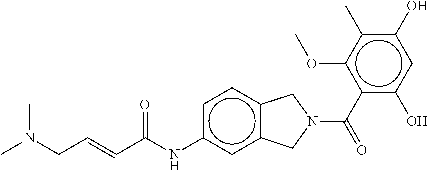 | (E)-N-[2-(4,6- Dihydroxy-2- methoxy-3-methyl- benzoyl)isoindolin- 5-yl]-4- (dimethylamino)but- 2-enamide | Prepared in 6% yield according to the method of Example 2 using (E)-4- (dimethylamino)but-2-enoic acid hydrochloride and (5-aminoisoindolin-2- yl)(4,6-dihydroxy-2-methoxy-3- methylphenyl)methanone (Intermediate 18). ¹H NMR (DMSO-d6, 400 MHz): δ ppm 1.92 (s, 3H), 2.18 (s, 3H), 2.19 (s, 3H), 3.05- 3.08 (m, 2H), 3.58 (s, 3H), 4.42-4.52 (m, 2H), 4.72 (d, J = 13.2 Hz, 2H), 6.25-6.29 (m, 2H), 6.67-6.75 (m, 1H), 7.19-7.30 (dd, |
| | | | J = 8 Hz, 1H), 7.46 (d, J = 8.4 Hz, 1H), 7.55- |
| | | | 7.57 (m, 1H), 7.59 (s, 1H), 8.19 (s, 1H, |
| | | | Formate salt), 9.46 (bs, 2H), 10.11 (d, J = |
| | | | 8.4 Hz, 1H). LCMS (Method A): 0.987 min, |
| | | | MS ES+: 426 (M + 1), 99.6%. |
| 27 | 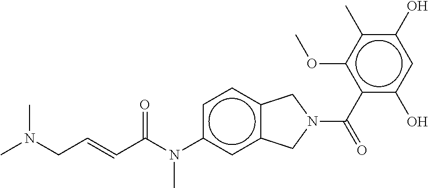 | (E)-N-[2-(4,6- dihydroxy-2- methoxy-3-methyl- benzoyl)isoindolin- 5-yl]-4- (dimethylamino)-N- methyl-but-2- enamide | Prepared in 5% yield according to the method of Example 2 using (E)-4- (dimethylamino)but-2-enoic acid hydrochloride and (4,6-dihydroxy-2- methoxy-3-methylphenyl)(5- (methylamino)isoindolin-2-yl)methanone (Intermediate 19). ¹H NMR (DMSO-d6, 400 MHz): δ ppm 1.92 (s, 3H), 2.06 (s, 3H), 2.07 (s, 3H), 2.89-2.90 (m, 2H), 3.19-3.22 (m, 4H), 3.60 (s, 3H), 4.47-4.54 (m, 2H), 4.76- 4.78 (m, 1H), 5.85-5.87 (m, br, 1H), 6.27 (s, 1H), 6.59-6.65 (m, br, 1H), 7.15-7.21 |
| | | | (m, 1H), 7.31-7.34 (m, 1H), 7.43-7.45 (m, |
| | | | 1H), 8.18, s, 1H, Formate salt) 9.52 (bs, |
| | | | 2H). LCMS (Method A): 1.060 min, MS |
| | | | ES+: 440.17 (M + 1), 99.2%. |
| 28 | 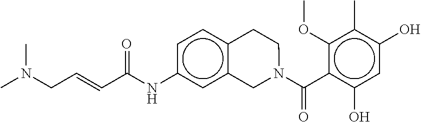 | (E)-N-[2-(4,6- dihydroxy-2- methoxy-3-methyl- benzoyl)-3,4- dihydro-1H- isoquinolin-7-yl]-4- (dimethylamino)but- 2-enamide | Prepared in 33% yield according to the method of Example 2 using (E)-4- (dimethylamino)but-2-enoic acid hydrochloride and (7-amino-3,4- dihydroisoquinolin-2(1H)-yl)(4,6-dihydroxy- 2-methoxy-3-methylphenyl)methanone (Intermediate 20). ¹H NMR (DMSO-d6, 400 MHz, 348K): δ ppm 1.94 (s, 3H), 2.20 (s, |
| | | | 6H), 2.75-2.77 (m, 2H), 3.07 (d, J = 5.6 Hz, |
| | | | 2H), 3.58 (s, 5H), 4.60-4.61 (m, 2H), 6.24- |
| | | | 6.27 (m, 2H), 6.71-6.75 (m, 1H), 7.08 (d, |
| | | | J = 8.4 Hz, 1H), 7.42 (bs, 2H), 8.20 (s, 1H, |
| | | | Formate salt), 9.06 (s, 2H), 9.78 (s, 1H). |
| | | | LCMS (Method A): 1.087 min. MS ES+: |
| | | | 440.07 (M + 1), 96.8%. |
| 29 | 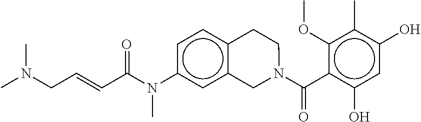 | (E)-N-[2-(4,6- dihydroxy-2- methoxy-3-methyl- benzoyl)-3,4- dihydro-1H- isoquinolin-7-yl]-4- (dimethylamino)-N- methyl-but-2- | Prepared in 20% yield according to the method of Example 2 using (E)-4- (dimethylamino)but-2-enoic acid hydrochloride and (4,6-dihydroxy-2- methoxy-3-methylphenyl)(7- (methylamino)-3,4-dihydroisoquinolin- 2(1H)-yl)methanone (Intermediate 21). ¹H NMR (DMSO-d6, 400 MHz, 348K): δ ppm |
| | | enamide | 1.94 (s, 3H), 2.66 (s, 3H), 2.81-2.86 (m, |
| | | | 2H), 3.26 (bs, 3H), 3.54-3.60 (m, 9H), 3.73- |
| | | | 3.74 (m, 2H), 4.71-4.72 (m, 1H), 6.20- |
| | | | 6.28 (m, 2H), 6.63-6.69 (m, 1H), 7.08- |
| | | | 7.25 (m, 3H), 9.04 (bs, 1H), 10.63 (bs, 1H). |
| | | | LCMS (Method A): 1.092 min, MS ES+: |
| | | | 454.17 (M + 1), 100%. |
| 30 | 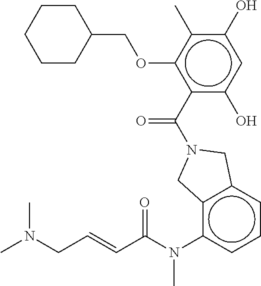 | (E)-N-[2-[2- (Cyclohexylmethoxy)- 4,6-dihydroxy-3- methyl- benzoyl]isoindolin- 4-yl]-4- (dimethylamino)-N- methyl-but-2- enamide | Prepared in 2% yield according to the method of Example 2 using (E)-4- (dimethylamino)but-2-enoic acid hydrochloide and (2-(cyclohexylmethoxy)- 4,6-dihydroxy-3-methylphenyl)(4- (methylamino)isoindolin-2-yl)methanone (Intermediate 22). ¹H NMR (DMSO-d6, 400 MHz): δ ppm 0.86-1.00 (m, 2H), 1.10- 1.24 (m, 3H), 1.45-1.70 (m, 6H), 1.89- 1.92 (m, 3H), 2.62-2.67 (m, 4H), 2.92 (s, 1H), 3.10 (s, 1H), 3.21 (s, br, 2H), 3.63- 3.65 (m, 1H), 3.73-3.74 (m, 1H), 4.25- 4.66 (m, 3H), 4.80-4.81 (m, 1H), 5.97- 6.90 (m, 1H), 6.27-6.28 (m, 1H), 6.64- 6.76 (m, 1H), 7.19-7.3 (m, 2H), 7.36-7.45 (m, 1H), 7.46-7.47 (m, 1H), 9.43-9.47 (m, 1H), 9.54 (s, 1H)., 10.35-10.36 (m, 1H). LCMS (Method A): 1.536 min, MS: ES+ |
| | | | 522.1 (M + 1), 100%. |
| 31 | 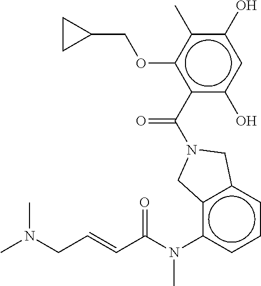 | (E)-N-[2-[2- (Cyclopropylmetho- xy)-4,6-dihydroxy- 3-methyl- benzoyl]isoindolin- 4-yl]-4- (dimethylamino)-N- methyl-but-2- enamide | Prepared in 1.3% yield according to the method of Example 2 using (E)-4- (dimethylamino)but-2-enoic acid hydrochloride and (2- (cyclopropylmethoxy)-4,6-dihydroxy-3- methylphenyl)(4-(methylamino)isoindolin- 2-yl)methanone (Intermediate 23). ¹H NMR (DMSO-d6, 400 MHz, D₂O exchange): δ ppm 0.14-0.37 (m, 2H), 0.41-0.57 (m, 2H), 1.04-1.19 (m, 1H), 1.86 and 1.88 (2 singlets, 3H), 2.51 (s, 6H), 3.07 and 3.21 (2 singlets, 3H), 3.63-3.65 (m, 2H), 3.72- 3.74 (m, 2H), 4.34-4.96 (m, 4H), 5.98- 6.02 (m, 1H), 6.21 (s, 1H), 6.45-6.55 (m, 1H), 7.19-7.46 (m, 3H). LCMS (Method A): 1.265 min, MS ES+: 480.08 (M + 1), 97.5%. |
| 32 | 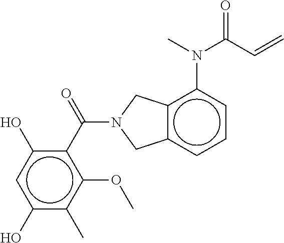 | N-[2-(4,6- dihydroxy-2- methoxy-3-methyl- benzoyl)isoindolin- 4-yl]-N-methyl- prop-2-enamide | Prepared in 8.8% yield according to the method of Example 1 using acrylic anhydride and (4,6-dihydroxy-2-methoxy- 3-methylphenyl)(4- (methylamino)(isoindolin-2-yl)methanone (Intermediate 17). ¹H NMR (DMSO-d6, 400 MHz): δ ppm 1.90 and 1.91 (2 singlets, 3H), 3.09-3.22 (2 singlets, 3H), 3.60 (s, 2H), 4.51-4.83 (m, 4H), 5.60 (d, J = 12.8 Hz, 1H), 5.85-6.08 (m, 1H), 6.20-6.32 (m, 2H), 7.22-7.44 (m, 3H), 9.89 (s, 2H), LCMS (Method A): 1.357 min, MS ES+: 439.97 (M + 1), 97%. |
| 33 | 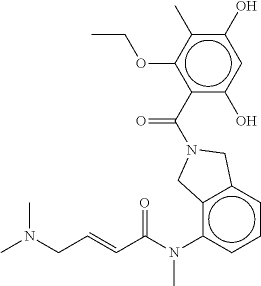 | (E)-4- (Dimethylamino)-N- (2-(2-ethoxy-4,6- dihydroxy-3- methylbenozyl)isoin- dolin-4-yl)-N- methylbut-2- enamide | Prepared in 29% yield according to the method of Example 2 using (E)-4- (dimethylamino)but-2-enoic acid hydrohcloride and (2-Ethoxy-4,6- dihydroxy-3-methylphenyl)(4- (methylamino)isoindolin-2-yl)methanone (Intermediate 24). ¹H NMR (DMSO-d6, 400 MHz): δ ppm 1.13-1.17 (s, br, 3H), 1.90- 1.92 (s, br, 3H), 2.07 and 2.11 (2 singlets, 6H), 2.90-2.98 (m, 2H), 3.07 and 3.15 (2 singlets, 3H), 3.75-3.79 (m, 2H), 4.39- 4.82 (m, 4H), 5.73-5.80 (m, 1H), 6.16- 6.24 (m, 1H), 6.68-6.70 (m, 1H), 7.22- 7.44 (m, 3H), 8.21 (s, 1H, formate salt). LCMS (Method A): 1.157 min, MS ES+: 454.00 (M + 1), 98.4%. |

### (E)-4-[2-(4,6-Dihydroxy-2-methoxy-3-methyl-benzoyl)isoindolin-5-yl]oxy-N,N-dimethyl-but-2-enamide: Example 34

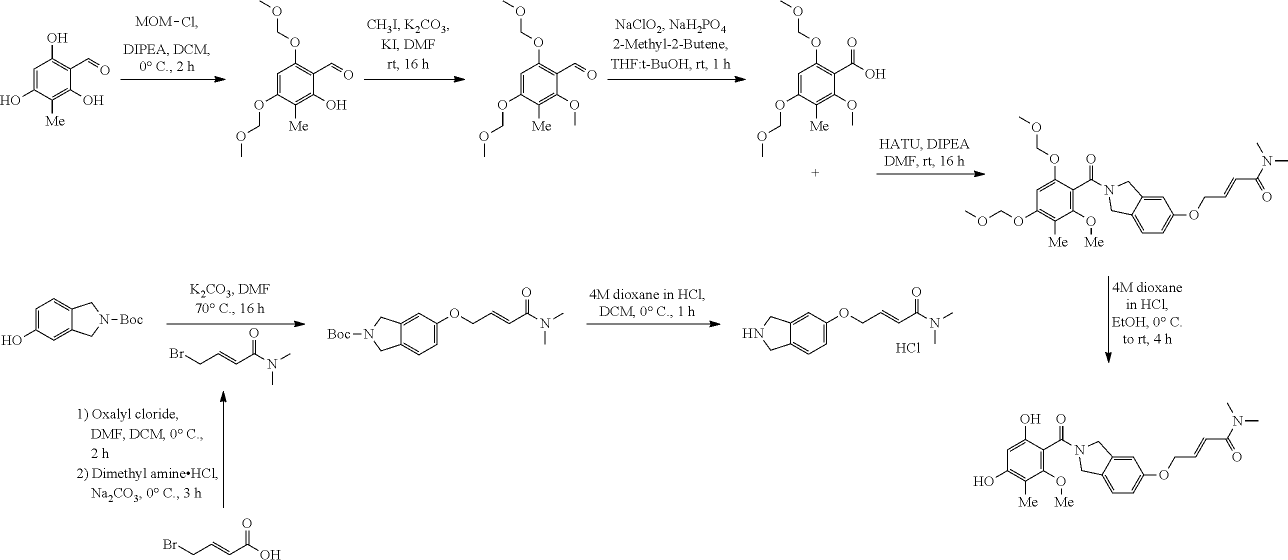

### 2-Hydroxy-4,6-bis(methoxymethoxy)-3-methylbenzaldehyde

A stirred solution of 2,4,6-trihydroxy-3-methylbenzaldehyde (see preparation of Intermediate 6) (5 g, 29.76 mmol, 1 eq.) in DCM (50 mL) at 0° C. was treated with DIPEA (19.19 g, 148 mmol, 5 eq.) and stirred for 5 min. MOM-CI (7.18 g, 89 mmol, 3 eq.) was added at 0° C. and the reaction mixture stirred at room temperature for 30 min. The reaction mixture was poured into water (300 mL) and extracted into DCM (3×100 mL). The combined organic layer was dried over Na₂SO₄ and concentrated under vacuum. Crude material was purified by flash chromatography (product eluted in 15% in ethyl acetate in Hexane) yielding the title compound as a yellow solid (4.4 g, yield: Quantitative).

¹H NMR (DMSO-d6, 400 MHz): δ 1.95 (s, 3H), 3.41 (s, 3H), 3.44 (s, 3H), 5.32 (d, J=7.2 Hz, 4H), 6.44 (s, 1H), 10.12 (s, 1H), 12.39 (s, 1H). LCMS (Method A): 1.884 min, 1.991 min, MS: ES+256.88 (M+1)

### 2-Methoxy-4,6-bis(methoxymethoxy)-3-methylbenzaldehyde

A stirred solution of 2-hydroxy-4,6-bis(methoxymethoxy)-3-methylbenzaldehyde (4.4 g, 17.18 mmol, 1 eq.) in DMF (44 mL) at room temperature was treated with K₂CO₃ (11.85 g, 85.93 mmol, 5 eq.) and KI (0.85 g, 5.15 mmol, 0.3 eq.) and stirred for 5 min. CH₃l (3.63 g, 25.78 mmol, 1.5 eq.) was added and the reaction mixture was stirred at room temperature for 16 h. The reaction mixture was poured into water (300 mL) and extracted with EtOAc (3×100 mL) and the organic layer washed with brine solution (100 mL). The combined organic layer was dried over Na₂SO₄, filtered, and concentrated under vacuum yielding the title compound as a yellow sticky solid (4.7 g, Yield: 98.7%); LCMS (Method A): 1.645 min, 1.691 min, 1.718 min, MS: ES+270.79 (M+1). Crude material was used in the next step without purification.

### 2-Methoxy-4,6-bis(methoxymethoxy)-3-methylbenzoic acid

Carried out in two batches in parallel. A stirred solution 2-methoxy-4,6-bis(methoxymethoxy)-3-methylbenzaldehyde (0.6 g, 2.22 mmol, 1 eq.) in THF: t-BuOH (1:3) (6 mL) at room temperature was treated with saturated aqueous NaClO₂ (0.699 g, 7.70 mmol, 3.5 eq.) and NaH₂PO₄ (1.70 g, 14.20 mmol, 6.4 eq.). 2-Methyl-2-butene (2.18 g, 31.11 mmol, 14 eq.) was added at room temperature and the reaction mixture stirred for 30 mins, poured into saturated solution of NH₄Cl (50 mL) and extracted in DCM (3×80 mL). The combined organic layer was dried over Na₂SO₄, filtered, and concentrated under vacuum yielding the title compound acid as a yellow liquid (1.25 g, Yield: 97%, 4.37 mmol). Crude material was used in the next step without purification.

### (E)-4-Bromo-N,N-dimethylbut-2-enamide

Carried out in four batches in parallel. A stirred solution of (E)-4-bromo-but-2-enoic acid (0.5 g, 12.12 mmol, 1 eq.) (CAS 13991-36-1) in DCM (10 mL) at 0° C. was treated with DMF (0.4 mL) and stirred for 10 mins. Oxalyl chloride (0.576 g, 4.54 mmol, 1.5 eq.) was added to reaction mixture at 0° C. and stirred for 2 h. Dimethylamine hydrochloride (0.37 g, 4.64 mmol, 1.5 eq.) and Na₂CO₃ (0.95 g, 9.09 mmol, 3.0 eq.) were added at 0° C. and the reaction mixture stirred for 2 h. The reaction mixture was diluted with water (200 mL) and extracted with DCM (2×200 mL). The combined organic layer was dried over anhydrous Na₂SO₄ filtered and concentrated under vacuum yielding (E)-4-bromo-N,N-dimethylbut-2-enamide as a purple liquid (2.1 g, Yield: 90.6%).

¹H NMR (DMSO-d6, 400 MHz): δ 3.08 (s, 6H), 4.06-4.08 (m, 2H), 6.50-6.53 (m, 1H), 6.92-6.99 (m, 1H). LCMS (Method A): 1.032 min, MS: ES+193.73 (M+1).

### t-Butyl (E)-5-((4-(dimethylamino)-4-oxobut-2-en-1-yl)oxy)isoindoline-2-carboxylate

A stirred solution of t-butyl-5-hydroxyisoindoline-2-carboxylate (CAS 226070-47-9) (2 g, 8.5 mmol, 1 eq.) in DMF (20 mL) at 0° C. was treated with K₂CO₃ (3.52 g, 25.50 mmol, 3.0 eq.) and stirred for 10 mins. (E)-4-bromo-N,N-dimethylbut-2-enamide (2.42 g, 12.75 mmol, 1.5 eq.) was added at 0° C. and the resulting reaction mixture heated to 70° C. and stirred for 16 h. The reaction mixture was cooled to room temperature, poured over ice cold water (200 mL) and extracted with EtOAc (3×200 mL). The combined organic layer was dried over Na₂SO₄, filtered, and concentrated under vacuum. The obtained crude material was purified by flash chromatography (eluting with 6% MeOH in DCM) yielding the title compound as a brown liquid (0.6 g, Yield: 20.4%). LCMS (Method A): 1.890 min, 1.943 min, MS: ES+347 (M+1), 247 (M-100).

### (E)-4-(Isoindolin-4-yloxy)-N,N-dimethylbut-2-enamide.HCl

A stirred solution of t-butyl (E)-4-((4-(dimethylamino)-4-oxobut-2-en-1-yl)oxy)isoindoline-2-carboxylate (0.7 g, 2.02 mmol, 1 eq.) in DCM (7 mL) at 0° C. was treated with 4M HCl in dioxane (7 mL) added dropwise. The resulting reaction mixture was stirred at room temperature for 2 h. The reaction mixture was concentrated under vacuum to generate crude material which was further triturated with diethyl ether (2×5 mL) followed by high vacuum yielding the title compound (0.5 g, Yield: Quantitative). LCMS (Method A): 0.796 min, MS: ES+246.88 (M+1).

### (E)-4-((2-(2-Methoxy-4,6-bis(methoxymethoxy)-3-methylbenzoyl)isoindolin-5-yl)oxy)-N,N-dimethylbut-2-enamide

A stirred solution of 2-methoxy-4,6-bis(methoxymethoxy)-3-methylbenzoic acid (0.60 g, 2.11 mmol, 1 eq.) in DMF (6 mL) at 0° C. was treated with DIPEA (0.72 mL, 4.23 mmol, 2.0 eq.) and HATU (1.20 g, 3.17 mmol, 1.5 eq.) and stirred for 30 minutes. (E)-4-(isoindolin-4-yloxy)-N,N-dimethylbut-2-enamide HCl (0.62 g, 2.53 mmol, 1.2 eq.) was added to the reaction mixture at 0° C. The resulting reaction mixture was stirred at room temperature for 16 h and then poured into ice cold water (100 mL) and extracted with EtOAc (3×100 mL). The combined organic layer was dried over Na₂SO₄, filtered, and concentrated under vacuum. The crude material was purified by flash chromatography (product eluted at 4% MeOH in DCM) yielding the title compound as an orange liquid (0.47 g, Yield:36.8%). LCMS (Method A): 1.702 min, 1.759 min, 1.790 min, MS: ES+515 (M+1).

### (E)-4-[2-(4,6-dihydroxy-2-methoxy-3-methyl-benzoyl)isoindolin-5-yl]oxy-N,N-dimethyl-but-2-enamide

A stirred solution of (E)-4-((2-(2-methoxy-4,6-bis(methoxymethoxy)-3-methylbenzoyl)isoindolin-5-yl)oxy)-N,N-dimethylbut-2-enamide (0.43 g, 0.83 mmol, 1 eq.) in EtOH (4.3 mL) at 0° C. was treated with 4M HCl in dioxane (2.15 mL) added dropwise. The resulting reaction mixture was stirred at room temperature for 4 h and then concentrated under vacuum. Crude material was purified by flash chromatography (product eluted at 6% MeOH in DCM) and HPLC purification yielding the title compound as a white solid (0.02 g, Yield: 9.1%).

¹H NMR (DMSO-d6, 400 MHz): δ 1.92 (s, 3H), 2.87 (d, J=4.8 Hz, 3H), 3.02 (d, J=8.4 Hz, 3H), 3.14 (bs, 2H), 3.60 (s, 3H), 4.43 (bs, 2H), 4.67-4.75 (m, 2H), 6.25 (s, 1H), 6.66-6.73 (m, 2H), 6.90 (t, J=7.6 Hz, 1H), 6.98-7.01 (m, 1H), 7.13-7.29 (m, 1H), 9.42-9.48 (m, 2H). LCMS (Method A): 1.360 min, MS: ES+426 (M+1).

### (E)-4-((2-(4,6-Dihydroxy-2-methoxy-3-methylbenzoyl)isoindolin-4-yl)oxy)-N,N-dimethylbut-2-enamide: Example 35

Prepared using methods described for Example 34 above using of t-butyl 4-hydroxyisoindoline-2-carboxylate (CAS 871013-92-2).

¹H NMR (DMSO-d6, 400 MHz): δ 1.92 (s, 3H), 2.85-3.05 (m, 6H), 3.61 (s, 3H), 4.37-4.54 (m, 2H), 4.70-4.83 (m, 4H), 6.25 (s, 1H), 6.58-6.66 (m, 1H), 6.75 (s, br, 1H), 6.85-6.98 (m, 2H), 7.22-7.30 (m, 1H), 9.44-9.52 (m, 2H), 11.07 (s, 1H). LCMS (Method A): 1.417 min, MS: ES+426.9 (M+1), 97%.

### N-(2-(4,6-Dihydroxy-2-methoxy-3-methylbenzoyl) isoindolin-4-yl)-2-((dimethyl amino)methyl) acrylamide: Example 36

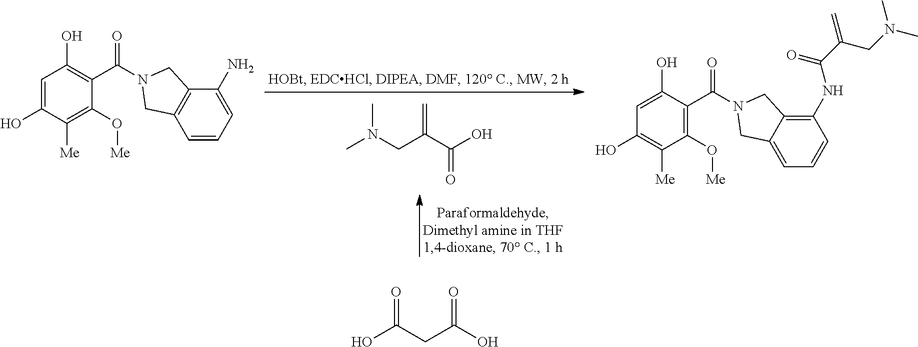

A stirred solution of malonic acid (1.0 g, 9.6 mmol, 1.0 eq.) (CAS:141-82-2) in 1,4-dioxane (10 mL) at room temperature was treated with paraformaldehyde (0.63 g, 20 mmol, 2.3 eq.). Dimethyl amine (2M in THF) (5.0 mL, 10 mmol, 1.05 eq.) was added and the resulting reaction mixture heated to 70° C. and stirred for 1 h. The reaction mixture was concentrated under vacuum yielding 2-((dimethylamino)methyl) acrylic acid (1.05 g, Yield:84%).

¹H NMR (D₂O, 400 MHz): 2.50 (s, 6H), 3.59 (bs, 2H), 5.51 (s, 1H), 6.01 (s, 1H). LCMS (Method B): 0.59 min, MS: ES+130.1 (M+1).

A stirred solution of 2-((dimethylamino)methyl) acrylic acid (0.2 g, 1.548 mmol, 1.0 eq.) in DMF (4 mL) at 0° C. was treated with DIPEA (0.199 g, 1.548 mmol, 1.0 eq.), HOBt (0.20 g, 1.548 mmol, 1.0 eq.) and EDC.HCl (0.59 g, 3.09 mmol, 1.0 eq.); the reaction mixture was stirred for 5 minutes at 0° C., then treated with (4-aminoisoindolin-2-yl)(4,6-dihydroxy-2-methoxy-3-methylphenyl)methanone (Intermediate 10) (0.072 g, 0.23 mmol, 0.15 eq.). The reaction mixture was heated to 120° C. under microwave irradiation for 2 h, then poured into water (50 mL) and extracted with EtOAc (2×100 mL). The combined organic layer washed by brine solution (2×30 mL), dried over Na₂SO₄, filtered, and concentrated under vacuum. The crude material was purified by prep. HPLC purification followed by lyophilisation yielding the title compound (0.016 g, Yield: 8.1%).

¹H NMR (DMSO-d6, 400 MHz, 348K): 1.96 (s, 3H), 2.87 (s, 3H), 2.92 (s, 3H), 3.65 (s, 3H), 3.84 (bs, 1H), 3.94 (bs, 1H), 4.39 (s, 1H), 4.46 (bs, 1H), 4.63 (bs, 1H), 4.71 (bs, 1H), 5.06-5.13 (m, 1H), 5.24-5.36 (m, 1H), 6.28 (s, 1H), 6.44-6.60 (m, 2H), 7.03-7.07 (m, 1H). OH protons not observed. LCMS (Method A): 1.435 min, MS: ES+426.17 (M+1), 99.7%.

### N-(2-(4,6-Dihydroxy-2-methoxy-3-methylbenzoyl)isoindolin-4-yl)-2-(morpholinomethyl)acrylamide: Example 37

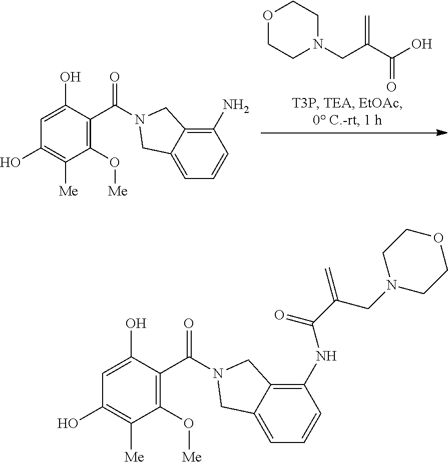 [SMILES: COc1c(C)c(O)cc(O)c1C(=O)N1Cc2cccc(N)c2C1 ; C=C(CN1CCOCC1)C(=O)O ; C=C(CN1CCOCC1)C(=O)Nc1cccc2c1CN(C(=O)c1c(O)cc(O)c(C)c1OC)C2]

A stirred solution of (4-aminoisoindolin-2-yl)(4,6-dihydroxy-2-methoxy-3-methylphenyl)methanone (Intermediate 10) (0.1 g, 0.32 mmol, 1 eq.) in ethyl acetate (1 mL) at 0° C. was treated with molecular sieves (cat.), 2-(morpholinomethyl)acrylic acid (0.11 g, 0.64 mmol, 2 eq.) (CAS: 4432-44-4), T₃P (50% in ethyl acetate) (0.304 g, 0.96 mmol, 3 eq.) and TEA (0.26 g, 2.58 mmol, 8 eq.). The reaction mixture was stirred at room temperature for 1 h, then concentrated under vacuum. Crude material was purified by flash chromatography (product eluted with 7% MeOH in DCM) yielding the title compound as a white solid (0.020 g, Yield: 13%).

¹H NMR (DMSO-d6, 400 MHz, 350K): δ ppm 1.96 (s, 3H), 2.30-2.42 (m, 2H), 2.54-2.55 (m, 2H), 2.68-2.91 (m, 1H), 3.23 (s, 1H), 3.29 (s, br, 2H), 3.37 (s, 1H), 3.65 (s, 3H), 3.80 (s, br, 2H), 4.59 (d, J=9.6 Hz, 2H), 4.83 (bs, 2H), 5.57-5.64 (m, 1H), 6.12-6.18 (m, 1H), 6.25-6.27 (m, 1H), 7.02-7.17 (m, 1H), 7.27-7.33 (m, 1H), 7.91-7.97 (m, 1H), 9.16 (bs, 2H), 10.20-10.46 (m, 1H). LCMS (Method A): 1.054 min, MS: ES+468.1 (M+1), 100%.

### Prep. HPLC Purification Methods

### Prep. HPLC Purification Method—Example 14

Purification was conducted with a Shimadzu LC20AP purification system with a UV detector. Column: XTIMATE C18, (21.2×250)mm, 5 μm; compounds eluted with: Mobile Phase A : 0.05% Formic acid in water, Mobile Phase B: Acetonitrile with a gradient of T=0 min (64% A, 36% B); gradient to T=12.00 min (64% A, 36% B); T=12.01 min (02% A, 98% B) gradient to T=14.00 min (02% A, 98% B); T=14.01 min (64% A, 36% B); gradient to T=16 min (64% A, 36% B); Flow rate=16 ml/min; analysis time 16 min.

### Prep. HPLC Purification Method—Example 15

Purification was conducted with a Waters 2545 purification system with a UV detector. Column: SUNFIRE Prep C18 OBD, (19×250)mm, 5 μm; compounds were eluted with, Mobile Phase A: 0.1% Formic acid in water, Mobile Phase B: Acetonitrile with gradient of T=0 min (91% A, 09% B); gradient to T=18.00 min (86% A, 14% B); T=18.01 min (02% A, 98% B) gradient to T=20.00 min (02% A, 98% B); Flow rate=21 ml/min, T=20.01 min (91% A, 09% B); gradient to T=22 min (91% A, 09% B); Flow rate=18 ml/min; analysis time 22 min.

### Prep. HPLC Purification Method—Example 19

Purification was conducted with a Shimadzu LC20AP purification system with a UV detector. Column: SHIM-PACK GIST C18, (20×250)mm, 5 μm; compounds eluted with, Mobile Phase A: Water, Mobile Phase B: Acetonitrile with a gradient of T=0 min (62% A, 38% B); gradient to T=16.00 min (50% A, 50% B); T=16.01 min (02% A, 98% B) gradient to T=18.00 min (02% A, 98% B); T=18.01 min (62% A, 38% B); gradient to T=21 min (62% A, 38% B); Flow rate=20 ml/min; analysis time 21 min.

### Prep. HPLC Purification Method—Example 20

Purification was conducted with a Waters 2545 purification system with a UV detector. Column: Xbridge Prep, C18,OBD (19×250)mm, 5 μm; compounds eluted with, Mobile Phase A: 0.05% Ammonium Hydroxide in water, Mobile Phase B: Acetonitrile with a gradient of T=0 min (79% A, 21% B); gradient to T=20.00 min (75% A, 25% B); T=20.01 min (02% A, 98% B) gradient to T=22.00 min (02% A, 98% B); T=22.01 min (79% A, 21% B); gradient to T=25 min (79% A, 21% B); Flow rate=18 ml/min; analysis time 25 min.

### Prep. HPLC Purification Method—Example 21

Purification was conducted with a Flash Chromatography-SELEKT system with a UV detector. Column: YMC 120 g C18, 50 μm: compounds eluted with, Mobile Phase A: 0.1% Formic acid in water, Mobile Phase B: Acetonitrile with a gradient of T=0 min (95% A, 05% B); gradient to T=35.00 min (60% A, 40% B); T=35.01 min (02% A, 98% B) gradient to T=38.00 min (02% A, 98% B); T=38.01 min (95% A, 05% B); gradient to T=42 min (95% A, 05% B); Flow rate=80 ml/min; analysis time 42 min.

### Prep. HPLC Purification Method—Example 22

Purification was conducted with a Flash Chromatography-SELEKT system with a UV detector. Column 2*YMC C18,120 gm, 50 μm; compounds were eluted with, Mobile Phase A: 0.1% Formic acid in water, Mobile Phase B: Acetonitrile with a gradient of T=0 min (100% A, 00% B); gradient to T=05.00 min (100% A, 00% B); T=05.00 min (100% A, 00% B) gradient to T=35.00 min (80% A, 20% B); T=37.00 min (02% A, 98% B); gradient to T=37.01 min (100% A, 00% B); T=37.01 min (100% A, 00% B) gradient to T=40.00 min (100% A, 00% B); Flow rate=75 ml/min; analysis time 40 min.

### Prep. HPLC Purification Method—Example 23

Purification was conducted with a Shimadzu LC20AP purification system with a UV detector. Column: XTIMATE, C18,OBD (19×250)mm, 5 μm; compounds eluted with, Mobile Phase A: 0.1% Formic acid in water, Mobile Phase B: Acetonitrile with a gradient of T=0 min (85% A, 15% B); gradient to T=20.00 min (75% A, 25% B); T=20.01 min (02% A, 98% B) gradient to T=22.00 min (02% A, 98% B); T=22.01 min (85% A, 15% B); gradient to T=25 min (85% A, 15% B); Flow rate=20 ml/min; analysis time 25 min.

### Prep. HPLC Purification Method Example 24

Purification was conducted with a Shimadzu LC20AP purification system with a UV detector. Column: XTIMATE, C18, OBD (250×19)mm, 5 μm; compounds eluted with, Mobile Phase A: 0.1% Formic acid in water, Mobile Phase B: Acetonitrile with a gradient of T=0 min (100% A, 00% B); gradient to T=2 min (80% A, 20% B); T=02.00 min (80% A, 20% B); gradient of T=15.00 min (70% A, 30% B); T=15.01 min (02% A, 98% B) gradient of T=17.00 min (02% A, 98% B); T=17.01 min (100% A, 00% B); gradient to T=20 min (100% A, 00% B); Flow rate=20 ml/min; analysis time 20 min.

### Prep. HPLC Purification Method Example 25

Purification was conducted with a Flash Chromatography-SELEKT purification system with a UV detector. Column: YMC C18,120 gm, 50 μm; compounds eluted with, Mobile Phase A: 0.1% Formic acid in water, Mobile Phase B: Acetonitrile with a gradient of T=0 min (100% A, 00% B); gradient to T=25.00 min (90% A, 10% B); T=30.00 min (02% A, 98% B) gradient to T=35.00 min (02% A, 98% B); T=35.01 min (100% A, 00% B); gradient to T=40 min (100% A, 00% B); Flow rate=80 ml/min; analysis time 40 min.

### Prep. HPLC Purification Method—Example 26

Purification was conducted with a Shimadzu LC20AP purification system with a UV detector. Column: XTIMATE, C18, OBD 19×250 mm, 5 μm; compounds eluted with, Mobile Phase A: 0.1% Formic acid in water, Mobile Phase B: Acetonitrile with a gradient of T=0 min (93% A, 07% B); gradient to T=20.00 min (77% A, 23% B); T=20.01 min (02% A, 98% B) gradient to T=22.00 min (02% A, 98% B); T=22.01 min (93% A, 07% B); gradient to T=25 min (93% A, 07% B); Flow rate=20 ml/min; analysis time 20 min.

### Prep. HPLC Purification Method—Example 27

Purification was conducted with a Shimadzu Nexera purification system with a UV detector. Column: SUNFIRE C18 OBD 19*150 mm, 5 μm; compounds eluted with, Mobile Phase A: 0.1% Formic acid in water, Mobile Phase B: Acetonitrile: Tetrahydrofuran (90:10) with a gradient of T=0 min (100% A, 00% B); gradient to T=02.00 min (91% A, 09% B); T=02.00 min (91% A, 09% B) gradient to T=18.00 min (89% A, 11% B); T=18.01 min (02% A, 98% B); gradient to T=20 min (02% A, 98% B); T=20.01 min (100% A, 00% B) gradient to T=22.00 min (100% A, 00% B); Flow rate=21 ml/min.

### Prep. HPLC Purification Method—Example 28

Purification was conducted with a Flash Chromatography-SELEKT purification system with a UV detector. Column: YMC C18,120 gm, 50 μm; compounds eluted with, Mobile Phase A: 0.05% Formic acid in water, Mobile Phase B: Acetonitrile with a gradient of T=0 min (100% A, 00% B); gradient to T=05 min (100% A, 00% B); T=05.00 min (100% A, 00% B); gradient to T=30.00 min (75% A, 25% B); T=35.00 min (02% A, 98% B) gradient to T=40 min (100% A, 00% B); Flow rate=80 ml/min; analysis time 40 min.

### Prep. HPLC Purification Method—Example 29

Purification was conducted with a Waters 2545 purification system with a UV detector. Column: SUNFIRE C18 OBD (19×150)mm, 5 μm; compounds eluted with, Mobile Phase A: 0.05% Hydrochloric acid in water, Mobile Phase B: Acetonitrile: mobile phase-A(80:20) with a gradient of T=0 min (79% A, 21% B); gradient to T=17 min (79% A, 21% B); T=17.01 min (02% A, 98% B); gradient to T=19.00 min (02% A, 98% B); T=19.01 min (79% A, 21% B) gradient to T=22 min (79% A, 21% B); Flow rate=10 ml/min; analysis time 22 min.

### Prep. HPLC Purification Method—Example 30

Purification was conducted with a Shimadzu LC20AP purification system with a UV detector. Column: X-bridge Prep, C18,OBD (250×19)mm, 5 μm; compounds eluted with, Mobile Phase A: 0.05% Ammonium Hydroxide in water, Mobile Phase B: Acetonitrile with a gradient of T=0 min (75% A, 25% B); gradient to T=15 min (50% A, 50% B); T=15.01 min (02% A, 98% B); gradient to T=17.00 min (02% A, 98% B); T=17.01 min (75% A, 25% B) gradient to T=20 min (75% A, 25% B); Flow rate=19 ml/min; analysis time 20 min.

### Prep. HPLC Purification Method—Example 31

Purification was conducted with a Waters 2589 purification system with UV QDA detector. Column: XTIMATE, C18, OBD (250×19)mm, 5 μm; compounds eluted with, Mobile Phase A: 0.1% Formic acid in water, Mobile Phase B: Acetonitrile with a gradient of T=0 min (85% A, 15% B); gradient to T=15 min (70% A, 30% B); T=15.01 min (02% A, 98% B) gradient to T=17.00 min (02% A, 98% B); T=17.01 min (85% A, 15% B); gradient to T=20 min (85% A, 15% B); Flow rate=22 ml/min; analysis time 20 min.

### Prep. HPLC Purification Method—Example 32

Purification was conducted with a Waters 2545 purification system with a UV QDA detector. Column: XTIMATE, C18, OBD (250×19)mm, 5 μm and the compounds were eluted with, Mobile Phase A: 0.1% Formic acid in water, Mobile Phase B: Acetonitrile: Methanol (50:50) with a gradient of T=0 min (62% A, 38% B); gradient to T=15 min (61% A, 39% B); T=15.01 min (02% A, 98% B) gradient to T=17.00 min (02% A, 98% B); T=17.01 min (62% A, 38% B); gradient to T=22 min (62% A, 38% B); Flow rate=20 ml/min; analysis time 22 min.

### Prep. HPLC Purification Method—Example 33

Purification was conducted with a Waters 2545 purification system with a UV QDA detector. Column: Xtimate C18 (250 mm×21.2 mm×5 μm; compound eluted with, Mobile Phase A: 0.05% formic acid in water, Mobile Phase B: Acetonitrile with a gradient of T=0 min (86% A, 14% B); gradient to T=15.00 min (75% A, 25% B); T=15.01 min (2% A, 98% B); gradient to T=17.00 min (2% A, 98% B); T=17.01 min (86% A, 14% B) to T=25.00 min (86% A, 14% B);); Flow rate=20 ml/min; analysis time 25 min.

### Prep. HPLC Purification Method—Example 34

Purification was conducted with a Waters 2545 purification system with a UV QDA detector. Column: SHIMPACK GIST C18, (250×19)mm, 5 μm; compounds eluted with, Mobile Phase A: 0.1% Formic acid in water, Mobile Phase B: Acetonitrile with a gradient of T=0 min (65% A, 35% B); gradient to T=20.00 min (65% A, 35% B); Flow rate=15 ml/min; T=20.01 min (02% A, 98% B) gradient to T=22.00 min (02% A, 98% B); Flow rate=19 ml/min; T=22.01 min (65% A, 35% B); gradient to T=25 min (65% A, 35%).

### Prep. HPLC Purification Method—Example 35

Purification was conducted with a Waters 2545 purification system with a UV detector. Column: SHIMPACK, C18, OBD (250×19)mm, 5 μm; compounds eluted with, Mobile Phase A: 0.1% Formic acid in water, Mobile Phase B: Acetonitrile:Methanol:Isopropylalcohol (65:25:10) with a gradient of T=0 min (65% A, 35% B); gradient to T=24.00 min (65% A, 35% B); T=24.01 min (02% A, 98% B) gradient to T=26.00 min (02% A, 98% B); T=26.01 min (65% A, 35% B); gradient to T=28 min (65% A, 35% B); Flow rate=20 ml/min; analysis time 28 min.

### Prep. HPLC Purification Method—Example 36

Purification was conducted with a Shimadzu LC20AP purification system with a UV detector. Column XBRIDGE, C18, OBD (19×250)mm, 5 μm; compounds eluted with, Mobile Phase A: 0.05% Hydrochloric acid in water, Mobile Phase B: Acetonitrile with a gradient of T=0 min (78% A, 22% B); gradient to T=17 min (65% A, 35% B); T=17.01 min (02% A, 98% B); gradient to T=19.00 min (02% A, 98% B); T=19.01 min (78% A, 22% B) to T=22 min (78% A, 22% B); Flow rate=20 ml/min; analysis time 22 min.

### Preparation of Intermediates

### (4-Aminoisoindolin-2-yl)(2-(benzyloxy)-4,6-dihydroxyphenyl)methanone: Intermediate 1

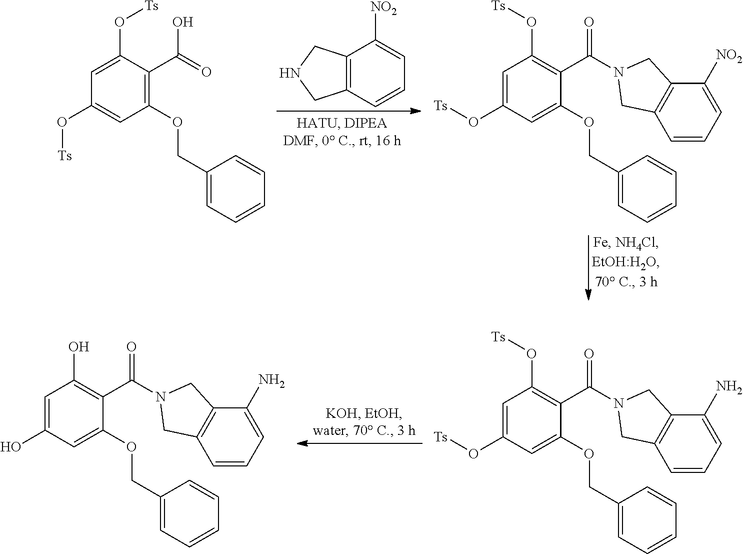

### 5-(Benzyloxy)-4-methyl-6-(4-nitroisoindoline-2-carbonyl)-1,3-phenylene bis(4-methylbenzenesulfonate)

A stirred solution 2-(benzyloxy)-4,6-bis(tosyloxy)benzoic acid (Intermediate 4) (2.0 g, 3.56 mmol, 1 eq.) in DMF (10 mL) at 0° C. was treated with HATU (1.73 g, 4.55 mmol, 1.3 eq.) and DIPEA (0.6 ml, 4.63 mmol, 1.5 eq.). The reaction mixture was stirred at 0° C. for 10 min. 4-Nitroisoindoline (CAS:748735-45-7) (0.635 g, 3.87 mmol, 1.1 eq.) was added at 0° C. and the reaction mixture stirred at room temperature for 16 h. The reaction mixture was poured into ice cold water (200 mL) and extracted using ethyl acetate (3×220 mL), dried over Na₂SO₄, filtered, and concentrated under vacuum. The crude material was purified by flash column chromatography (product eluted with 23.6% ethyl acetate in hexane) to give the title compound as a yellow solid (1.2 g, Yield: 47.7%).

LCMS (Method A): 2.565 min, MS: ES+715.23 (M+1).

### 4-(4-Aminoisoindoline-2-carbonyl)-5-(benzyloxy)-1,3-phenylene bis(4-methylbenzenesulfonate)

A stirred solution of 5-(benzyloxy)-4-methyl-6-(4-nitroisoindoline-2-carbonyl)-1,3-phenylene bis(4-methylbenzenesulfonate) (1.2 g, 1.68 mmol, 1 eq.) in EtOH: H₂O (2:1) at room temperature was treated with Fe (0.56 g, 10.03 mmol, 6 eq.) and NH₄Cl (1.35 g, 25.23 mmol, 15 eq.). The reaction mixture was heated at 70° C. and stirred for 3 h. The reaction mixture was cooled and passed through a celite bead using ethyl acetate (20 mL) and poured into ice cold water (20 mL) and extracted in ethyl acetate (2×20 mL). The combined organic layer was dried over Na₂SO₄, filtered, and concentrated under vacuum yielding the title compound as a brown sticky solid (0.75 g, Yield: 65.2%).

LCMS (Method A): 2.431 min, 2.487 min, MS: ES+685.2 (M+1).

### (4-Aminoisoindolin-2-yl)(2-(benzyloxy)-4,6-dihydroxyphenyl)methanone: Intermediate 1

A stirred solution of 4-(4-aminoisoindoline-2-carbonyl)-5-(benzyloxy)-1,3-phenylene bis(4-methylbenzenesulfonate) (0.7 g, 1.02 mmol, 1 eq.) in EtOH:H₂O (4:1) at room temperature was treated with KOH (2.29 g, 40.89 mmol, 40 eq.). The reaction mixture was heated at 60° C. and stirred for 2 h. The reaction mixture was acidified using saturated KHSO₄ solution and extracted into ethyl acetate (3×45 mL). The combined organic layer was dried over Na₂SO₄, filtered, and concentrated under vacuum. The crude material was purified by normal phase column chromatography (product eluted with 5% MeOH in DCM) yielding the title compound as an orange solid (0.098 g, Yield: 25.5%).

¹H NMR (DMSO-d6, 400 MHz), compound is a mixture of rotamers: δ 4.33-4.39 (m, 2H), 4.52-4.63 (m, 2H), 5.03-5.08 (m, 3H), 5.17 (s, 1H), 5.99-6.01 (2 singlets, 2H), 6.37-6.52 (m, 2H), 6.91-6.98 (m, 1H), 7.22-7.30 (m, 5H), 9.45-9.54 (m, 2H). LCMS (Method A): 1.491 min, MS ES+377.22 (M+1).

### (4-Aminoisoindolin-2-yl)(2-(benzyloxy)-4,6-dihydroxy-3-methylphenyl)methanone: Intermediate 2

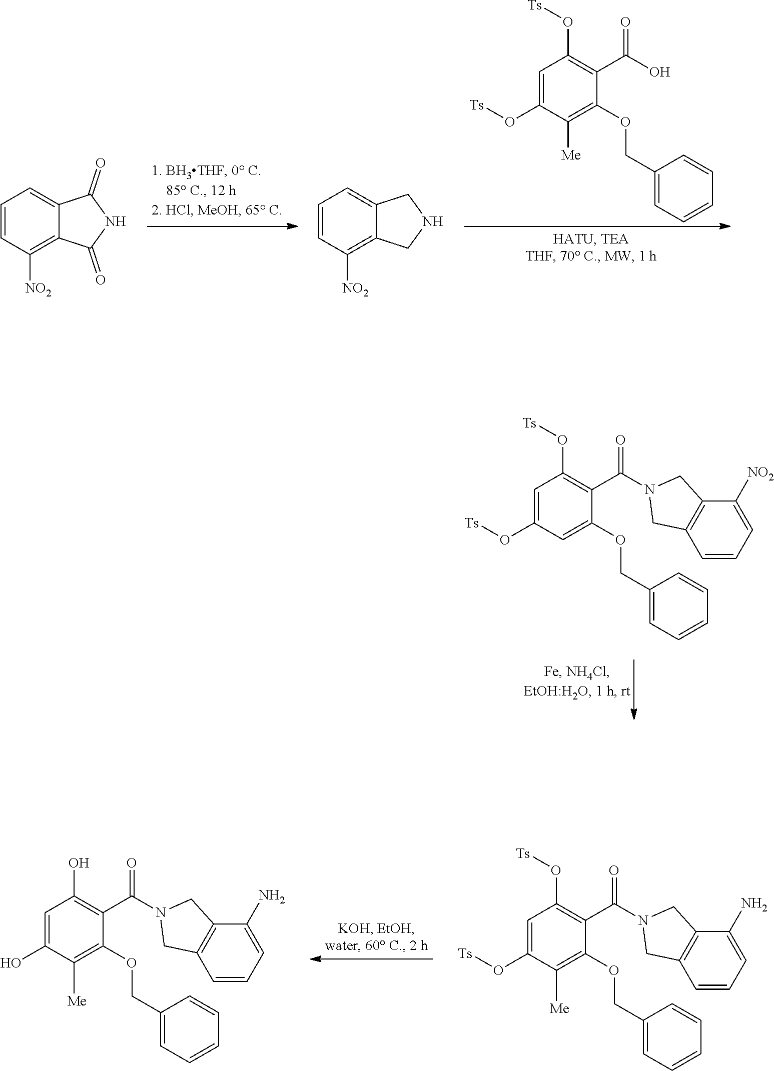 [SMILES: O=C1NC(=O)c2c1cccc2[N+](=O)[O-] ; O=[N+]([O-])c1cccc2c1CNC2 ; Cc1ccc(S(=O)(=O)Oc2cc(OS(=O)(=O)c3ccc(C)cc3)c(C(=O)O)c(OCc3ccccc3)c2C)cc1 ; Cc1ccc(S(=O)(=O)Oc2cc(OCc3ccccc3)c(C(=O)N3Cc4cccc([N+](=O)[O-])c4C3)c(OS(=O)(=O)c3ccc(C)cc3)c2)cc1 ; Cc1c(O)cc(O)c(C(=O)N2Cc3cccc(N)c3C2)c1OCc1ccccc1 ; Cc1ccc(S(=O)(=O)Oc2cc(OS(=O)(=O)c3ccc(C)cc3)c(C(=O)N3Cc4cccc(N)c4C3)c(OCc3ccccc3)c2C)cc1]

### 4-Nitro-isoindoline

Performed in 2 parallel batches, each of 6.5 g scale. A stirred solution of 4-nitro-isoindoline-1,3-dione (CAS: 603-62-3) (6.5 g, 33.8 mmol, 1.0 eq.) in THF (130 mL) at 0° C. was treated dropwise with 1 M Borane.THF (136.5 mL, 4 eq.). The reaction mixture was heated to 80° C. and stirred (16 h). The resulting reaction was allowed to cool to room temperature, MeOH (13 mL) and 6N HCl (32.5 mL) were added dropwise. The resulting reaction mixture was further heated to 65° C. and stirred for 1 h. The reaction mixture was concentrated under vacuum and the crude material neutralized using sat. NaHCO₃ solution. As the desired product was water soluble, the aqueous layer was lyophilized. Crude material was purified by reverse phase column chromatography (product eluted with 63% MeCN in water) yielding the title compound as a brown solid (1.0 g, Yield: 7.9%).

¹H NMR (DMSO-d6, 400 MHz): δ 4.63 (s, 2H), 4.93 (s, 2H), 7.70 (t, J=8 Hz, 16 Hz, 1H), 7.87 (d, J=7.6 Hz, 1H), 8.22 (d, J=8 Hz, 1H), 10.24 (bs, 2H). LCMS (Method A): 0.389 min, MS: ES+165.2 (M+1).

### 5-(Benzyloxy)-4-methyl-6-(4-nitroisoindoline-2-carbonyl)-1,3-phenylene bis(4-methylbenzenesulfonate)

Performed in 3 parallel batches, each of 0.5 g scale. 2-(benzyloxy)-3-methyl-4,6-bis(tosyloxy)benzoic acid (Intermediate 6) (0.5 g, 0.86 mmol, 1 eq.) in DMF (5 mL), HATU (0.653 g, 1.72 mmol, 2 eq.) and TEA (0.095 g, 0.94 mmol, 1.1 eq.) were added to a microwave vial at 0° C. 4-Nitroisoindoline (0.169 g, 1.03 mmol, 1.2 eq.) was added and the reaction mixture heated at 70° C. under microwave irradiation for 1 hr. The reaction mixture was poured into ice cold water (100 mL) and extracted with ethyl acetate (3×300 mL). The combined organic layer was dried over Na₂SO₄, filtered, and concentrated under vacuum. The crude material was purified by flash column chromatography (product eluted in 32% ethyl acetate in hexane yielding the title compound as a pale yellow solid (1.0 g, Yield: 34.6%).

LCMS (Method A): 2.802 min, MS: ES+729.1 (M+1) 751.0 (M+23).

### (4-(4-Aminoisoindoline-2-carbonyl)-5-(benzyloxy)-6-methyl-1,3-phenylene bis(4-methylbenzenesulfonate))methanone

A stirred solution of 5-(benzyloxy)-4-methyl-6-(4-nitroisoindoline-2-carbonyl)-1,3-phenylene bis(4-methylbenzenesulfonate) (0.5 g, 0.68 mmol, 1 eq.) in EtOH: H₂O (6 mL; 5:1 ratio) at room temperature was treated with Fe powder (0.230 g, 4.12 mmol, 6 eq.) and NH₄Cl (0.551 g, 10.3 mmol, 15 eq.). The reaction mixture was heated to 80° C. and stirred for 2 h. The reaction mixture was allowed to cool to room temperature filtered through a celite bed, washed with ethyl acetate (200 mL) and the combined filtrate poured into ice cold water (100 mL) and extracted in ethyl acetate (3×300 mL). The combined organic layer was dried over Na₂SO₄, filtered, and concentrated under vacuum. The crude material was triturated using n-pentane (3×30 mL) and diethyl ether (3×30 mL) followed by drying yielding the title compound as a pale yellow solid (0.9 g, Yield: 82.8%).

LCMS (Method A): 2.665 min, 36.18%, 254 nm MS: ES+699.2 (M+1); 2.768 min, 57.34%, 254 nm MS: ES+699.2 (M+1).

### (4-Aminoisoindolin-2-yl)(2-(benzyloxy)-4,6-dihydroxy-3-methylphenyl)methanone: Intermediate 2

A stirred solution of (4-(4-aminoisoindoline-2-carbonyl)-5-(benzyloxy)-6-methyl-1,3-phenylene bis(4-methylbenzenesulfonate))methanone (0.5 g, 0.71 mmol, 1 eq.) in EtOH: H₂O (2:1) at room temperature was treated with KOH (1.14 g, 20.3 mmol, 40 eq.). The reaction mixture was heated to 60° C. and stirred for 2 h. The reaction mixture was allowed to cool to room temperature, acidified using saturated solution of KHSO₄ (pH −6-7) and extracted into ethyl acetate (3×100 mL). The combined organic layer was dried over Na₂SO₄, filtered, and concentrated under vacuum. The crude material was purified by column chromatography (product eluted with 2.1% MeOH in DCM) yielding the title compound as an off white solid (0.2 g, Yield: 71.6%).

¹H NMR (DMSO-d6, 400 MHz): δ 1.97-1.98 (d, J=2.0 Hz, 3H), 4.31-4.40 (m, 2H), 4.47-4.56 (m, 1H), 4.66-4.67 (m, 1H), 4.72-4.74 (m, 1H), 4.90-4.93 (m, 1H), 5.05 (s, 1H), 5.20 (s, 1H), 6.30 (d, J=2.4 Hz, 1H), 6.36-6.53 (m, 2H), 6.91-6.98 (m, 1H), 7.28-7.38 (m, 5H), 9.47-9.54 (m, 2H). LCMS (Method A): 1.681 min, MS: ES+391.3.

### 4-Aminoisoindolin-2-yl)(2-(cyclohexylmethoxy)-4,6-dihydroxy-3-methylphenyl)methanone: Intermediate 3

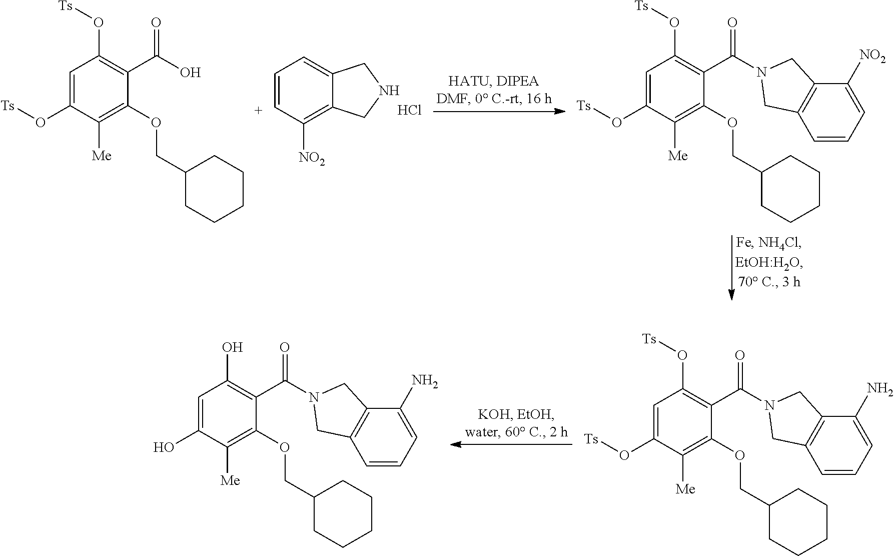 [SMILES: Cc1ccc(S(=O)(=O)Oc2cc(OS(=O)(=O)c3ccc(C)cc3)c(C(=O)O)c(OCC3CCCCC3)c2C)cc1 ; O=[N+]([O-])C1=C2CNCC2=CC=[C+]1 ; Cl ; Cc1ccc(S(=O)(=O)Oc2cc(OS(=O)(=O)c3ccc(C)cc3)c(C(=O)N3Cc4cccc([N+](=O)[O-])c4C3)c(OCC3CCCCC3)c2C)cc1 ; Cc1c(O)cc(O)c(C(=O)N2Cc3cccc(N)c3C2)c1OCC1CCCCC1 ; Cc1ccc(S(=O)(=O)Oc2cc(OS(=O)(=O)c3ccc(C)cc3)c(C(=O)N3Cc4cccc(N)c4C3)c(OCC3CCCCC3)c2C)cc1]

### 5-(Cyclohexylmethoxy)-4-methyl-6-(4-nitroisoindoline-2-carbonyl)-1,3-phenylene bis(4-methylbenzenesulfonate)

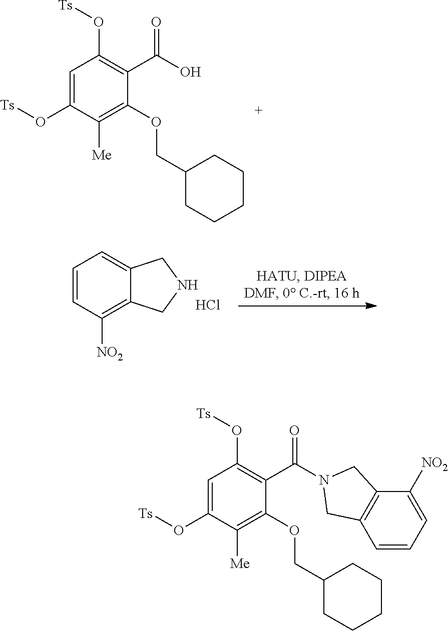 [SMILES: Cc1ccc(S(=O)(=O)Oc2cc(OS(=O)(=O)c3ccc(C)cc3)c(C(=O)O)c(OCC3CCCCC3)c2C)cc1 ; O=[N+]([O-])c1cccc2c1CNC2 ; Cl ; Cc1ccc(S(=O)(=O)Oc2cc(OS(=O)(=O)c3ccc(C)cc3)c(C(=O)N3Cc4cccc([N+](=O)[O-])c4C3)c(OCC3CCCCC3)c2C)cc1]

A stirred solution 2-(cyclohexylmethoxy)-3-methyl-4,6-bis(tosyloxy)benzoic acid (Intermediate 5) (2 g, 3.40 mmol, 1 eq.) in DMF (20 mL) at 0° C. was treated with HATU (1.93 g, 5.07 mmol, 1.5 eq.) and DIPEA (0.87 g, 6.74 mmol, 2 eq.) and stirred for 15 min. 4-nitroisoindoline hydrochloride (0.88 g, 4.4 mmol, 1.3 eq.) was added and the resulting reaction mixture stirred at room temperature for 16 h. The reaction mixture was poured into ice cold water (80 mL) and extracted using ethyl acetate (3×60 mL). The combined organic layer was washed with ice cold water (3×120 mL), dried over Na₂SO₄, filtered, and concentrated under vacuum. The crude material was purified by flash chromatography (product eluted in 5% MeOH in DCM yielding the title compound) as a yellow solid (2.0 g, Yield: 80.1%).

LCMS (Method D): 3.165 min, 3.182 min, MS: ES+735.3 (M+1).

### 4-(4-Aminoisoindoline-2-carbonyl)-5-(cyclohexylmethoxy)-6-methyl-1,3-phenylene bis(4-methylbenzenesulfonate)

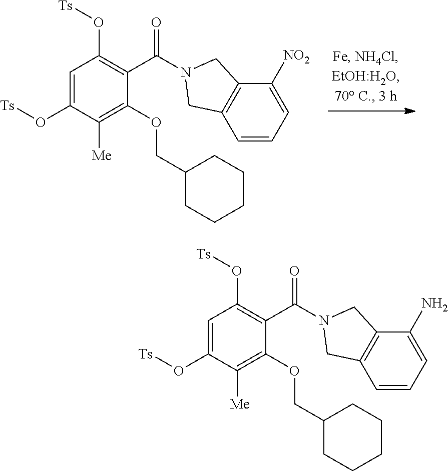

Conducted in two parallel batches of 1.1 g scale: A stirred solution of 5-(cyclohexylmethoxy)-4-methyl-6-(4-nitroisoindoline-2-carbonyl)-1,3-phenylene bis(4-methylbenzenesulfonate) (1.1 g, 1.5 mmol, 1 eq.) in EtOH: H₂O (2:1) (10 mL) at room temperature was treated with Fe powder (0.5 g, 8.96 mmol, 6 eq.) and NH₄Cl (1.2 g, 22.3 mmol, 15 eq.). The reaction mixture was heated to 70° C. and stirred for 3 h, cooled, and filtered through a celite bed which was washed with ethyl acetate (200 mL). The organic layer was poured into ice cold water (80 mL) and further extracted with ethyl acetate (3×50 mL). The combined organic layers were dried over Na₂SO₄, filtered, and concentrated under vacuum yielding the title compound as a yellow solid (1.6 g, Yield: 75.8%). The crude material was used in the next step without purification.

LCMS (Method D): 3.154 min, 3.035 min, MS: ES+705.3 (M+1).

### 4-Aminoisoindolin-2-yl)(2-(cyclohexylmethoxy)-4,6-dihydroxy-3-methylphenyl)methanone: Intermediate 3

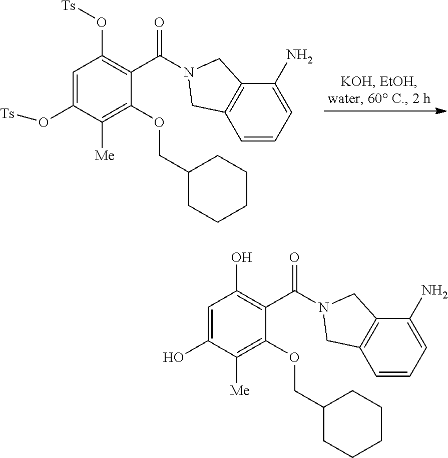

Conducted in 2 parallel batches of 0.4 g scale: A stirred solution of 4-(4-aminoisoindoline-2-carbonyl)-5-(cyclohexylmethoxy)-6-methyl-1,3-phenylene bis(4-methylbenzenesulfonate) (0.4 g, 0.56 mmol, 1 eq.) in EtOH: H₂O (2:1) (6 mL) at room temperature was treated with KOH (1.27 g, 22.7 mmol, 40 eq.). The reaction mixture was heated to 60° C. and stirred for 2 h, cooled, poured into ice cold water (100 mL) and acidified using saturated KHSO₄ solution. The product was extracted into ethyl acetate (3×60 mL), the combined organic layer dried over Na₂SO₄, filtered, and concentrated under vacuum. The crude material was purified by flash chromatography (product eluted with 7% MeOH in DCM) yielding the title compound as an off white solid (0.2 g, Yield: 44.4%).

High temperature ¹H NMR (DMSO-d6, 400 MHz, 349.5K): δ 1.0-1.08 (m, 2H), 1.11-1.29 (m, 3H), 1.57-1.69 (m, 6H), 1.95 (s, 3H), 3.41-3.66 (m, 2H), 4.30-4.44 (m, 2H), 4.56-4.67 (m, 2H), 4.86-4.96 (m, 2H), 6.23 (s, 1H), 6.40-6.55 (m, 2H), 6.92-6.97 (m, 1H), 9.02 (s, br, 1H), 9.11 (s, br, 1H). LCMS (Method A): 1.917 min, 98.84%, 254 nm, MS: ES+397.5 (M+1).

Analytical HPLC Method C: 6.457 min, 97.8%, 254 nm.

### 2-(Benzyloxy)-4,6-bis(tosyloxy)benzoic acid: Intermediate 4

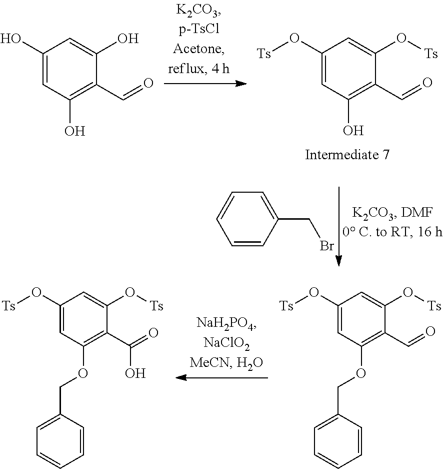 [SMILES: O=Cc1c(O)cc(O)cc1O ; Cc1ccc(S(=O)(=O)Oc2cc(O)c(C=O)c(OS(=O)(=O)c3ccc(C)cc3)c2)cc1 ; BrCc1ccccc1 ; Cc1ccc(S(=O)(=O)Oc2cc(OCc3ccccc3)c(C(=O)O)c(OS(=O)(=O)c3ccc(C)cc3)c2)cc1 ; Cc1ccc(S(=O)(=O)Oc2cc(OCc3ccccc3)c(C=O)c(OS(=O)(=O)c3ccc(C)cc3)c2)cc1]

### 5-(Benzyloxy)-4-formyl-1,3-phenylene bis(4-methylbenzenesulfonate)

A solution of 2,4,6-trihydroxybenzaldehyde (CAS 487-70-7) (5 g, 32.2 mmol, 1.0 eq.) in acetone (200 mL) at rt was treated with K₂CO₃ (22.27 g, 161 mmol, 5.0 eq.) and p-toluene sulphonyl chloride (12.90, 67.7 mmol, 2.1 eq.). The reaction mixture was heated at 60° C. for 4 h then allowed to cool to room temperature and concentrated under reduced pressure. The crude material was diluted with water (300 mL) and extracted in EtOAc (3×400 mL). The combined organic layer was washed with brine solution (200 mL), dried over sodium sulphate, and concentrated under reduced pressure to give crude material which was purified by column chromatography using silica gel (eluting product using 8.7% ethyl acetate in hexane) yielding 4-formyl-5-hydroxy-1,3-phenylene bis(4-methylbenzenesulfonate) (Intermediate 7) as a yellow solid (5.9 g, Yield: 39.3%).

¹H NMR (DMSO-d⁶, 400 MHz): δ 2.44 (d, J=2.4 Hz, 6H), 6.38 (d, J=2.4 Hz, 1H), 6.66 (d, J=2.4 Hz, 1H), 7.47-7.54 (m, 4H), 7.69 (d, J=8.0 Hz, 2H), 7.78 (d, J=8.4 Hz, 2H), 9.89 (s, 1H), 11.52 (s, 1H). LCMS (Method A): 2.441 min, MS: ES+461.30 (M−1)

A solution of 4-formyl-5-hydroxy-1,3-phenylene bis(4-methylbenzenesulfonate) (Intermediate 7) (20 g 1.0 eq.) in DMF (200 mL) at 0° C. temperature was treated with K₂CO₃ (3.0 eq.) and benzylbromide (6.72 g, 1.3 eq.). The reaction mixture was stirred at room temperature for 16 h. The resulting reaction mixture was diluted with ethyl acetate (400 mL) and washed with chilled brine solution (3×500 mL). The combined organic layer was dried over Na₂SO₄, filtered, and concentrated under reduced pressure. The crude material (21.5 g) was purified by column chromatography using silica gel (eluting product with 16% ethyl acetate in hexane) to give the title compound 5-(benzyloxy)-4-formyl-1,3-phenylene bis(4-methylbenzenesulfonate) as an off-white solid (20 g, Yield: 83%).

¹H NMR (DMSO-d6, 400 MHz): δ ppm 2.44 (d, J=2.4 Hz, 6H), 5.15 (s, 2H), 6.46 (d, J=2 Hz, 1H), 7.07 (d, J=2 Hz, 1H), 7.39-7.41 (m, 5H), 7.46-7.51 (m, 4H), 7.65 (d, J=8.4 Hz, 2H), 7.74 (d, J=8.4 Hz, 2H), 10.00 (s, 1H). LCMS (Method A): 2.651 min, MS: ES+574.9 (M+23).

### 2-(Benzyloxy)-4,6-bis(tosyloxy)benzoic acid: Intermediate 4

A solution of 5-(benzyloxy)-4-formyl-1,3-phenylene bis(4-methylbenzenesulfonate) (20 g, 1.0 eq.) in MeCN: Water (200 mL) at room temperature was treated with NaClO₂ (12.73 g, 3.7 eq.) and NaH₂PO₄ (9.12 g, 2.0 eq.). The resulting reaction mixture was stirred at room temperature for 16 h. MeCN was removed under vacuum and the resulting aqueous solution diluted with water (300 mL) and neutralized with dil. HCl (pH −7) then extracted with ethyl acetate (2×500 mL). The combined organic layer was dried over Na₂SO₄, filtered, and concentrated under reduced pressure. Crude material was triturated using diethyl ether (2×200 mL) and n-pentane (100 mL) to give the title compound as an off-white solid (20.0 g, Yield: 97%).

¹H NMR (DMSO-d6, 400 MHz): δ ppm 2.43 (s, 6H), 5.03 (s, 2H), 6.52 (d, J=2 Hz, 1H), 6.85 (s, 1H), 7.32-7.37 (m, 5H), 7.47-7.49 (q, J=6.4, 8 Hz, 4H), 7.70 (t, J=8.4 Hz, 4H), 13.48 (bs, 1H). LCMS (Method A): 2.344 min, MS: ES+568.91 (M+1).

### 2-(Cyclohexylmethoxy)-3-methyl-4,6-bis(tosyloxy)benzoic acid: Intermediate 5

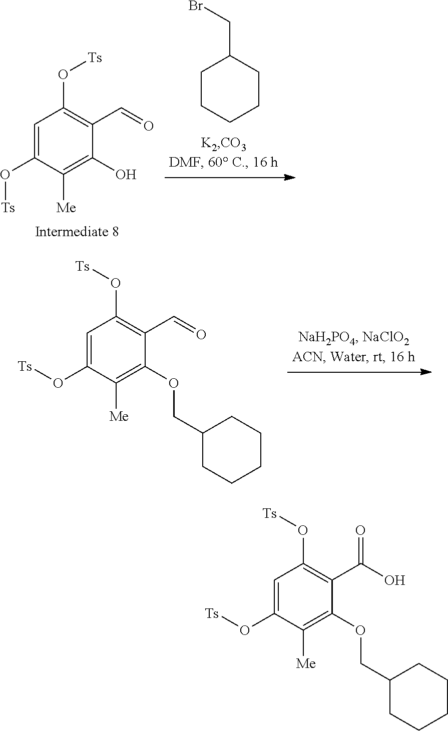 [SMILES: Cc1ccc(S(=O)(=O)Oc2cc(OS(=O)(=O)c3ccc(C)cc3)c(C=O)c(O)c2C)cc1 ; BrCC1CCCCC1 ; Cc1ccc(S(=O)(=O)Oc2cc(OS(=O)(=O)c3ccc(C)cc3)c(C=O)c(OCC3CCCCC3)c2C)cc1 ; Cc1ccc(S(=O)(=O)Oc2cc(OS(=O)(=O)c3ccc(C)cc3)c(C(=O)O)c(OCC3CCCCC3)c2C)cc1]

### 5-(Cyclohexylmethoxy)-4-formyl-6-methyl-1,3-phenylene bis(4-methylbenzenesulfonate)

A stirred solution of 4-formyl-5-hydroxy-6-methyl-1,3-phenylene-bis(4-methylbenzenesulfonate) (Intermediate 8) (2.0 g, 4.19 mmol, 1.0 eq.) in DMF (20 mL) at 0° C. was treated with K₂CO₃ (2.89 g, 20.9 mmol, 5.0 eq.) and bromomethyl cyclohexane (1.11 g, 6.29 mmol, 1.5 eq.) and stirred for 10 mins. The reaction mixture was heated to 60° C. and stirred for 16 h. The reaction mixture was poured into ice-cold water (20 mL) and extracted in ethyl acetate (3×30 mL). The combined organic layer was dried over Na₂SO₄, filtered, and concentrated under vacuum. Crude material was purified by flash chromatography (product eluted with 12% ethyl acetate in hexane) yielding the title compound (1.4 g, Yield: 58.3%).

¹H NMR (DMSO-d6, 400 MHz): Compound is a mixture of rotamers: δ 0.97-1.00 (m, 2H), 1.11-1.26 (m, 3H), 1.67-1.73 (m, 5H), 1.77 and 1.83 (singlets, 3H), 2.33-2.45 (singlets, 6H), 3.49 and 3.70 (m, 2H), 5.77 (s, 1H), 6.44 and 6.70 (singlets, 1H), 7.48-7.53 (m, 4H), 7.71-7.81 (m, 4H), 9.86 and 9.90 (singlets, 1H). LCMS (Method D): 3.250 min, MS: ES+573.13 (M+1).

### 2-(Cyclohexylmethoxy)-3-methyl-4,6-bis(tosyloxy)benzoic acid: Intermediate 5

A solution of 5-(cyclohexylmethoxy)-4-formyl-6-methyl-1,3-phenylene bis(4-methylbenzenesulfonate) (1.4 g, 2.44 mmol, 1.0 eq.) in MeCN:Water (1:1) (15 mL) at room temperature was treated with NaH₂PO₄ (1.02 g, 8.55 mmol, 3.5 eq.) and NaClO₂ (1.10 g, 12.22 mmol, 5.0 eq.) and stirred for 16 h. The mixture was evaporated, and the crude material diluted with water (30 mL) and extracted with ethyl acetate (3×30 mL). The combined organic layer was dried over Na₂SO₄, filtered, and concentrated under reduced pressure. The crude material was purified by flash chromatography (eluting product using 15% ethyl acetate in hexane) yielding the title compound (0.950 g, Yield: 66.4%).

¹H NMR (DMSO-d6, 400 MHz): δ0.95-1.10 (m, 2H), 1.11-1.22 (m, 3H), 1.61-1.69 (m, 6H), 1.82 (s, br, 3H), 2.44 (s, br, 6H), 3.54 (m, br, 2H), 4.03 (q, J=6.8 Hz, 14.0 Hz, 2H), 6.69 (s, 1H), 7.49-7.52 (m, 4H), 7.70-7.73 (m, 4H), 13.59 (s, 1H). LCMS (Method E): 2.657 min, MS: ES+589.2 (M+1)

### 2-(Benzyloxy)-3-methyl-4,6-bis(tosyloxy)benzoic acid: Intermediate 6

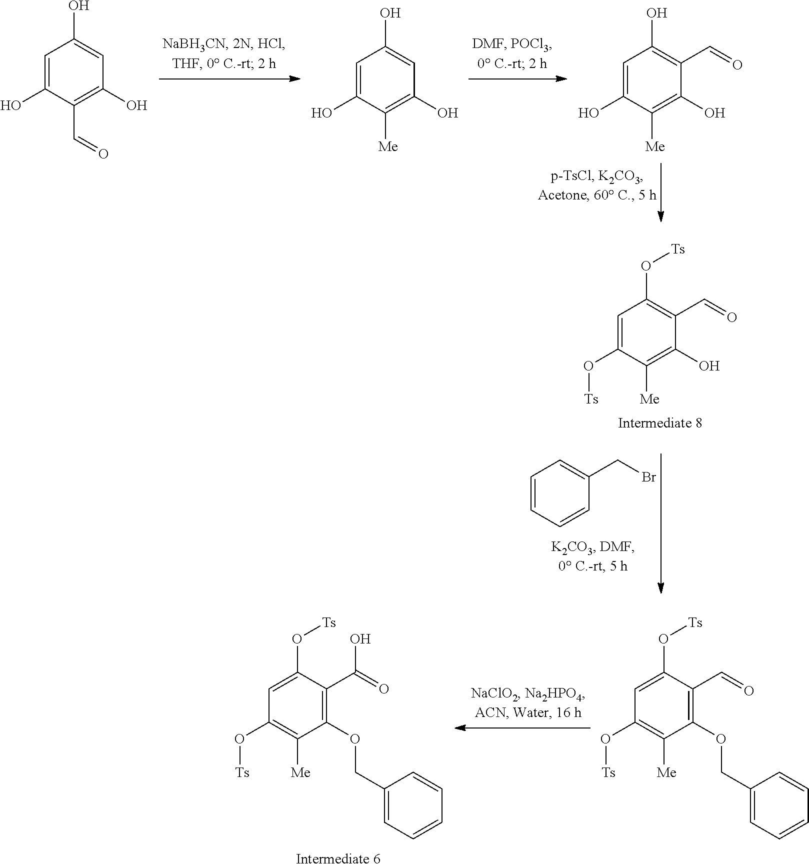

### 2-Methylbenzene-1,3,5-triol

Carried out in 6 batches in parallel. A stirred solution of 2,4,6-trihydroxybenzaldehyde (CAS: 487-70-7; 40 g, 260 mmol, 1 eq.) in THF (800 mL) at room temperature was treated with NaBH₃CN (81.61 g, 1298 mmol, 5 eq.). The resulting reaction mixture was allowed to cool to 0° C. and 2N HCl solution (400 mL) was added dropwise to the reaction mixture at 0° C. The resulting reaction mixture allowed to stir at room temperature for 3 h, then concentrated under vacuum; the crude material was poured into water (5 L) and extracted using ethyl acetate (3×4 L). The combined organic layer was dried over Na₂SO₄ and concentrated under vacuum. The crude material (216 g) was purified by column chromatography (product eluted in 5% MeOH in DCM) yielding the title compound as an off white solid (112 g, Yield: 51%).

¹H NMR (DMSO-d6, 400 MHz): δ ppm 1.81 (s, 3H), 5.77 (s, 2H), 8.69 (s, 1H), 8.81 (s, 2H). LCMS (Method A): 0.636 min, MS: ES+140.8 (M+1).

### 2,4,6-Tri hydroxy-3-methylbenzaldehyde

Carried out in 2 batches in parallel. A stirred solution of 2-methylbenzene-1,3,5-triol (25 g, 178.6 mmol, 1 eq.) in DMF (250 mL) was cooled to 0° C. POCl₃(30.11 g, 196.37 mmol, 1.1 eq.) was added dropwise at 0° C. and the resulting reaction mixture stirred at room temperature for 2 h. The reaction mixture was poured into ice cold water (2 L) and extracted using ethyl acetate (4×2.2 L). The combined organic layer was dried over Na₂SO₄ and concentrated under vacuum; the crude material was purified by column chromatography (product eluted in 3% MeOH in DCM) yielding the title compound as an off white solid (40 g, Yield: 70%).

¹H NMR (DMSO-d6, 400 MHz): δ ppm 1.88 (s, 3H), 6.00 (s, 1H), 9.94 (s, 1H), 10.55 (s, 1H), 10.63 (s, 1H), 12.33 (s, 1H). LCMS (Method A): 1.285 min, MS: ES+168.8 (M+1).

### 4-Formyl-5-hydroxy-6-methyl-1,3-phenylene bis(4-methylbenzenesulfonate) (Intermediate 8_1

Carried out in 4 parallel batches. A stirred solution of 2,4,6-trihydroxy-3-methylbenzaldehyde (10 g, 59.88 mmol, 1 eq.) in acetone (200 mL) at room temperature was treated with K₂CO₃ (24.79 g, 179.37 mmol, 3 eq.). The resulting reaction mixture was allowed to stir at room temperature for 15-20 min. p-Toluene sulphonyl chloride (17.10 g, 89.78 mmol, 1.5 eq.) was then added at room temperature. The reaction mixture was heated to 60° C. and stirred for 5 h, then concentrated under reduced pressure, poured into water (2 L) and extracted using ethyl acetate (3×2.2 L). The combined organic layer was dried over Na₂SO₄ and concentrated under vacuum. The crude material was purified by column chromatography (product eluted in 10% EtOAc in hexane) yielding the title compound as a pale-yellow solid (16 g, Yield: 14%).

¹H NMR (DMSO-d6, 400 MHz): δ ppm 1.76 (s, 3H), 2.43 (s, 3H), 2.45 (s, 3H), 6.44 (s, 1H), 7.50-7.52 (m, 4H), 7.75 (d, J=8 Hz, 2H), 7.79 (d, J=8 Hz, 2H), 9.85 (s, 1H), 11.8 (bs, 1H). LCMS (Method A): 2.591 min, MS: ES+476.5 (M+1).

### 5-(Benzyloxy)-4-formyl-6-methyl-1,3-phenylene bis(4-methylbenzenesulfonate)

A stirred solution of 4-formyl-5-hydroxy-6-methyl-1,3-phenylene bis(4-methylbenzenesulfonate) (16 g, 33.61 mmol, 1 eq.) in DMF (160 mL) at 0° C. was treated with K₂CO₃ (13.93 g, 100.79 mmol, 3 eq.) followed by benzyl bromide (6.33 g, 37.03 mmol, 1.10 eq.). The resulting reaction mixture was stirred at room temperature for 16 h, then poured into water (800 mL) and extracted using ethyl acetate (3×500 mL). The combined organic layer was washed with brine (2×800 mL), dried over Na₂SO₄, filtered, and concentrated under vacuum. The crude material was purified by column chromatography (product eluted in 16% EtOAc in Hexane) yielding the title compound as an off-white solid (16 g, Yield: 84%).

¹H NMR (DMSO-d6, 400 MHz): δ ppm 1.82 (s, 3H), 2.44 (s, 3H), 2.46 (s, 3H), 4.81 (s, 2H), 6.76 (s, 1H), 7.31-7.38 (m, 5H), 7.52 (t, J=8.8 Hz, 4H), 7.71-7.77 (m, 4H), 9.87 (m, 1H). LCMS (Method A): 2.699 min, MS: ES+588.9 (M+23).

### 2-(Benzyloxy)-3-methyl-4,6-bis(tosyloxy)benzoic acid: Intermediate 6

A stirred solution of 5-(benzyloxy)-4-formyl-6-methyl-1,3-phenylene bis(4-methylbenzenesulfonate) (16 g, 28.26 mmol, 1 eq.) in MeCN: Water (1:1) (160 mL) at room temperature was treated with NaClO₂ (9.45 g, 104.47 mmol, 3.7 eq) and NaH₂PO₄ (6.78 g, 56.5 mmol, 2 eq.). The resulting reaction mixture was allowed to stir at room temperature for 16 h, then concentrated under vacuum, poured into water (500 mL), acidified using dil. HCl (pH −6.0) and extracted using ethyl acetate (3×350 mL). The combined organic layer was dried over Na₂SO₄ and concentrated under vacuum; the crude material was purified by column chromatography (product eluted in 30% EtOAc in Hexane) yielding the title compound as an off-white solid (17 g, Yield: 97%).

¹H NMR (DMSO-d6, 400 MHz): δ ppm 1.85 (s, 3H), 2.45 (s, br, 6H), 4.81 (s, 2H), 6.75 (s, 1H), 7.33-7.40 (m, 5H), 7.50-7.53 (m, 4H), 7.72-7.75 (m, 4H), 13.75 (bs, 1H). LCMS (Method A): 2.43 min, MS: ES+582.6 (M+1).

Amine Intermediates

| No | Structure | Name | Data |
|---|---|---|---|
| 9 | 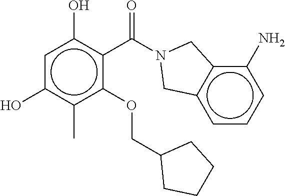 | (4-Aminoisoindolin-2- yl)(2- (cyclopentylmethoxy)- 4,6-dihydro-3- methylphenyl)methanone | Prepared from Intermediate 25 and 4- nitro-isoindoline (CAS: 127168-86-9) according to the method of Intermediate 1. ¹H NMR (DMSO-d6, 400 MHz): δ ppm 1.23-1.31 (m, 2H), 1.44-1.50 (m, 4H), 1.58-1.64 (m, 2H), 1.92 (d, J = 2.8 Hz, 3H), 2.15-2.18 (m, 1H), 3.56-3.70 (m, 2H), 4.32-4.64 (m, 4H), 5.05-5.19 (m, 2H, D₂O exchangeable), 6.23 (d, J = 3.6 Hz, 1H), 6.38-6.53 (m, 2H), 6.91-6.98 (m, 1H), 9.33-9.43 (m, 2H). LCMS (Method A): 1.776 min, MS ES+: 383.06 |
| | | | (M + 1). |
| 10 | 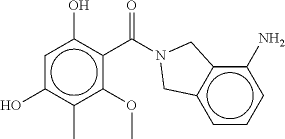 | (4-Aminoisoindolin-2- yl)(4,6-dihydroxy-2- methoxy-3- methylphenyl)methanone | Prepared from Intermediate 26 and 4- nitro-isoindoline (CAS: 127168-86-9) according to the method of Intermediate 1. ¹H NMR (DMSO-d6, 400 MHz): δ ppm 1.92 (d, J = 2.8 Hz, 3H), 3.61 (d, J = 1.6 Hz, 3H), 4.26-4.65 (m, 4H), 5.05 (s, 1H), 5.17 (s, 1H), 6.25 (d, J = 5.5 Hz, 1H), 6.38-6.52 (m, 2H), 6.91-6.98 (m, 1H), 9.39-9.46 (dd, J = 5.6 Hz, 4.8 Hz, 2H). LCMS |
| | | | (Method A): 1.187 min, MS ES+: 314.89 |
| | | | (M + 1), 100%. |
| 11 | 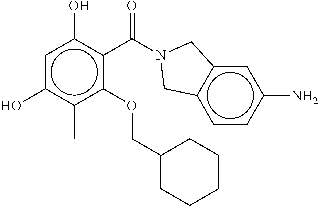 | (5-Aminoisoindolin-2- yl)(2- (cyclohexylmethoxy)-4,6- dihydroxy-3- methylphenyl)methanone | Prepared from Intermediate 5 using 5- nitro-isoindoline (CAS: 46053-72-9) according to the method of Intermediate 1. ¹H NMR (DMSO-d6, 400 MHz): δ ppm 1.05-1.23 (m, 5H), 1.55-1.65 (m, 7H), 1.90 (d, J = 2 Hz, 3H), 3.33-3.39 (m, 1H), 3.61-3.65 (m, 1H), 4.31-4.35 (m, 2H), 4.49-4.58 (m, 2H), 5.04 (d, J = 11.2 Hz, 2H), 6.22 (d, J = 3.2 Hz, 1H), 6.39-6.52 (m, 2H), 6.86-7.00 (m, 1H), 9.31 (d, J = 7.2 Hz, 1H), 9.41 (d, J = 5.2 Hz, 1H). LCMS (Method A): 1.492 min, MS: ES+ 397.01 (M + 1); 100%. |
| 12 | 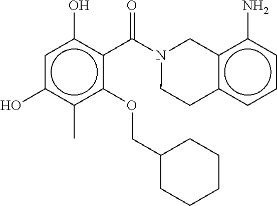 | (8-Amino-3,4- dihydroisoquinolin-2(1H)- yl)(2- (cyclohexylmethoxy)-4,6- dihydroxy-3- methylphenyl)methanone | Prepared from Intermediate 5 using (9H- fluoren-9-yl)methyl(3,4-dihydroxy-1H- isoquinolin-8-yl)carbamate hydrochloride (Intermediate 30) followed by removal of protecting groups according to the method described from Intermediate 1. ¹H NMR (DMSO-d6, 400 MHz, 344K): δ ppm 0.92-0.97 (m, 2H), 1.06-1.17 (m, 3H), 1.56-1.64 (m, 6H), 1.94 (s, 3H), 2.70- 2.72 (m, 2H), 3.46-3.59 (m, 4H), 4.41- 4.73 (m, 4H), 6.21 (s, 1H), 6.37 (d, J = 6.4 Hz, 1H), 6.53 (d, J = 8.0 Hz, 1H), 6.86 (t, J = 7.6 Hz, 1H), 8.96 (s, 1H), 9.11 (s, 1H). |
| | | | LCMS (Method A): 1.932 min, MS ES+: |
| | | | 411 (M + 1), 98.7%. |
| 13 | 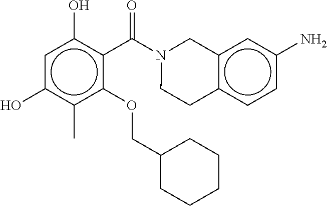 | 7-Amino-3,4-dihydro- isoquinolin-2(1H)-yl)(2- (cyclohexylmethoxy)-4,6- dihydroxy-3- methylphenyl)methanone | Prepared from Intermediate 5 using 7- nitro-1,2,3,4-tetrahydroisoquinoline (CAS 42923-79-5) according to the method described for Intermediate 1. ¹H NMR (DMSO-d6, 400 MHz): δ ppm 0.78-0.94 (m, 2H), 1.06-1.16 (m, 3H), 1.46-1.58 (m, 6H), 1.91 (s, 3H), 2.55-2.57 (m, 2H), 2.64-2.67 (m, 1H), 3.37-3.58 (m, 2H), 4.21 (s, 1H), 4.55 (d, J = 4.4 Hz, 1H), 4.80- 4.88 (m, 2H), 6.13-6.20 (m, 1H), 6.37- 6.40 (m, 2H), 6.75-6.80 (m, 1H), 9.24 (s, 1H), 9.39 (d, J = 7.2 Hz, 1H). LCMS (Method A): 1.479 min, MS ES+: 411.16 |
| | | | (M + 1), 99.6%. |
| 14 | 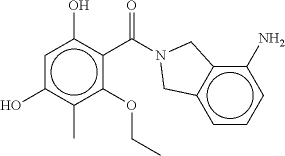 | (4-Aminoisoindolin-2- yl)(2-ethoxy-4,6- dihydroxy-3- methylphenyl)methanone | Prepared from Intermediate 27 and 4- nitro-isoindoline (CAS: 127168-86-9) according to the method described for Intermediate 1. ¹H NMR (DMSO-d6, 400 MHz): δ ppm 1.18 (t, J = 6.8 Hz, 3H), 1.92 (d, J = 2.4 Hz, 3H), 3.77-3.87 (m, 2H), 4.26-4.65 (m, 4H), 5.05-5.17 (m, 2H), 6.23 (d, J = 4.0 Hz, 1H), 6.38-6.52 (m, 2H), 6.91-6.98 (m, 1H), 9.35 (d, J = 5.6 Hz, 1H), 9.42 (d, J = 7.2 Hz, 1H). LCMS |
| | | | (Method A): 1.315 min, MS: ES+ 329 |
| | | | (M + 1), 100%. |
| 15 | 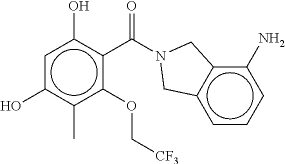 | (4-Aminoisoindolin-2- yl)(4,6-dihydroxy-3- methyl-2-(2,2,2- trifluoroethoxy)phenyl)meth- anone | Prepared from Intermediate 28 and 4- nitro-isoindoline (CAS: 127168-86-9) according to the method described for Intermediate 1. ¹H NMR (DMSO-d6, 400 MHz): δ ppm 1.94 (d, J = 2.4 Hz, 3H), 4.29- 4.39 (m, 2H), 4.45-4.54 (m, 2H), 4.65 (s, 1H), 5.06-5.19 (m, 2H), 6.33 (d, J = 4.8 Hz, 1H), 6.38-6.53 (m, 2H), 6.94-6.97 (m, 1H), 9.61-9.68 (m, 2H). LCMS (Method A): 1.565 min, MS ES+: 383.0 (M + 1), 95.6%. |
| 16 | 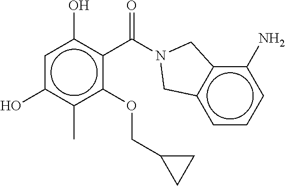 | (4-Aminoisoindolin-2- yl)(2- (cyclopropylmethoxy)- 4,6-dihydroxy-3- methylphenyl)methanone | Prepared from Intermediate 29 and 4- nitro-isoindoline (CAS: 127168-86-9) according to the method described for Intermediate 1. ¹H NMR (DMSO-d6, 400 MHz): δ ppm 0.18-0.20 (m, 2H), 0.44- 0.46 (m, 2H), 1.05-1.08 (s, br, 1H), 1.93 (s, 3H), 3.50-3.64 (m, 2H), 4.27-4.45 (m, 2H), 4.53 (s, 1H), 4.65 (s, 1H), 5.05 (s, 1H) 5.18 (s, 1H), 6.24 (d, J = 4 Hz, 1H), 6.39-6.53 (m, 2H), 6.92-6.99 (m, 1H), 9.34-9.43 (m, 2H). LCMS (Method A): 1.453 min, MS ES+: 355 (M + 1), 97.6%. |
| 18 | 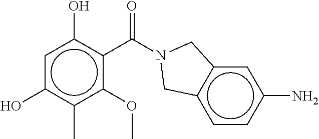 | (5-Aminoisoindolin-2- yl)(4,6-dihydroxy-2- methoxy-3- methylphenyl)methanone | Prepared from Intermediate 26 and 5- nitro-isoindoline (CAS: 46053-72-9) according to the method of Intermediate 1. ¹H NMR (DMSO-d6, 400 MHz): δ ppm 1.92 (d, J = 1.6 Hz, 3H), 3.60 (s, 3H), 4.24- 4.38 (m, 2H), 4.58 (d, J = 12 Hz, 2H), 5.05 (d, J = 12.8 Hz, 2H), 6.24 (d, J = 3.2 Hz, 1H), 6.39 (s, 1H), 6.45-6.52 (m, 2H), 6.86- 7.00 (m, 1H), 9.38 (d, J = 8 Hz, 1H), 9.45 |
| | | | (d, J = 5.2 Hz, 1H). LCMS (Method A): |
| | | | 0.862 min, MS: ES+ 314.8 (M + 1), 97%. |
| 20 | 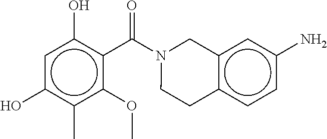 | (7-Amino-3,4- dihydroisoquinolin-2(1H)- yl)(4,6-dihydroxy-2- methoxy-3- methylphenyl)methanone | Prepared from Intermediate 26 and 7- nitro-1,2,3,4-tetrahydroisoquinoline (CAS: 42923-79-5) according to the method of Intermediate 1. ¹H NMR (DMSO-d6, 400 MHz, 348K): δ ppm 1.92 (s, 3H), 2.59- 3.08 (m, 2H), 3.57 (s, 4H), 4.46-4.65 (m, br, 4H), 6.22 (s, 1H), 6.34-6.43 (m, 2H), 6.79 (d, J = 8 Hz, 1H), 8.99 (s, 1H), 9.13 (s, 1H). LCMS (Method A): 0.934 min, MS |
| | | | ES+: 328.8 (M + 1), 100%. |
| 21 | 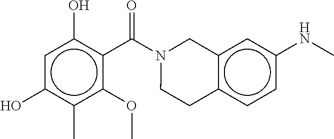 | (4,6-Dihydroxy-2- methoxy-3- methylphenyl)(7- (methylamino)-3,4- dihydroisoquinolin-2(1H)- yl)methanone | Prepared from Intermediate 26 and 7- nitro-1,2,3,4-tetrahydroisoquinoline (CAS: 42923-79-5) according to the method of Intermediate 19. ¹H NMR (DMSO-d6, 400 MHz, 348K): δ ppm 1.91 (d, J = 6.8 Hz, 3H), 2.56-2.65 (m, br, 5H), 3.50-3.54 (m, 5H), 4.26-4.29 (m, 1H), 4.61-4.64 (m, 1H), 5.47-5.49 (m, 1H, D₂O exchangeable), 6.10-6.22 (m, 1H), 6.35- |
| | | | 6.40 (m, 2H), 6.82-6.87 (m, 1H), 9.31- |
| | | | 9.33 (m, 1H), 9.45 (s, 1H). LCMS (Method |
| | | | A): 1.001 min, MS ES+: 343.1 (M + 1), |
| | | | 94.5%. |
| 22 | 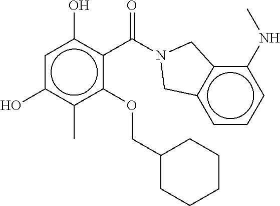 | (2-(Cyclohexylmethoxy)- 4,6-dihydroxy-3- methylphenyl)(4- (methylamino)isoindolin- 2-yl)methanone | Prepared from Intermediate 5 and 4-nitro- isoindoline (CAS: 127168-86-9) according to the method of Intermediate 19. ¹H NMR (DMSO-d6, 400 MHz, 348K): δ ppm 0.90-1.09 (m, 2H), 1.10-1.27 (m, 4H), 1.57-1.69 (m, 6H), 1.96 (s, 3H), 2.66 and 2.76 (2 singlets, 3H), 3.56-3.68 (m, 2H), 4.36 (bs, 1H), 4.46 (bs, 1H), 4.57 (bs, 1H), 4.66-4.69 (m, 1H), 5.13-5.28 (m, 1H, D₂O exchangeable), 6.24 (s, 1H), 6.37-6.59 (m, 2H), 7.08-7.11 (m, 1H), 9.02 (bs, 1H), 9.13 (bs, 1H). LCMS (Method A): 2.156 min, MS: ES+ 411.2 |
| | | | (M + 1), 100%. |
| 23 | 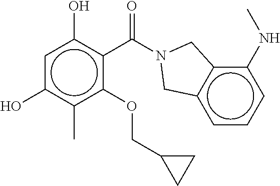 | (2-(cyclopropylmethoxy)- 4,6-dihydroxy-3- methylphenyl)(4- (methylamino)isoindolin- 2-yl)methanone | Prepared from Intermediate 29 and 4- nitro-isoindoline (CAS: 127168-86-9) according to the method of Intermediate 19. ¹H NMR (DMSO-d6, 400 MHz, 348K): δ ppm 0.21 (d, J = 3.6 Hz, 2H), 0.44-0.46 (m, 2H), 1.06-1.13 (m, 1H), 1.97 (s, 3H), 2.66-2.77 (2 singlets, 3H), 3.64-3.66 (m, 2H), 4.35 (bs, 1H), 4.46 (bs, 1H), 4.69 (bs, 1H), 4.70 (bs, 1H), 5.13-5.27 (m, 1H, D₂O exchangeable), 6.24 (s, 1H), 6.38-6.47 (m, 1H), 6.58 (d, J = 6.8 Hz, 1H), 7.09- 7.13 (m, 1H), 9.04 (bs, 2H), 9.13 (bs, 1H). |
| | | | LCMS (Method A): 1.700 min, MS ES+: |
| | | | 369.15 (M + 1), 99.3%. |
| 24 | 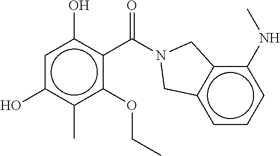 | (2-Ethoxy-4,6-dihydroxy- 3-methylphenyl)(4- (methylamino)isoindolin- 2-yl)methanone | Prepared from Intermediate 27 and N- methylisoindolin-4-amine hydrochloride (Intermediate 31). ¹H NMR (DMSO-d6, 400 MHz, D₂O exchange): δ ppm 1.17 (t, J = 6.8 Hz, 3H), 1.91 (s, 3H), 2.60 and 2.70 (2 singlets, 3H), 3.39-3.84 (m, 2H), 4.28-4.39 (m, 2H), 4.55 (s, 1H), 4.66 (s, 1H), 6.35 (s, 1H), 6.37-6.58 (m, 2H), 7.08- 7.11 (m, 1H). LCMS (Method A): 1.590 min, MS: ES+ 343.0 (M + 1), 100%. |

Acid Intermediates

| No | Structure | Name | Data |
|---|---|---|---|
| 25 | 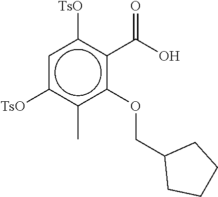 | 2-(Cyclopentylmethoxy)- 3-methyl-4,6- bis(tosyloxy)benzoic acid | Prepared from Intermediate 8 according to the method described for Intermediate 6. ¹H NMR (DMSO-d6, 400 MHz): δ ppm 1.48-1.72 (m, 8H), 1.81 (s, 3H), 2.15- 2.19 (m, 1H), 2.44 (s, 6H), 3.64 (d, J = 11.6 Hz, 2H), 6.70 (s, 1H), 7.48-7.51 (m, 4H), 7.70-7.73 (m, 4H), 13.61 (bs, 1H). LCMS (Method A): 2.589 min, 2.707 min, MS: ES+ 574.99 (M + 1). |
| 26 | 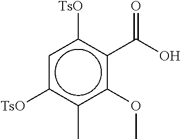 | 2-Methoxy-3-methyl-4,6- bis(tosyloxy)benzoic acid | Prepared from Intermediate 8 according to the method described fro Intermediate 6. ¹H NMR (DMSO-d6, 400 MHz): δ ppm. 1.85 (s, 3H), 2.45 (s, 6H), 3.64 (s, 3H), 6.66 (s, 1H), 7.49-7.52 (m, 4H), 7.72- 7.73 (m, 4H), 13.60 (bs, 1H). LCMS (Method A): 2.137 min, MS ES+ 507.02 (M + 1). |
| 27 | 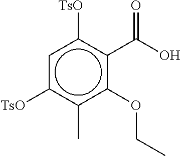 | 2-Ethoxy-3-methyl-4,6- bis(tosyloxy)benzoic acid | Prepared from Intermediate 8 according to the method described for Intermediate 6. ¹H NMR (DMSO-d6, 400 MHz): δ ppm. 1.17-(t, J = 6.8 Hz, 3H), 1.83 (s, 3H), 2.45 (s, 6H), 3.78-3.83 (q, J = 6.8 Hz, 2H), 6.68 (s, 1H), 7.49-7.58 (m, 4H), 7.71-7.80 (m, 4H), 1.359 (s, 1H). LCMS (Method A): 2.459 min, 2.650 min (Two peak mass observed) MS ES+ 521.12 (M + 1). |
| 28 | 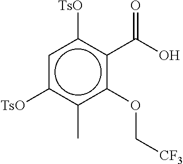 | 3-Methyl-4,6- bis(tosyloxy)-2-(2,2,2- trifluoroethoxy)benzoic acid | Prepared from Intermediate 8 according to the method described for Intermediate 6. ¹H NMR (DMSO-d6, 400 MHz): δ ppm. 1.82 (s, 3H), 2.43 (d, J = 4.4 Hz, 6H), 4.55 (dd, J = 8.4 Hz, 2H), 6.78 (s, 1H), 7.48 (t, J = 8 Hz, 4H), 7.69 (d, J = 8.8 H, 2H), 7.78 (d, J = 7.6 Hz, 2H), 14.08 (bs, 1H). LCMS (Method A): 2.357 min, MS: ES+ 574.8 (M + 1), 99.4%. |
| 29 | 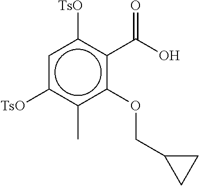 | 2-(Cyclopropylmethoxy)- 3-methyl-4,6- bis(tosyloxy)benzoic acid | Prepared from Intermediate 8 according to the method described for Intermediate 6. ¹H NMR (DMSO-d6, 400 MHz): δ ppm. 0.12-0.16 (m, 2H), 0.44-0.47 (m, 2H), 1.00-1.10 (m, 1H), 1.83 (s, 3H), 2.43 (s, 6H), 3.58-3.64 (m, 2H), 6.69 (s, 1H), 7.48- 7.51 (m, 4H), 7.70-7.77 (m, 4H), 13.58 (bs, 1H). LCMS (Method A): 2.418 min, 2.453 min, MS ES+ 569.2 (M + 23). |

### 4,6-Dihydroxy-2-methoxy-3-methylphenyl) (4-(methylamino)isoindolin-2-yl) methanone: Intermediate 17

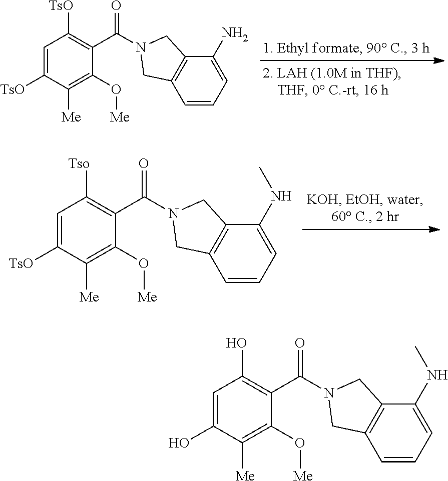

A stirred solution of 4-(4-aminoisoindoline-2-carbonyl)-5-methoxy-6-methyl-1,3-phenylene bis(4-methyl-benzenesulfonate) [prepared from Intermediate 26 and 4-nitroisoindoline (CAS: 127168-86-9) using methods described for Intermediate 1] (0.8 g, 1.284 mmol, 1.0 eq.) in ethyl formate (8 mL) was heated to 90° C. and stirred for 3 h, cooled and concentrated under vacuum. Crude material was dissolved in THF (8 mL), cooled to 0° C. and treated with LiAlH₄ (1.0 M in THF) (1.92 mL, 1.92 mmol, 1.5 eq.) added dropwise at 0° C. The resulting reaction mixture was stirred at room temperature for 16 h and then poured onto ice-cold water (100 mL) followed by addition of ethyl acetate (100 mL). The reaction mixture was filtered through a celite bed and washed with ethyl acetate (100 mL). The organic layer was separated and dried over Na₂SO₄, filtered, and concentrated under vacuum. Crude material was purified by flash chromatography (product eluted with 3% MeOH in DCM) yielding the title compound (0.45 g, Yield: 55%). LCMS (Method A): 2.537 min, 2.628 min, MS ES+: 636.95 (M+1).

A stirred solution of 5-methoxy-4-methyl-6-(4-(methylamino)isoindoline-2-carbonyl)-1,3-phenylene bis(4-methylbenzenesulfonate) (0.45 g, 0.706 mmol, 1.0 eq.) in EtOH: H₂O (7:3) (18 mL) at room temperature was treated with KOH (1.58 g, 28.26 mmol, 40 eq.). The reaction mixture was heated to 60° C. and stirred for 2 h, cooled and concentrated under vacuum. Water (20 mL) was added to crude material and acidified using saturated aq. KHSO₄ at 0° C. The aqueous layer was extracted using ethyl acetate (2×250 mL) and the combined organics dried over Na₂SO₄, filtered and concentrated under vacuum. Crude material was purified by flash chromatography (product eluted with 5% MeOH in DCM) yielding the title compound as an off white solid (0.130 g, Yield: 56%).

High temperature ¹H NMR (DMSO-d6, 400 MHz, 349K): δ ppm 1.96 (s, 3H), 2.67 and 2.77 (2 singlets, 3H), 3.64 (s, 3H), 4.35-4.70 (m, 4H), 5.11-5.23 (m, 1H), 6.26 (s, 1H), 6.40-6.57 (m, 2H), 7.10 (s, 1H), 9.08 (s, 1H) 9.15 (s, 1H). LCMS (Method A): 1.436 min, MS ES+: 328.95 (M+1).

### (4,6-Dihydroxy-2-methoxy-3-methylphenyl)(5-(methylamino)isoindolin-2-yl)methanone: Intermediate 19

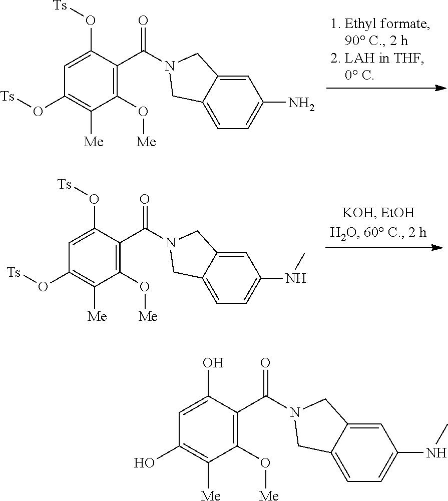 [SMILES: COc1c(C)c(OS(=O)(=O)c2ccc(C)cc2)cc(OS(=O)(=O)c2ccc(C)cc2)c1C(=O)N1Cc2ccc(N)cc2C1 ; CNc1ccc2c(c1)CN(C(=O)c1c(OS(=O)(=O)c3ccc(C)cc3)cc(OS(=O)(=O)c3ccc(C)cc3)c(C)c1OC)C2 ; CNc1ccc2c(c1)CN(C(=O)c1c(O)cc(O)c(C)c1OC)C2]

A stirred solution of 4-(5-aminoisoindoline-2-carbonyl)-5-methoxy-6-methyl-1,3-phenylene bis(4-methylbenzenesulfonate) [prepared from Intermediate 26 and 5-nitroisoindoline (CAS: 46053-72-9) using methods described for Intermediate 1] (0.8 g, 1.28 mmol, 1.0 eq.) in ethyl formate (8 mL) was heated to 90° C. and stirred for 2 h, cooled and concentrated under vacuum. The crude material was dissolved in THF (8 mL) and cooled to 0° C. and treated with LAlH₄ (1.0 M in THF) (6.43 mL, 1.28 mmol, 5.0 eq.) added dropwise at 0° C. The resulting reaction mixture was stirred at room temperature for 4 h, poured into ice cold NH₄Cl solution and extracted using EtOAc (3×20 mL). The combined organic layer was dried over Na₂SO₄, filtered, and concentrated under vacuum yielding 5-methoxy-4-methyl-6-(5-(methylamino)isoindoline-2-carbonyl)-1,3-phenylene bis(4-methylbenzenesulfonate) (0.9 g, Yield: 55%, 1.41 mmol). The crude material was used in the next step without purification.

A stirred solution of 5-methoxy-4-methyl-6-(5-(methylamino)isoindoline-2-carbonyl)-1,3-phenylene bis(4-methylbenzenesulfonate) (0.9 g, 1.86 mmol, 1.0 eq.) in EtOH:H₂O (1:1) (13.5 mL) at room temperature was treated with aq. KOH (4.1 g, 74.7 mmol, 40 eq.). The reaction mixture was heated to 60° C. and stirred for 1 h, cooled, and concentrated under reduced pressure. The obtained material was acidified using KHSO₄ and then neutralized with saturated NaHCO₃. The aqueous layer was further extracted with 10% MeOH in DCM (5×50 mL). The combined organic layer was dried over Na₂SO₄, filtered, and concentrated under reduced pressure. Crude material was purified by flash chromatography (product eluted with 2.5% methanol in dichloromethane) yielding (4,6-dihydroxy-2-methoxy-3-methylphenyl)(5-(methylamino)isoindolin-2-yl)methanone (Intermediate 19) (0.147 g, Yield: 32%).

¹H NMR (DMSO-d6, 400 MHz): δ ppm 1.92 (s, 3H), 2.61-2.90 (m, 3H), 3.63 (s, 3H), 4.35-4.43 (m, 2H), 4.59-4.67 (m, 2H), 6.24 (d, J=3.2 Hz, 1H), 6.45-6.55 (m, 2H), 6.93-7.07 (m, 1H), 9.38 (d, J=8.4 Hz, 1H), 9.44-9.54 (m, 1H). LCMS (Method A): 0.985 min, MS: ES+328.8 (M+1).

### (9H-Fluoren-9-yl)methyl(1,2,3,4-tetrahydroisoquinolin-8-yl)carbamate hydrochloride: Intermediate 30

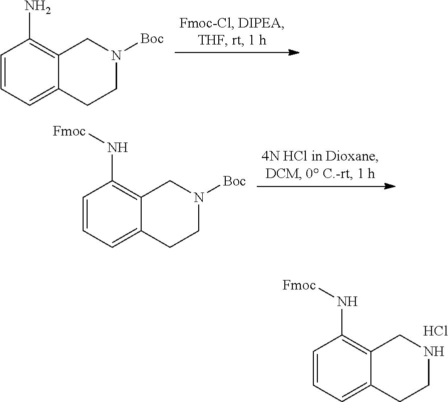

A solution of t-butyl 8-amino-3,4-dihydroisoquinoline-2(1H)-carboxylate (CAS 910442-87-4) (1.0 g, 4.0 mmol, 1 eq.) in THF at room temperature was treated with Fmoc-CI (1.04 g, 4.0 mmol, 1 eq.) and DIPEA (1.56 g, 12.09 mmol, 3 eq.) and stirred for 1 h; the reaction mixture was poured into water (200 mL) and extracted using ethyl acetate (3×100 mL). The combined organic layer was dried over Na₂SO₄, filtered and concentrated under vacuum. Crude material was purified by flash chromatography (product eluted in 9% EtOAc in Hexane) yielding tert-butyl 8-((((9H-fluoren-9-yl)methoxy)carbonyl)amino)-3,4-dihydroisoquinoline-2(1H)-carboxylate as white solid (2 g, Yield: Quantitative, 4.25 mmol).

¹H NMR (DMSO-d6, 400 MHz): δ ppm 1.44 (s, 9H), 2.78 (t, J=5.6 Hz, 2H), 3.51 (s, 2H), 4.29-4.31 (m, 1H), 4.42-4.44 (m, 4H), 7.01-7.17 (m, 3H), 7.34-7.36 (m, 2H), 7.41-7.45 (m, 2H), 7.71 (bs, 2H), 7.92 (d, J=7.2 Hz, 2H), 9.12 (bs, 1H). LCMS (Method A): 2.597 min, MS: ES+370.95 (M−100).

A solution of tert-butyl 8-((((9H-fluoren-9-yl)methoxy)carbonyl)amino)-3,4-dihydroisoquinoline-2(1H)-carboxylate (2 g, 4.2 mmol, 1.0 eq.) in DCM (20 mL) was cooled to 0° C. 4M HCl in dioxane (10 mL) was added dropwise to the reaction mixture at 0° C. The reaction mixture was stirred at room temperature for 1 h, then concentrated under vacuum. Crude material was triturated using diethyl ether (3×25 mL) followed by n-pentane (3×25 mL). The solid material was further dried under high vacuum yielding (9H-fluoren-9-yl)methyl (1,2,3,4-tetrahydroisoquinolin-8-yl)carbamate hydrochloride as an off white solid (1.5 g, Yield: 87.23%, 4.0 mmol).

¹H NMR (DMSO-d6, 400 MHz): δ ppm 3.01 (t, J=6 Hz, 2H), 3.30-3.31 (m, 2H), 3.57 (s, 3H), 4.10-4.11 (m, 2H), 4.31 (t, J=6.4 Hz, 1H), 4.48 (d, J=6.8 Hz, 2H), 7.06-7.26 (m, 3H), 7.35-7.46 (m, 4H), 7.73 (d, J=6.4 Hz, 2H), 7.92 (d, J=7.2 Hz, 2H), 9.25 (bs, 1H), 9.44 (s, 1H). LCMS (Method A): 1.519 min, MS: ES+371.15 (M+1).

### N-Methylisoindolin-4-amine hydrochloride: Intermediate 31

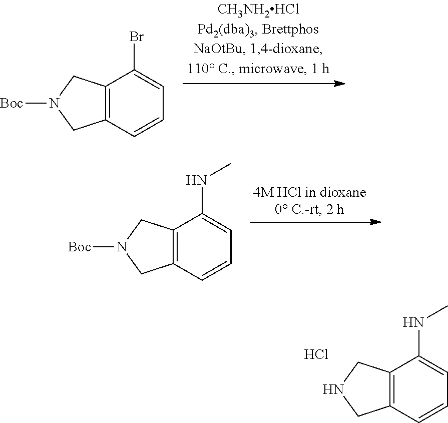 [SMILES: CC(C)(C)OC(=O)N1Cc2cccc(Br)c2C1 ; CNc1cccc2c1CN(C(=O)OC(C)(C)C)C2 ; Cl ; CNc1cccc2c1CNC2]

A stirred solution of t-butyl 4-bromoisoindoline-2-carboxylate (CAS 1035235-27-8) (1.0 g, 1.0 eq.) and methylamine HCl salt (0.33 g, 1.5 eq.) in 1,4-dioxane (5 mL) at room temperature under nitrogen atmosphere was treated with NaOtBu (0.63 g, 2.0 eq.). The reaction mixture was degassed using N₂ (for 10 minutes. Brettphos (0.10 g, 0.06 eq.) and Pd₂ (dba) 3 (0.090 g, 0.03 eq.) were added to the reaction mixture at room temperature. The reaction mixture was heated to 110° C. under microwave irradiation for 1 h. The reaction mixture was cooled, poured into water (50 mL) and extracted by ethyl acetate (2×100 mL). The combined organic layer dried by Na₂SO₄, filtered, and concentrated under vacuum. Crude material was purified by flash chromatography (product eluted with 10% EtOAc in Hexane) yielding t-butyl 4-(methylamino) isoindoline-2-carboxylate (0.15 g, Yield: 18%).

¹H NMR (DMSO-d6, 400 MHz): 1.45 (s, 9H), 2.68 (s, 3H), 4.36 (d, J=10 Hz, 2H), 4.49 (d, J=9.6 Hz, 2H), 5.39-5.40 (m, 1H), 6.36 (d, J=8 Hz, 1H), 6.49-6.54 (m, 1H), 7.08 (t, J=8 Hz, 1H). LCMS (Method A): 2.055 min, MS: ES+193.18 (M-56).

A stirred solution of t-butyl 4-(methylamino)isoindoline-2-carboxylate (0.15 g, 1.0 eq.) in DCM (2 mL) was cooled to 0° C. 4M HCl in 1,4-dioxane (1 mL) was added to the reaction mixture at 0° C. The resulting reaction mixture was stirred at room temperature for 2 h. The reaction mixture was concentrated under vacuum and the obtained crude triturated using diethyl ether (2×10 mL) followed by drying under high vacuum yielding N-methylisoindolin-4-amine hydrochloride (0.12 g, Yield: quantitative).

¹H NMR (DMSO-d6, 400 MHz): 2.74 (s, 3H), 4.35-4.43 (m, 4H), 6.62-6.73 (m, 2H), 7.22 (t, J=7.6 Hz, 1H), 9.94 (bs, 2H), HCl salt. LCMS (Method A): 0.674 min, MS: ES+149.11 (M+1).

### Biological Assays

### Fluorescence Polarisation Assay for PMS2

Test compounds, as 10 mM DMSO stocks, were dispensed into a Black Fluotrac 200 384 well medium binding plate (Greiner Bio-One, item number 781076) using a Labcyte Echo acoustic liquid handler. For single point screening, test compounds were added to wells in columns 1-22 whilst DMSO was added to wells in columns 23 and 24 in order to normalise the plate. For potency determination, serial dilutions of test compounds were added to wells in columns 3-22 and DMSO volume was normalised across the plate.

20 μL of a 2× solution (20 nM) of recombinant N-terminal PMS2 (residues 1-365) in assay buffer (25 mM HEPES, pH 7.5, 250 mM NaCl, 10 mM MgCl2, 0.01% Triton X-100, 5 mM Dithiothreitol) was added to all wells in columns 2-23 for potency determination or columns 1-23 for single point screening. 20 μL assay buffer was added to all wells in columns 1 and 24 (column 24 only for single point screening) using a MultiDrop Combi (ThermoFisher). Plates were centrifuged for 1 minute at 250×g and were incubated at room temperature for 30 minutes prior to the addition of 20 μL of 2× (20 nM) of 5-((5-(4-((2-(2,4-dihydroxy-5-isopropylbenzoyl)isoindolin5-yl)methyl)piperazin-1-yl)pentyl)carbamoyl)-2-(6-(dimethylamino)-3-(dimethyliminio)-3H-xanthen-9-yl)benzoate (referred to hereinafter as “probe compound”) in assay buffer (prepared from a 100 μM DMSO stock) with a MultiDrop Combi (ThermoFisher). The final concentration of N-terminal PMS2 was 10 nM and the final concentration of probe compound was 5 nM.

Compound plates were centrifuged for 1 minute at 250×g for 1 minute and were incubated at room temperature for 1 hour before being read on a PheraStar FSX (fitted with 384-well aperture spoon and 540 590 590 FP optic module). The gain and focus were adjusted before each plate was read so that the polarisation of a no enzyme control (column 24) was equal to 35 mP. Data were normalised against the no inhibitor controls (column 23) and no enzyme controls (column 24).

Data obtained in this assay is shown in Table A₁ shown below

TABLE A1

| Example | | PMS2 |
|---|---|---|
| No | Name | IC₅₀ |
| 1 | N-(2-(2-(Benzyloxy)-4,6-dihydroxybenzoyl)isoindolin-4- | ** |
| | yl)acrylamide; | |
| 2 | (E)-N-(2-(2-(Benzyloxy)-4,6- | *** |
| | dihydroxybenzoyl)isoindolin-4-yl)-4-(dimethylamino)but- | |
| | 2-enamide; | |
| 3 | N-(2-(2-(Benzyloxy)-4,6-dihydroxy-benzoyl)isoindolin-4- | * |
| | yl)-2-fluoroacrylamide | |
| 4 | N-(2-(2-(Benzyloxy)-4,6-dihydroxy-3- | *** |
| | methylbenzoyl)isoindolin-4-yl)acrylamide | |
| 5 | N-(2-(2-(Benzyloxy)-4,6-dihydroxy-3- | * |
| | methylbenzoyl)isoindolin-4-yl)-2-fluoroacrylamide | |
| 6 | (E)-N-(2-(2-(Benzyloxy)-4,6-dihydroxy-3- | *** |
| | methylbenzoyl)isoindolin-4-yl)-4-(dimethylamino)but-2- | |
| | enamide | |
| 7 | N-(2-(2-(cyclohexylmethoxy)-4,6-dihydroxy-3- | *** |
| | methylbenzoyl) isoindolin-4-yl) acrylamide | |
| 8 | (E)-N-(2-(2-(cyclohexylmethoxy)-4,6-dihydroxy-3- | *** |
| | methylbenzoyl)isoindolin-4-yl)-4-(dimethylamino)but-2- | |
| | enamide | |
| 9 | (E)-N-(2-(2-(Cyclohexylmethoxy)-4,6-dihydroxy-3- | *** |
| | methylbenzoyl)isoindolin-4-yl)-4-(pyrrolidin-1-yl)but-2- | |
| | enamide | |
| 10 | (E)-N-(2-(2-(cyclohexylmethoxy)-4,6-dihydroxy-3- | *** |
| | methylbenzoyl)isoindolin-4-yl)-4-morpholinobut-2- | |
| | enamide hydrochloride | |
| 11 | (E)-N-(2-(2-(Cyclohexylmethoxy)-4,6-dihydroxy-3- | *** |
| | methylbenzoyl)isoindolin-4-yl)-4-(4-methylpiperazin-1- | |
| | yl)but-2-enamide | |
| 12 | N-(2-(2-(Cyclohexylmethoxy)-4,6-dihydroxy-3- | ** |
| | methylbenzoyl)isoindolin-4-yl)but-2-ynamide; | |
| 13 | (E)-N-(2-(2-(Cyclohexylmethoxy)-4,6-dihydroxy-3- | * |
| | methylbenzoyl)isoindolin-4-yl)but-2-enamide | |
| 14 | (E)-N-[2-[2-(Cyclopentylmethoxy)-4,6-dihydroxy-3- | *** |
| | methyl-benzoyl]isoindolin-4-yl]-4-(dimethylamino)but-2- | |
| | enamide | |
| 15 | (E)-N-[2-(4,6-Dihydroxy-2-methoxy-3-methyl- | *** |
| | benzoyl)isoindolin-4-yl]-4-(dimethylamino)but-2- | |
| | enamide | |
| 16 | N-[2-[2-(Cyclohexylmethoxy)-4,6-dihydroxy-3-methyl- | *** |
| | benzoyl]isoindolin-5-yl]prop-2-enamide | |
| 17 | N-[2-[2-(Cyclohexylmethoxy)-4,6-dihydroxy-3-methyl- | ** |
| | benzoyl]-3,4-dihydro-1H-isoquinolin-8-yl]prop-2- | |
| | enamide | |
| 18 | N-[2-[2-(Cyclohexylmethoxy)-4,6-dihydroxy-3-methyl- | *** |
| | benzoyl]-3,4-dihydro-1H-isoquinolin-7-yl]prop-2- | |
| | enamide | |
| 19 | (E)-N-[2-[2-(Cyclohexylmethoxy)-4,6-dihydroxy-3- | ** |
| | methyl-benzoyl]isoindolin-5-yl]-4-(dimethylamino)but-2- | |
| | enamide | |
| 20 | (E)-N-[2-[2-(Cyclohexylmethoxy)-4,6-dihydroxy-3- | ** |
| | methyl-benzoyl]-3,4-dihydro-1H-isoquinolin-8-yl]-4- | |
| | (dimethylamino)but-2-enamide | |
| 21 | (E)-N-[2-[2-(Cyclohexylmethoxy)-4,6-dihydroxy-3- | *** |
| | methyl-benzoyl]-3,4-dihydro-1H-isoquinolin-7-yl]-4- | |
| | (dimethylamino)but-2-enamide | |
| 22 | (E)-4-(Dimethylamino)-N-[2-(2-ethoxy-4,6-dihydroxy-3- | *** |
| | methyl-benzoyl)isoindolin-4-yl]but-2-enamide | |
| 23 | (E)-N-[2-[4,6-Dihydroxy-3-methyl-2-(2,2,2- | *** |
| | trifluoroethoxy)benzoyl]isoindolin-4-yl]-4- | |
| | (dimethylamino)but-2-enamide | |
| 24 | (E)-N-[2-[2-(cyclopropylmethoxy)-4,6-dihydroxy-3- | *** |
| | methyl-benzoyl]isoindolin-4-yl]-4-(dimethylamino)but-2- | |
| | enamide | |
| 25 | (E)-N-[2-(4,6-Dihydroxy-2-methoxy-3-methyl- | *** |
| | benzoyl)isoindolin-4-yl]-4-(dimethylamino)-N-methyl-but- | |
| | 2-enamide | |
| 26 | (E)-N-[2-(4,6-Dihydroxy-2-methoxy-3-methyl- | ** |
| | benzoyl)isoindolin-5-yl]-4-(dimethylamino)but-2- | |
| | enamide | |
| 27 | (E)-N-[2-(4,6-dihydroxy-2-methoxy-3-methyl- | ** |
| | benzoyl)isoindolin-5-yl]-4-(dimethylamino)-N-methyl-but- | |
| | 2-enamide | |
| 28 | (E)-N-[2-(4,6-dihydroxy-2-methoxy-3-methyl-benzoyl)- | ** |
| | 3,4-dihydro-1H-isoquinolin-7-yl]-4-(dimethylamino)but-2- | |
| | enamide | |
| 29 | (E)-N-[2-(4,6-dihydroxy-2-methoxy-3-methyl-benzoyl)- | ** |
| | 3,4-dihydro-1H-isoquinolin-7-yl]-4-(dimethylamino)-N- | |
| | methyl-but-2-enamide | |
| 30 | (E)-N-[2-[2-(Cyclohexylmethoxy)-4,6-dihydroxy-3- | *** |
| | methyl-benzoyl]isoindolin-4-yl]-4-(dimethylamino)-N- | |
| | methyl-but-2-enamide | |
| 31 | (E)-N-[2-[2-(Cyclopropylmethoxy)-4,6-dihydroxy-3- | ** |
| | methyl-benzoyl]isoindolin-4-yl]-4-(dimethylamino)-N- | |
| | methyl-but-2-enamide | |
| 32 | N-[2-(4,6-dihydroxy-2-methoxy-3-methyl- | ** |
| | benzoyl)isoindolin-4-yl]-N-methyl-prop-2-enamide | |
| 33 | (E)-4-(Dimethylamino)-N-(2-(2-ethoxy-4,6-dihydroxy-3- | *** |
| | methylbenzoyl)isoindolin-4-yl)-N-methylbut-2-enamide | |
| 34 | (E)-4-[2-(4,6-dihydroxy-2-methoxy-3-methyl- | ** |
| | benzoyl)isoindolin-5-yl]oxy-N, N-dimethyl-but-2-enamide | |
| 35 | (E)-4-((2-(4,6-Dihydroxy-2-methoxy-3- | ** |
| | methylbenzoyl)isoindolin-4-yl)oxy)-N,N-dimethylbut-2- | |
| | enamide | |
| 36 | N-(2-(4,6-Dihydroxy-2-methoxy-3-methylbenzoyl) | * |
| | isoindolin-4-yl)-2-((dimethyl amino)methyl) acrylamide | |
| 37 | N-(2-(4,6-Dihydroxy-2-methoxy-3- | ** |
| | methylbenzoyl)isoindolin-4-yl)-2- | |
| | (morpholinomethyl)acrylamide | |

*>1 μM
**0.1-1 μM
***less than 0.1 μM

1. A compound, or a pharmaceutically acceptable salt, hydrate or solvate thereof, having the structural Formula (II), shown below: 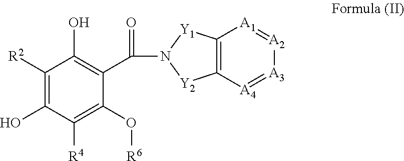
wherein
R² is hydrogen or fluoro;
R⁴ is selected from the group consisting of hydrogen, halogen, (1-6C)alkyl, (1-6C)haloalkyl, (3-6C)cycloalkyl and (3-6C)cycloalkyl(1-2C)alkyl, wherein the said (1-6C)alkyl is optionally substituted by one or more R^{5a} and the said (3-6C)cycloalkyl and (3-6C)cycloalkyl(1-2C)alkyl groups are optionally substituted with one or more R^{5b}; where each R^{5a} is independently selected from halogen or (1-4C)alkoxy and each R^{5b} is independently selected from the group consisting of halogen, (1-4C)alkyl and (1-4C)alkoxy;
R⁶ is (1-6C)alkyl, (1-6C)haloalkyl, (3-8C)cycloalkyl, or a 4- to 7-membered heterocyclyl ring comprising one heteroatom selected from N, O or S,
or a group having a structure according to formula (A) shown below: 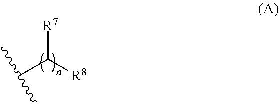
wherein
R⁷ is hydrogen or (1-3C)alkyl;
n is 1 or 2;
R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂—, —C(═O)—, or —CHR_{y2a}—, where R_{y2a} is selected from halo, cyano, methyl, methoxy, CF₃, —OCF₃ or hydroxymethyl;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH, CR¹⁰ or CR¹²;
A₂ is selected from N, CH, CR¹⁰ or CR¹²;
A₃ is selected from N, CH or CR¹³;
A₄ is selected from N, CH or CR¹⁴;
with the proviso that:
only one or two of A₁, A₂, A₃ or A₄ can be N; and
one of A₁ and A₂ is CR¹⁰;
R¹⁰ is a group of the formula (IIA) or (IIB) shown below: 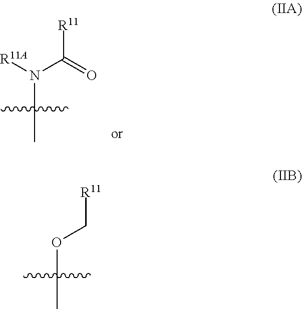
wherein [symbol] denotes the point of attachment to A₁ or A₂,
R^{11A} is hydrogen or (1-3C)alkyl;
R¹¹ is a group of the formula (A) or (B) shown below: 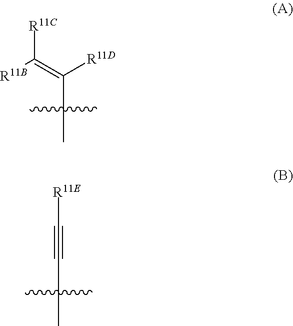
wherein:
R^{11B} and R^{11C} are each independently selected from the group consisting of hydrogen, halo, (1-4C)alkyl, (3-6C)cycloalkyl, (3-6C)cycloalkyl-(1-2C)alkyl and —C(O)NR^{11F}R^{11G} wherein R^{11F} and R^{11G} are each independently selected from hydrogen or (1-4C)alkyl;
and wherein any (1-4C)alkyl, (3-6C)cycloalkyl, (3-6C)cycloalkyl-(1-2C)alkyl present in a R^{11B} and/or R^{11C} group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
R^{11D} is selected from hydrogen, halo, (1-4C)alkyl, (3-6C)cycloalkyl, (3-6C)cycloalkyl-(1-2C)alkyl and —C(O)H, wherein any (1-4C)alkyl, (3-6C)cycloalkyl, (3-6C)cycloalkyl-(1-2C)alkyl or —C(O)H group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy or (1-3C)alkyl;
R^{11E} is selected from the group consisting of hydrogen, halo, (1-4C)alkyl, (3-6C)cycloalkyl, (3-6C)cycloalkyl-(1-2C)alkyl, wherein any (1-4C)alkyl, (3-6C)cycloalkyl or (3-6C)cycloalkyl-(1-2C)alkyl group is optionally substituted with one or more substituents selected from halo, hydroxy, cyano, [(1-4C)alkyl]amino, di-[(1-4C)alkyl]amino, (1-4C)alkoxy, (3-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-4C)alkyl, (1-4C)alkoxy, (3-6C)cycloalkyl, (3-6C)cycloalkoxy;
R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRₚ)_{f}—Z¹²,
wherein Rₚ is hydrogen or methyl;
wherein f is 0 or 1; and
Z¹² is —OR²⁰, —NR²¹R²², —C(O)NR²¹R²² or —NR²³C(O)R²⁴;
wherein R²⁰ is (1-4C)alkyl, (3-7C)cycloalkyl, phenyl, a carbon-linked 4 to 6-membered heterocyclyl, a 5 or 6 membered heteroaryl, —(CHR_{q})ₑ-(3-7C)cycloalkyl, —(CHR_{g})ₑ-phenyl, —(CHR_{q})ₑ-[4 to 6-membered heterocyclyl] or —(CHR_{q})ₑ-[5 or 6 membered heteroaryl], wherein R^{a} is hydrogen or methyl and e is 0 or 1;
R²¹ and R²² are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRᵣ)_{d}-(3-7C)cycloalkyl, —(CHRᵣ)_{d}-phenyl, —(CHRᵣ)_{d}-[4 to 6-membered heterocyclyl] or —(CHRᵣ)_{d}-[⁵ or 6 membered heteroaryl], wherein Rᵣ is hydrogen or methyl and d is 0 or 1;
or R²¹ and R²² are linked, such that, together with the nitrogen atom to which they are attached, they form a 4-6 membered heterocyclic ring;
R²³ is hydrogen or (1-2C)alkyl;
R²⁴ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₛ)_{c}-(3-7C)cycloalkyl, —(CHRₛ)_{c}-phenyl, —(CHRₛ)_{c}-[4 to 6-membered heterocyclyl] or —(CHRₛ)_{c}-[5 or 6 membered heteroaryl], wherein Rₛ is hydrogen or methyl and c is 0 or 1;
wherein each of R²⁰, R²¹, R²², R²³ or R²⁴ or any ring formed when R²¹ and R²² are linked, is optionally substituted with one or more R^{a}; or
R¹³ is selected from cyano, halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein Rₒ is hydrogen or methyl;
wherein h is 0 or 1; and
Z¹³ is —OR²⁵, —NR²⁶R²⁷, —C(O)NR²⁶R²⁷ or —NR²⁸C(O)R²⁹;
wherein R²⁵ is (1-4C)alkyl, (3-7C)cycloalkyl, phenyl, a carbon-linked 4 to 6-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 6-membered heterocyclyl] or —(CHRₙ)ᵢ-[5 or 6 membered heteroaryl], wherein Rₙ is hydrogen or methyl and i is 0 or 1;
R²⁶ and R²⁷ are each independently selected from hydrogen, (1-6C)alkyl, (2-6C)alkanoyl, (3-7C)cycloalkyl, phenyl, a carbon-linked 4 to 6-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[⁴ to 6-membered heterocyclyl] or —(CHRₘ)ⱼ-[⁵ or 6 membered heteroaryl], wherein Rₘ is hydrogen or methyl and j is 0 or 1;
or R²⁶ and R²⁷ are linked, such that, together with the nitrogen atom to which they are attached, they form a 4-6 membered heterocyclic ring;
R²⁸ is hydrogen or (1-2C)alkyl;
R²⁹ is (1-6C)alkyl, (2-6C)alkynyl, (3-7C)cycloalkyl, phenyl, a carbon-linked 4 to 6-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 6-membered heterocyclyl] or —(CHRₗ)ₖ-[5 or 6 membered heteroaryl], wherein Rᵢ is hydrogen or methyl and k is 0 or 1;
wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen or methyl;
wherein m is 0 or 1; and
Z¹⁴ is —OR³⁰, —NR³¹R³², —C(O)NR³¹R³² or —NR³³C(O)R³⁴;
wherein R³⁰ is (1-4C)alkyl, (3-7C)cycloalkyl, a carbon-linked 4 to 6-membered heterocyclyl, a 5 or 6 membered heteroaryl, —(CHRⱼ)ₒ-(3-7C)cycloalkyl, —(CHRⱼ)ₒ-[⁴ to 6-membered heterocyclyl] or —(CHRⱼ)ₒ-[⁵ or 6 membered heteroaryl], wherein R; is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 6-membered heterocyclyl, a 5 or 6 membered heteroaryl, —(CHRᵢ)ₚ-(3-7C)cycloalkyl, —(CHRᵢ)ₚ-[4 to 6-membered heterocyclyl] or —(CHRᵢ)ₚ-[5 or 6 membered heteroaryl], wherein R; is hydrogen or methyl and p is 0 or 1 or R³¹ and R³² are linked, such that, together with the nitrogen atom to which they are attached, they form a 4-6 membered heterocyclic ring;
R³³ is hydrogen or (1-2C)alkyl;
R³⁴ is (1-6C)alkyl, (2-6C)alkynyl, (3-7C)cycloalkyl, a carbon-linked 4 to 6-membered heterocyclyl, a 5 or 6 membered heteroaryl, —(CHRₕ)_{q}-(3-7C)cycloalkyl, —(CHRₕ)_{q}-[4 to 6-membered heterocyclyl] or —(CHRₕ)_{q}-[⁵ or 6 membered heteroaryl], wherein Rₕ is hydrogen or methyl and q is 0 or 1;
wherein R³⁰, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group
-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —N(R^{14a})—C(O)—NR^{14a}—, —SO₂N(R^{14a})—, or —N(R^{14a})SO₂—, where R^{14a} is hydrogen or (1-2C)alkyl; and
Q¹ is selected from the group consisting of hydrogen, (1-4C)alkyl, (2-4C)alkenyl, (2-4C)alkynyl, or (3-6C)cycloalkyl.

2. A compound according to claim 1 or a pharmaceutically acceptable salt, hydrate or solvate thereof, having the structural Formula (I), shown below: 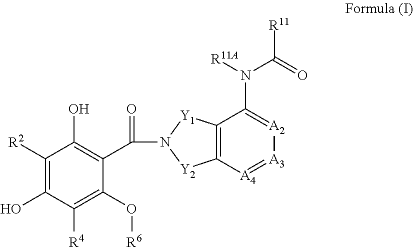
wherein R², R⁴, R⁶, Y₁, Y₂, R^{11A}, R¹¹, A₂, A₃, or A₄ are as defined in claim 1.

3. A compound according to claim 1 or 2, wherein the compound is a compound of the formula I-I, I-II, I-III, I-IV, I-V, I-VI, I-VII, I-VIII, I-IX, I-X, I-XI, I-XII, I-XIII, I-XIV, I-XV, I-XVI, I-XVII and I-XVIII shown below, or pharmaceutically acceptable salt, hydrate or solvate thereof: 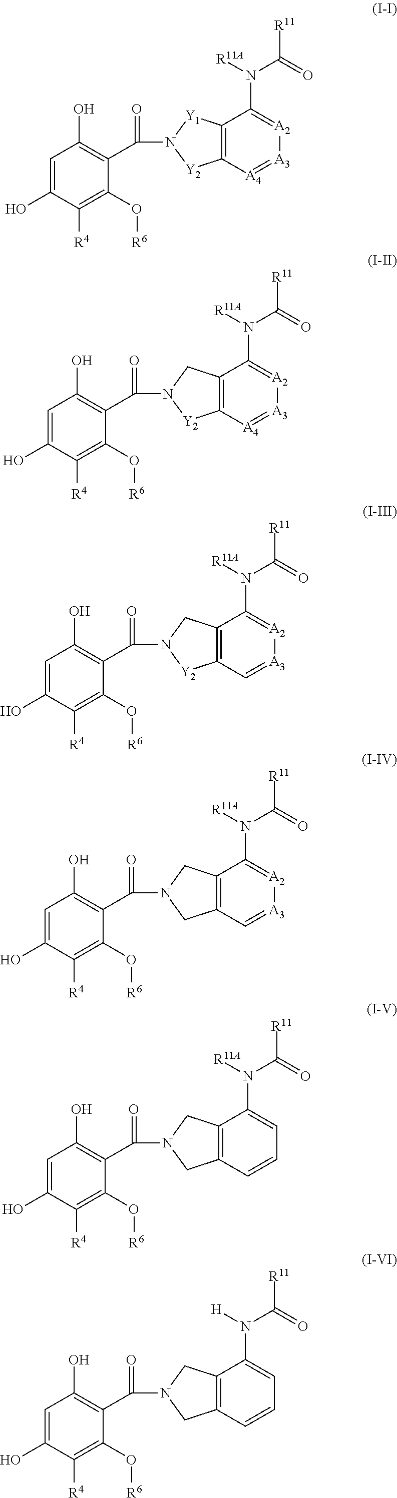 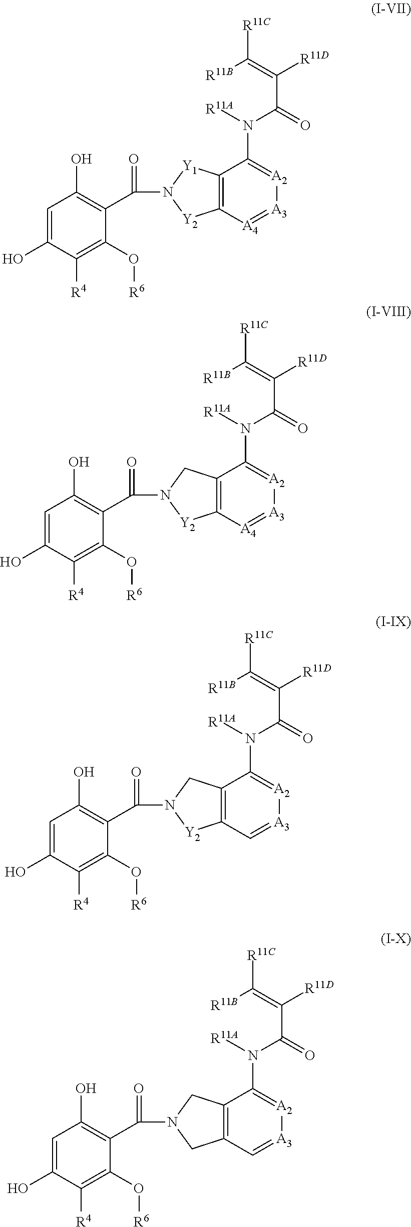 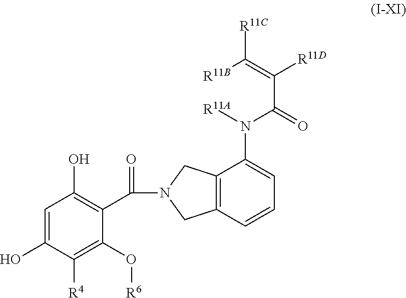 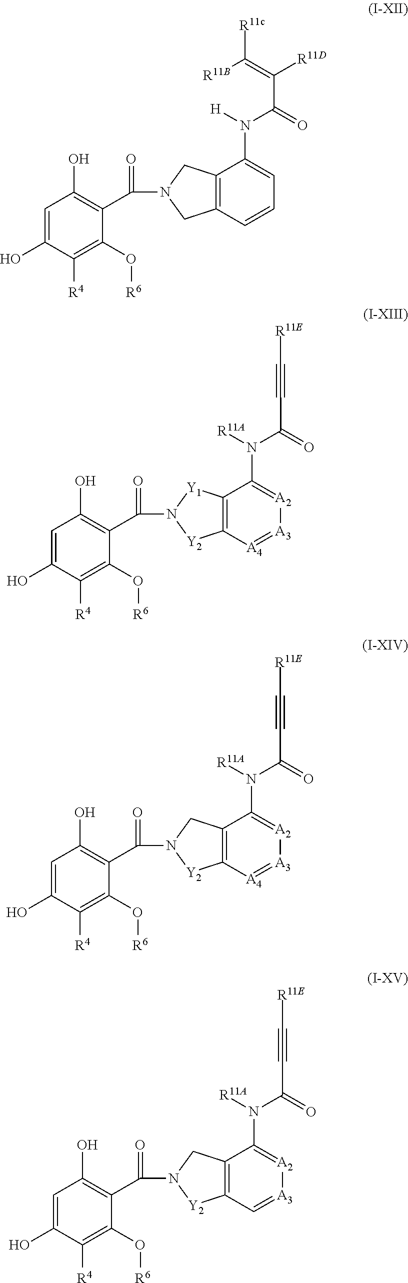 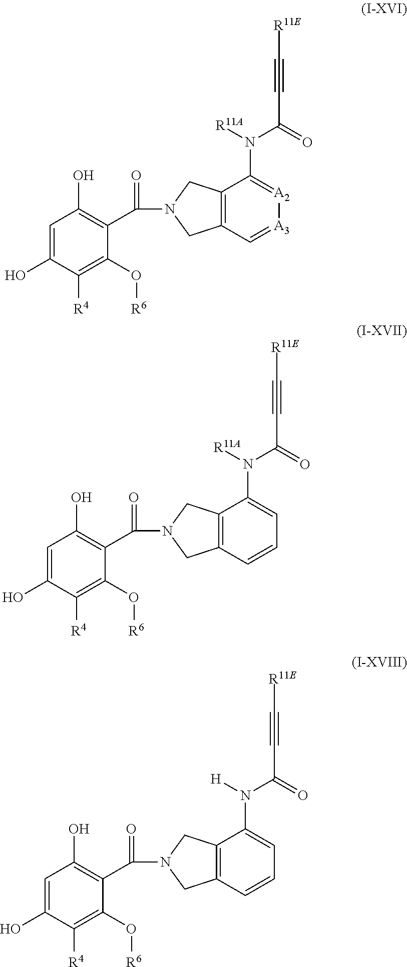
wherein R⁴, R⁶, Y₁, Y₂, A₂, A₃, A₄, R¹¹, R^{11A}, R^{11B}, R^{11C}, R^{11D} and R^{11E} are each as defined in claim 1.

4. A compound according to claim 1, wherein the compound is a compound of the formula II-I, II-II, II-III, II-IV, II-V or II-VI (which are sub-definitions of formula II), or a pharmaceutically acceptable salt, hydrate and/or solvate thereof: 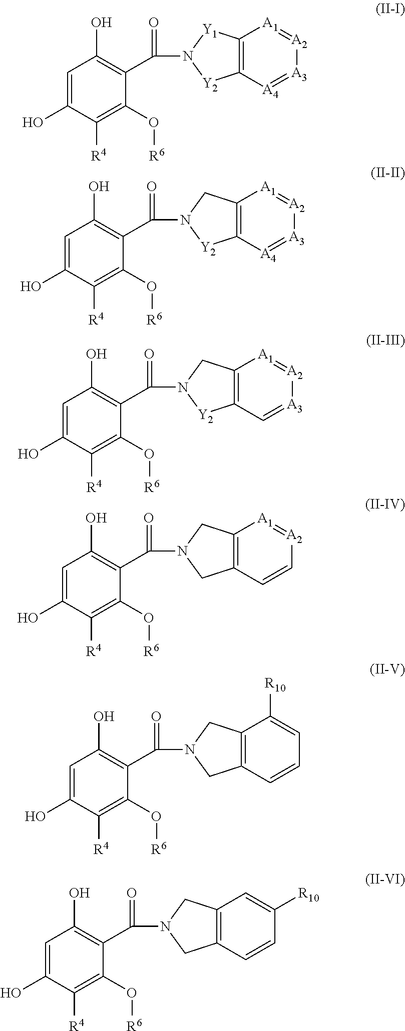
wherein R⁴, R⁶, R¹⁰, Y₁, Y₂, A₁, A₂, A₃, and A₄, are each as defined in claim 1.

5. A compound according to any one of the preceding claims, or pharmaceutically acceptable salt, hydrate or solvate thereof, wherein R⁶ is (1-6C)alkyl, (1-6C)haloalkyl, (3-6C)cycloalkyl, or a 4- to 6-membered heterocyclyl ring comprising one heteroatom selected from N, O or S,
or a group having a structure according to formula (A) shown below: 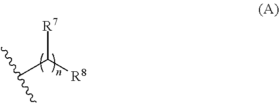
wherein
R⁷ is hydrogen or (1-3C)alkyl;
n is 1 or 2;
R³ is (3-8C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy.

6. A compound according to any one of the preceding claims, or pharmaceutically acceptable salt, hydrate or solvate thereof, wherein R⁶ is a group having a structure according to formula (A) shown below: 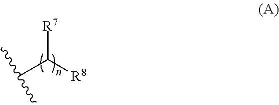
wherein
R⁷ is hydrogen or methyl;
n is 1;
R⁸ is cyclohexyl, phenyl, 6-membered heterocyclyl, or pyridyl, each of which is optionally substituted with one or more R⁹; where each R⁹ is independently selected from the group consisting of halogen, methyl, methoxy, trifluoromethyl, or trifluoromethoxy.

7. A compound according to any one of the preceding claims, or pharmaceutically acceptable salt, hydrate or solvate thereof, wherein Y₁ is —CH₂—.

8. A compound according to any one of the preceding claims, or pharmaceutically acceptable salt, hydrate or solvate thereof, wherein Y₂ is —CH₂—, —CHR_{y2a}—, —CH₂—CH₂—, —CH₂—CHR_{y2b}—, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl.

9. A compound according to any one of the preceding claims, or pharmaceutically acceptable salt, hydrate or solvate thereof, wherein Y₁ is —CH₂— and Y₂ is —CH₂—.

10. A compound according to any one of claims 1 or 4 to 9, or pharmaceutically acceptable salt, hydrate or solvate thereof, wherein A₂, A₃ or A₄ are selected from one of the following options:
(i) A₁, is selected from N, CH or CR¹⁰;
A₂ is selected from N, CH or CR¹⁰;
A₃ is selected from N or CH;
A₄ is selected from N, CH or CR¹⁴;
with the proviso that:
only one or two of A₁, A₂, A₃ or A₄ can be N; and
one of A₁, and A₂ is CR¹⁰;
(ii) A₁ is selected from N, CH or CR¹⁰;
A₂ is selected from N, CH or CR¹⁰;
A₃ is selected from N, CH or CR¹³;
A₄ is selected from N or CH;
with the proviso that:
only one or two of A₁, A₂, A₃ or A₄ can be N; and
one of A₁, and A₂ is CR¹⁰;
(iii) A₁ is selected from N, CH or CR¹⁰;
A₂ is selected from N, CH or CR¹⁰;
A₃ is selected from N or CH;
A₄ is selected from N or CH;
with the proviso that:
only one or two of A₁, A₂, A₃ or A₄ can be N; and
one of A₁, and A₂ is CR¹⁰;
(iv) A₁, is selected from CH or CR¹⁰;
A₂ is selected from CH or CR¹⁰;
A₃ is CH;
A₄ is CH;
with the proviso that:
only one of A₁ and A₂ can be CR¹⁰;
(v) A₁ is CR¹⁰;
A₂ is CH;
A₃ is CH;
A₄ is CH; or
(vi) A₁ is CH;
A₂ is CR¹⁰;
A₃ is CH;
A₄ is CH;

11. A compound according to any one of the preceding claims, or pharmaceutically acceptable salt, hydrate or solvate thereof, wherein A₂, A₃ and A₄ are all CH or A₁, A₃ and A₄ are all CH.

12. A compound according to any one of the preceding claims, or pharmaceutically acceptable salt, hydrate or solvate thereof, wherein R¹⁰ is a group of the formula (IIA) or (IIB) shown below: 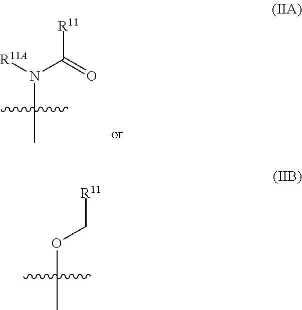
wherein [symbol] denotes the point of attachment to A₁ or A₂, and wherein R¹¹ and R^{11A} are as defined in claim 1.

13. A compound according to any one of the preceding claims, or pharmaceutically acceptable salt, hydrate or solvate thereof, wherein R¹⁰ is one of the groups shown below: 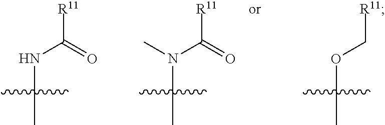
wherein [symbol] denotes the point of attachment to A₁ or A₂, and wherein R¹¹ is as defined herein in claim 1.

14. A compound according to any one of claims 1 to 12, or pharmaceutically acceptable salt, hydrate or solvate thereof, wherein R^{11A} is hydrogen or methyl.

15. A compound according to any one of the preceding claims, or pharmaceutically acceptable salt, hydrate or solvate thereof, wherein R^{11B} and R^{11C} are each independently selected from the group consisting of hydrogen, halo, (1-4C)alkyl, and —C(O)NR^{11F}R^{11G}, wherein R^{11F} and R^{11G} are each independently selected from hydrogen or (1-4C)alkyl;
and wherein any (1-4C)alkyl group present in a R^{11B} and/or R^{11C} group is optionally substituted with one or more substituents selected from halo, hydroxy, cyano, [(1-4C)alkyl]amino, di-[(1-4C)alkyl]amino, (1-4C)alkoxy, or a 4, 5 or 6-membered N-linked heterocycle which is optionally further substituted by one or more substituents selected from halo, hydroxy, cyano, (1-4C)alkyl, or (1-4C)alkoxy.

16. A compound according to any one of the preceding claims, or pharmaceutically acceptable salt, hydrate or solvate thereof, wherein one of R^{11B} and Ru^{1C} is hydrogen and the other is selected from the group consisting of hydrogen, halo, (1-2C)alkyl and —C(O)NR^{11F}R^{11G} wherein R^{11F} and R^{11G} are each independently selected from hydrogen or (1-2C)alkyl;
and wherein any (1-2C)alkyl group present in a R^{11B} and/or R^{11C} group is optionally substituted with one or more substituents selected from halo, hydroxy, cyano, [(1-2C)alkyl]amino, di-[(1-2C)alkyl]amino, (1-2C)alkoxy, or a 4, 5 or 6-membered N-linked heterocycle which is optionally further substituted by one or more substituents selected from halo, hydroxy, cyano, (1-2C)alkyl, or (1-2C)alkoxy.

17. A compound according to any one of the preceding claims, or pharmaceutically acceptable salt, hydrate or solvate thereof, wherein one of R^{11B} and R^{11C} is hydrogen and the other is selected from the group consisting of hydrogen, fluoro, (1-2C)alkyl and —C(O)NR^{11F}R^{11G}, wherein R^{11F} and R^{11G} are each independently selected from hydrogen or (1-2C)alkyl;
and wherein any (1-2C)alkyl group present in a R^{11B} and/or R^{11C} group is optionally substituted with one or more substituents selected from chloro, fluoro, [(1-2C)alkyl]amino, di-[(1-2C)alkyl]amino, (1-2C)alkoxy or a 5 or 6-membered N-linked heterocycle which is optionally further substituted by one or more substituents selected from chloro, fluoro, hydroxy, cyano, (1-2C)alkyl, or (1-2C)alkoxy

18. A compound according to any one of claims 1 to 13, or pharmaceutically acceptable salt, hydrate or solvate thereof, wherein R^{11E} is (1-4C)alkyl is optionally substituted with halo, hydroxy, cyano, [(1-4C)alkyl]amino, di-[(1-4C)alkyl]amino, (1-4C)alkoxy or a 4, 5 or 6-membered N-linked heterocycle which is optionally further substituted by one or more substituents selected from halo, hydroxy, cyano, (1-4C)alkyl or (1-4C)alkoxy.

19. A compound according to any one of claims 1 to 13, or pharmaceutically acceptable salt, hydrate or solvate thereof, wherein R^{11E} is (1-4C)alkyl.

20. A compound according to any one of claims 1 to 17, or pharmaceutically acceptable salt, hydrate or solvate thereof, wherein R^{11D} is selected from:
(i) hydrogen, halo, (1-2C)alkyl and —C(O)H, wherein any (1-2C)alkyl or —C(O)H group is optionally substituted with one or more substituents selected from halo, hydroxy, cyano, [(1-2C)alkyl]amino, di-[(1-2C)alkyl]amino, (1-2C)alkoxy, (4-6C)cycloalkoxy, phenyl or 4- to 6-membered heterocyclyl; and wherein any phenyl or 4- to 6-membered heterocyclic ring is optionally further substituted by one or more substituents selected from halo, hydroxy, cyano, (1-2C)alkyl, (1-2C)alkoxy, (4-6C)cycloalkyl, (4-6C)cycloalkoxy or (1-2C)alkyl;
(ii) R^{11D} is selected from hydrogen, halo, (1-2C)alkyl and —C(O)H, wherein any (1-2C)alkyl or —C(O)H group is optionally substituted with one or more substituents selected from halo, hydroxy, [(1-2C)alkyl]amino, di-[(1-2C)alkyl]amino or 4- to 6-membered heterocyclyl;
(iii) R^{11D} is selected from hydrogen, chloro, fluoro, methyl and ethyl, wherein any methyl or ethyl is optionally substituted with one or more substituents selected from chloro, fluoro, [(1-2C)alkyl]amino, di-[(1-2C)alkyl]amino or a 5- or 6-membered heterocyclyl; or
(iv) R^{11D} is selected from hydrogen, fluoro or methyl, wherein any methyl is optionally substituted with one or more substituents selected from —NMe₂ or morpholine.

21. A compound according to any one of the preceding claims, or pharmaceutically acceptable salt, hydrate or solvate thereof, wherein R¹² is selected from:
(i) halo, (1-2C)alkyl, (1-2C)alkoxy, wherein any (1-2C)alkyl or (1-2C)alkoxy is optionally substituted by one or more fluoro or (1-2C)alkoxy; or
(ii) fluoro, (1-2C)alkyl, (1-2C)alkoxy.

22. A compound according to any one of the preceding claims, or pharmaceutically acceptable salt, hydrate or solvate thereof, wherein R¹³ is selected from halo, (1-2C)alkyl, (1-2C)alkoxy, wherein any (1-2C)alkyl moiety is optionally substituted by one or more halo or (1-2C)alkoxy, or
R¹³ is —(CHRₛ)ₕ—Z¹³,
wherein Rₒ is hydrogen or methyl;
wherein h is 0 or 1; and
Z¹² is —OR²⁵, —NR²⁶R²⁷, or —C(O)NR²⁶R²⁷;
wherein R²⁵ is (1-4C)alkyl, a carbon-linked 4 to 8-membered heterocyclyl, or —(CHRₙ)ⱼ-[4 to 8-membered heterocyclyl], wherein Rₙ is hydrogen or methyl and i is 0 or 1;
R²⁶ and R²⁷ are each independently selected from hydrogen, (1-6C)alkyl, a carbon-linked 4 to 8-membered heterocyclyl, or —(CHRₘ)ⱼ-[4 to 8-membered heterocyclyl], wherein Rₘ is hydrogen or methyl and j is 0 or 1;
or R²⁶ and R²⁷ are linked, such that, together with the nitrogen atom to which they are attached, they form a 4-8 membered heterocyclic ring;
wherein each of R²⁵, R²⁶, R²⁷ or any ring formed when R²⁶ and R²⁷ are linked, is optionally substituted with one or more R^{a};
wherein R^{a} is as defined in claim 1.

23. A compound according to any one of the preceding claims, or pharmaceutically acceptable salt, hydrate or solvate thereof, wherein:
(i) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen;
wherein m is 0 or 1; and
Z¹² is —OR³⁰, —NR³¹R³², —C(O)NR³¹R³² or —NR³³C(O)R³⁴;
wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[4 to 8-membered heterocyclyl], wherein R; is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1
or R³¹ and R³² are linked, such that, together with the nitrogen atom to which they are attached, they form a 4-8 membered heterocyclic ring;
R³³ is hydrogen or methyl;
R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[⁴ to 8-membered heterocyclyl], wherein Rₕ is hydrogen or methyl and q is 0 or 1;
wherein R³⁰, R³¹, R³² or R³⁴, or any ring formed when R³¹ and R³² are linked, is optionally substituted with one or more R^{a};
wherein R^{a} is as defined in claim 1; or
(ii) R¹¹ is selected from cyano, halo, (1-2C)alkyl, (1-2C)alkoxy, wherein any (1-2C)alkyl or (1-2C)alkoxy is optionally substituted by one or more halo or (1-2C)alkoxy.

24. A compound according to any one of the preceding claims, or pharmaceutically acceptable salt, hydrate or solvate thereof, wherein:
(i) each R^{a} is independently selected from the group consisting of oxo, halogen, cyano, hydroxy, (1-4C)alkyl, or a group
-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —SO₂N(R^{14a})—, or —N(R^{14a})SO₂—, where R^{14a} is hydrogen or (1-2C)alkyl;
Q¹ is selected from the group consisting of hydrogen, (1-4C)alkyl, or (3-6C)cycloalkyl;
(ii) each R^{a} is independently selected from the group consisting of oxo, halogen, cyano, hydroxy, (1-2C)alkyl, or a group
-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, —NR^{14a}—, —SO₂N(R^{14a})—, or —N(R^{14a})SO₂—, where R^{14a} is hydrogen or (1-2C)alkyl;
Q¹ is selected from the group consisting of hydrogen, (1-2C)alkyl, or (3-6C)cycloalkyl;
(iii) each R^{a} is independently selected from the group consisting of oxo, halogen, cyano, hydroxy, (1-2C)alkyl, or a group
-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R^{14a})—, —N(R^{14a})—C(O)—, or —NR^{14a}—, where R^{14a} is hydrogen or (1-2C)alkyl;
Q¹ is selected from the group consisting of hydrogen or (1-2C)alkyl; or
(iv) each R^{a} is independently selected from the group consisting of oxo, halogen, cyano, hydroxy, (1-2C)alkyl, or a group
-L¹-X¹-Q¹
wherein:
L¹ is absent;
X¹ is absent or is selected from the group consisting of —O—, —C(O)—, —S(O)₀₋₂—, —C(O)—N(R^{4a})—, —N(R^{14a})—C(O)—, or —NR^{14a}—, where R^{14a} is hydrogen or (1-2C)alkyl;
Q¹ is selected from the group consisting of hydrogen or (1-2C)alkyl.

25. A compound, or pharmaceutically acceptable salts, hydrates or solvates thereof, according to any one of the preceding claims, wherein the compound is selected from any one of the following:
N-(2-(2-(Benzyloxy)-4,6-dihydroxybenzoyl)isoindolin-4-yl)acrylamide;
(E)-N-(2-(2-(Benzyloxy)-4,6-dihydroxybenzoyl)isoindolin-4-yl)-4-(dimethylamino)but-2-enamide;
N-(2-(2-(Benzyloxy)-4,6-dihydroxy-benzoyl)isoindolin-4-yl)-2-fluoroacrylamide;
N-(2-(2-(Benzyloxy)-4,6-dihydroxy-3-methylbenzoyl)isoindolin-4-yl)acrylamide;
N-(2-(2-(Benzyloxy)-4,6-dihydroxy-3-methylbenzoyl)isoindolin-4-yl)-2-fluoroacrylamide;
(E)-N-(2-(2-(Benzyloxy)-4,6-dihydroxy-3-methylbenzoyl)isoindolin-4-yl)-4-(dimethylamino)but-2-enamide;
N-(2-(2-(cyclohexylmethoxy)-4, 6-dihydroxy-3-methylbenzoyl) isoindolin-4-yl) acrylamide;
(E)-N-(2-(2-(cyclohexylmethoxy)-4,6-dihydroxy-3-methylbenzoyl)isoindolin-4-yl)-4-(dimethylamino)but-2-enamide;
(E)-N-(2-(2-(Cyclohexylmethoxy)-4,6-dihydroxy-3-methylbenzoyl)isoindolin-4-yl)-4-(pyrrolidin-1-yl)but-2-enamide;
(E)-N-(2-(2-(cyclohexylmethoxy)-4,6-dihydroxy-3-methylbenzoyl)isoindolin-4-yl)-4-morpholinobut-2-enamide hydrochloride;
(E)-N-(2-(2-(Cyclohexylmethoxy)-4,6-dihydroxy-3-methylbenzoyl)isoindolin-4-yl)-4-(4-methylpiperazin-1-yl)but-2-enamide;
N-(2-(2-(Cyclohexylmethoxy)-4,6-dihydroxy-3-methylbenzoyl)isoindolin-4-yl)but-2-ynamide;
(E)-N-(2-(2-(Cyclohexylmethoxy)-4,6-dihydroxy-3-methylbenzoyl)isoindolin-4-yl)but-2-enamide;
(E)-N-[2-[2-(Cyclopentylmethoxy)-4,6-dihydroxy-3-methyl-benzoyl]isoindolin-4-yl]-4-(dimethylamino)but-2-enamide;
(E)-N-[2-(4,6-Dihydroxy-2-methoxy-3-methyl-benzoyl)isoindolin-4-yl]-4-(dimethylamino)but-2-enamide;
N-[2-[2-(Cyclohexylmethoxy)-4,6-dihydroxy-3-methyl-benzoyl]isoindolin-5-yl]prop-2-enamide;
N-[2-[2-(Cyclohexylmethoxy)-4,6-dihydroxy-3-methyl-benzoyl]-3,4-dihydro-1H-isoquinolin-8-yl]prop-2-enamide;
N-[2-[2-(Cyclohexylmethoxy)-4,6-dihydroxy-3-methyl-benzoyl]-3,4-dihydro-1H-isoquinolin-7-yl]prop-2-enamide;
(E)-N-[2-[2-(Cyclohexylmethoxy)-4,6-dihydroxy-3-methyl-benzoyl]isoindolin-5-yl]-4-(dimethylamino)but-2-enamide;
(E)-N-[2-[2-(Cyclohexylmethoxy)-4,6-dihydroxy-3-methyl-benzoyl]-3,4-dihydro-1H-isoquinolin-8-yl]-4-(dimethylamino)but-2-enamide;
(E)-N-[2-[2-(Cyclohexylmethoxy)-4,6-dihydroxy-3-methyl-benzoyl]-3,4-dihydro-1H-isoquinolin-7-yl]-4-(dimethylamino)but-2-enamide;
(E)-4-(Dimethylamino)-N-[2-(2-ethoxy-4,6-dihydroxy-3-methyl-benzoyl)isoindolin-4-yl]but-2-enamide;
(E)-N-[2-[4,6-Dihydroxy-3-methyl-2-(2,2,2-trifluoroethoxy)benzoyl]isoindolin-4-yl]-4-(dimethylamino)but-2-enamide;
(E)-N-[2-[2-(cyclopropylmethoxy)-4,6-dihydroxy-3-methyl-benzoyl]isoindolin-4-yl]-4-(dimethylamino)but-2-enamide;
(E)-N-[2-(4,6-Dihydroxy-2-methoxy-3-methyl-benzoyl)isoindolin-4-yl]-4-(dimethylamino)-N-methyl-but-2-enamide;
(E)-N-[2-(4,6-Dihydroxy-2-methoxy-3-methyl-benzoyl)isoindolin-5-yl]-4-(dimethylamino)but-2-enamide;
(E)-N-[2-(4,6-dihydroxy-2-methoxy-3-methyl-benzoyl)isoindolin-5-yl]-4-(dimethylamino)-N-methyl-but-2-enamide;
(E)-N-[2-(4,6-dihydroxy-2-methoxy-3-methyl-benzoyl)-3,4-dihydro-1H-isoquinolin-7-yl]-4-(dimethylamino)but-2-enamide;
(E)-N-[2-(4,6-dihydroxy-2-methoxy-3-methyl-benzoyl)-3,4-dihydro-1H-isoquinolin-7-yl]-4-(dimethylamino)-N-methyl-but-2-enamide;
(E)-N-[2-[2-(Cyclohexylmethoxy)-4,6-dihydroxy-3-methyl-benzoyl]isoindolin-4-yl]-4-(dimethylamino)-N-methyl-but-2-enamide;
(E)-N-[2-[2-(Cyclopropylmethoxy)-4,6-dihydroxy-3-methyl-benzoyl]isoindolin-4-yl]-4-(dimethylamino)-N-methyl-but-2-enamide;
N-[2-(4,6-dihydroxy-2-methoxy-3-methyl-benzoyl)isoindolin-4-yl]-N-methyl-prop-2-enamide;
(E)-4-(Dimethylamino)-N-(2-(2-ethoxy-4,6-dihydroxy-3-methylbenzoyl)isoindolin-4-yl)-N-methylbut-2-enamide;
(E)-4-[2-(4,6-dihydroxy-2-methoxy-3-methyl-benzoyl)isoindolin-5-yl]oxy-N,N-dimethyl-but-2-enamide;
(E)-4-((2-(4,6-Dihydroxy-2-methoxy-3-methylbenzoyl)isoindolin-4-yl)oxy)-N, N-dimethylbut-2-enamide;
N-(2-(4,6-Dihydroxy-2-methoxy-3-methylbenzoyl) isoindolin-4-yl)-2-((dimethyl amino)methyl) acrylamide;
N-(2-(4,6-Dihydroxy-2-methoxy-3-methylbenzoyl)isoindolin-4-yl)-2-(morpholinomethyl)acrylamide.

26. A pharmaceutical composition comprising a compound as defined in any one of the preceding claims, or a pharmaceutically acceptable salt, hydrate or solvate thereof, in admixture with a pharmaceutically acceptable diluent or carrier.

27. A compound, or a pharmaceutically acceptable salt, hydrate or solvate thereof, as defined in any one of claims 1-25, or a pharmaceutical composition as defined in claim 26:
(i) for use in therapy;
(ii) for use in the treatment of cancer;
(iii) for use in the treatment of cancer, wherein the compound or pharmaceutical composition is administered in combination with another anticancer agent (e.g. a chemotherapeutic agent, an immune checkpoint inhibitor, an immune stimulator or DNA damage repair modulator);
(iv) for use in the treatment of a triplet repeat disorder.
